(19) United States
(12) Patent Application Publication
(10) Pub. No.: US 2011/0039795 A1
(43) Pub. Date: Feb. 17, 2011

(54) 2'-O,3'-N-BRIDGED MACROLIDES

(76) Inventors: Mirjana Bukvic-Krajacic, Zagreb (HR); Antun Hutinec, Zagreb (HR); Goran Kragol, Zagreb (HR); Nedjeljko Kujundzic, Zagreb (HR); Zorica Marusic-Istuk, Zagreb (HR)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(21) Appl. No.: 12/988,908

(22) PCT Filed: Apr. 20, 2009
(86) PCT No.: PCT/EP2009/054685

§ 371 Date: Oct. 21, 2010

Related U.S. Application Data
(60) Provisional application No. 61/047,216, filed on Apr. 23, 2008.

(51) Int. Cl. A61K 31/7048; C07H 17/00; A61K 31/7052; C07H 17/08; A61P 29/00; A61P 9/10; A61P 11/00; A61P 11/06

(52) U.S. Cl. 514/29; 536/7.4

(57) ABSTRACT

Novel 2′-O,3′-N-bridged macrolides useful in treatment of inflammatory diseases. More particularly, the invention relates to 2′-O,3′-N-bridged 14-membered macrolides and to 2′-O,3′-N-bridged 15-membered azalide macrolides useful in treatment of neutrophil dominated inflammatory diseases resulting from neutrophilic infiltration and/or diseases associated with altered cellular functionality of neutrophils, to intermediates for their preparation, to the methods for their preparation, to their use as therapeutic agents, and to salts thereof.

### FIELD OF THE INVENTION

The present invention relates to novel 2′-O,3′-N-bridged macrolides useful in the treatment of inflammatory diseases. More particularly, the invention relates to 2′-O,3′-N-bridged 14-membered macrolides and to 2′-O,3′-N-bridged 15-membered azalide macrolides useful in the treatment of neutrophil dominated inflammatory diseases, especially in the treatment of neutrophil dominated inflammatory diseases resulting from neutrophilic infiltration and/or diseases associated with altered cellular functionality of neutrophils, to intermediates for their preparation, to the methods for their preparation, to their use as therapeutic agents, and to salts thereof.

### BACKGROUND OF THE INVENTION

Inflammation is the final common pathway of various insults, such as infection, trauma, and allergies to the human body. It is characterized by activation of the immune system with recruitment and activation of inflammatory cells and production of pro-inflammatory mediators.

Most inflammatory diseases are characterized by enhanced accumulation of differing proportions of inflammatory cells, including monocytes/macrophages, granulocytes, plasma cells, lymphocytes and platelets. Along with tissue endothelial cells and fibroblasts, these inflammatory cells release a complex array of lipids, growth factors, cytokines and destructive enzymes that cause local tissue damage.

One form of inflammatory response is neutrophilic inflammation which is characterized by infiltration of the inflamed tissue by neutrophil polymorphonuclear leucocytes (PMN, i.e neutrophils), which are a major component of host defence. Neutrophils are activated by a great variety of stimuli and are involved in a number of clinical conditions and diseases where they play a pivotal role. Such diseases may be classified according to the major neutrophil-activating event (Table 3, page 638 of V. Witko-Sarsat et al., Laboratory Investigation (2000) 80(5), 617-653). Tissue infection by extracellular bacteria represents the prototype of this inflammatory response. On the other hand, various non-infectious diseases are characterized by extravascular recruitment of neutrophils. These non-infectious inflammatory diseases may be the result of an intermittent resurgence (e.g. flare in autoimmune diseases such as rheumatoid arthritis), or continuous generation (e.g. chronic obstructive pulmonary disease (COPD)) of inflammatory signals arising from underlying immune dysfunction. Non-infectious inflammatory diseases include chronic obstructive pulmonary disease (COPD), cystic fibrosis (CF), diffuse panbronchiolitis (DPB), bronchiolitis obliterans (BOS), bronchitis, bronchiectasis, emphysema, adult respiratory distress syndrome (ARDS, known also as acute respiratory distress syndrome), as well as glomerulonephritis, rheumatoid arthritis, gouty arthritis, ulcerative colitis, certain dermatoses such as psoriasis and vasculitis. In these conditions neutrophils are thought to play a crucial role in the development of tissue injury which, when persistent, can lead to the irreversible destruction of the normal tissue architecture with consequent organ dysfunction. Consequently, correlation between neutrophil number in sputum or bronchoalveolar lavage fluid and disease severity and decline in lung function is demonstrated in patients with chronic obstructive pulmonary disease (Di Stefano et al., Am J Respir Crit Care Med. (1998), 158(4): 1277-1285), cystic fibrosis (Sagel S D et al., J Pediatr. (2002), 141(6): 811-817), diffuse panbronchiolitis (Yanagihara K et al., Int J Antimicrob Agents. (2001), 18 Suppl 1: S83-87), bronchiolitis obliterans (Devouassoux G et al., Transpl Immunol. (2002), 10(4): 303-310), bronchitis (Thompson A B et al., Am Rev Respir Dis. (1989), 140(6): 1527-1537), bronchiectasis (Sepper R et al., Chest (1995), 107(6): 1641-1647), adult (also known as acute) respiratory distress syndrome (Weiland J E et al., Am Rev Respir Dis. (1986), 133(2): 218-225), to name a few. In addition, there is increasing evidence of neutrophil inflammation in asthmatics, particularly in patients with severe disease and patients who smoke (Chalmers G W et al., Chest (2001), 120: 1917-1922). Evidence of the importance of neutrophils in several lung diseases has prompted a search for drugs that inhibit neutrophilic infiltration into lungs and consequent inflammation (reviewed in Barnes P J, J Allergy Clin Immunol. (2007), 119(5): 1055-1062).

### BRIEF DESCRIPTION OF THE FIGURES

FIG. 1. shows correlation of inhibition of IL-6 production in vitro and inhibition of cell infiltration into BALF in vivo.

### SUMMARY OF THE INVENTION

The present invention relates to novel 2′-O,3′-N-bridged derivatives of macrolides represented by Formula (I):

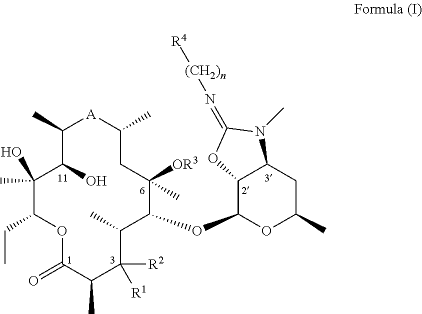

wherein,

A is a bivalent radical selected from —C(O)—, —N(R⁵)CH₂—, —CH₂N(R⁵)—, —NHC(O)—, —C(O)NH—, —CH(OH)— and —C(═NOR⁶)—;

R¹ is the α-L-cladinosyl group of formula (II);

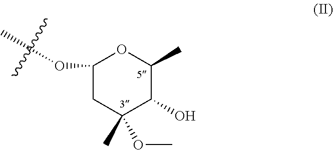

R² is hydrogen;

R³ is hydrogen or C₁₋₃alkyl;

R⁴ is
(i) C₁₋₄ alkyl optionally substituted by hydroxyl, methoxy or thiomethyl;
(ii) N,N-di(C₁-C₃-alkyl)amino;
(iii) C₆₋₁₀aryl optionally substituted by one or two groups selected from C₁₋₃alkyl, halogen, hydroxyl, C₁₋₃alkyloxy and CF₃;
(iv) a 3-6 membered monocyclic heterocyclic ring or a fused 9-10 membered bicyclic heterocyclic ring which is saturated or partially unsaturated containing one or two heteroatoms selected from oxygen, nitrogen and sulphur;
(v) a 5-6 membered monocyclic heteroaromatic ring or a fused 9-10 membered bicyclic heteroaromatic ring containing 1 to 2 heteroatoms selected from oxygen, nitrogen and sulphur;

R⁵ is C₁₋₃alkyl or hydrogen;

R⁶ is hydrogen;

n is an integer from zero to 3 provided that ‘n’ cannot be zero when R⁴ is N,N-di(C₁-C₃-alkyl)amino, or a heterocyclic ring or a heteroaromatic ring attached via an heteroatom;

or a salt thereof.

The present invention also relates to pharmaceutical compositions comprising a compound of Formula (I) or a pharmaceutically acceptable salt thereof.

Furthermore, the present invention also relates to methods of treating neutrophil dominated inflammatory diseases resulting from neutrophilic infiltration and/or diseases associated with altered cellular functionality of neutrophils comprising administration of a therapeutically effective amount of a compound of Formula (I) or a pharmaceutically acceptable salt thereof to a subject in need thereof.

According to another aspect, the invention relates to a compound of Formula (I) or a pharmaceutically acceptable salt thereof for use in human or veterinary medical therapy.

In another aspect, the invention relates to a compound of Formula (I) or a pharmaceutically acceptable salt thereof, for use in the treatment of neutrophil dominated inflammatory diseases resulting from neutrophilic infiltration and/or diseases associated with altered cellular functionality of neutrophils.

In another aspect, the invention relates to the use a compound of Formula (I) or a pharmaceutically acceptable salt thereof in the manufacture of a medicament for the treatment of neutrophil dominated inflammatory diseases resulting from neutrophilic infiltration and/or diseases associated with altered cellular functionality of neutrophils.

### DETAILED DESCRIPTION OF THE INVENTION

In one particular embodiment, the present invention is directed to the novel 2′-O,3′-N-bridged 14-membered macrolides and to 2′-O,3′-N-bridged 15-membered azalide macrolides represented by Formula (I):

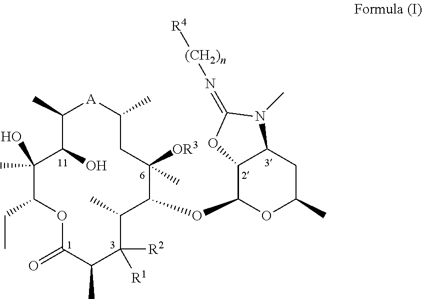

wherein,

A is a bivalent radical selected from —C(O)—, —N(R⁵)CH₂—, —CH₂N(R⁵)—, —NHC(O)—, —C(O)NH—, —CH(OH)— and —C(═NOR⁶)—;

R¹ is the α-L-cladinosyl group of formula (II);

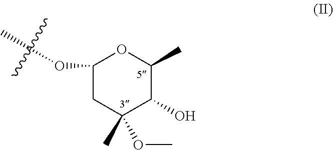

R² is hydrogen;

R³ is hydrogen or C₁₋₃alkyl;

R⁴ is
(i) C₁₋₄ alkyl optionally substituted by hydroxyl, methoxy or thiomethyl;
(ii) N,N-di(C₁-C₃alkyl)amino;
(iii) C₆₋₁₀aryl optionally substituted by one or two groups selected from C₁₋₃alkyl, halogen, hydroxyl, C₁₋₃alkyloxy and CF₃;
(iv) a 3-6 membered monocyclic heterocyclic ring or a fused 9-10 membered bicyclic heterocyclic ring which is saturated or partially unsaturated containing one or two heteroatoms selected from oxygen, nitrogen and sulphur;
(v) a 5-6 membered monocyclic heteroaromatic ring or a fused 9-10 membered bicyclic heteroaromatic ring containing 1 to 2 heteroatoms selected from oxygen, nitrogen and sulphur;

R⁵ is C₁₋₃alkyl or hydrogen;

R⁶ is hydrogen;

n is an integer from zero to 3 provided that ‘n’ cannot be zero when R⁴ is N,N-di(C₁-C₃-alkyl)amino, or heterocyclic ring or heteroaromatic ring attached via an heteroatom;

or a salt thereof.

Compounds of the present invention inhibit infiltration of neutrophils into inflamed lung tissue (as demonstrated hereinafter). Therefore these compounds have potential utility in acute and chronic treatment of inflammatory pathologies, especially of those pathologies associated with extensive neutrophil infiltration into the lung tissue, for example chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS, known also as acute respiratory distress syndrome), severe or steroid-resistant asthma (Simpson J L et al. Am J Respir Crit Care Med. (2008), 177: 148-155), and emphysema or into the respiratory tract, for example chronic rhinosinusitis (with or without nasal polyposis) (Wallwork B et al. Laryngoscope (2006), 116: 189-193). In addition, compounds of the present invention can be used for the treatment of other diseases associated with altered cellular functionality of neutrophils, for example rheumatoid arthritis (Kitsis E and, Weissmann G, Clin Orthop Relat Res. (1991), 265: 63-72), gouty arthritis, inflammatory bowel diseases (such as ulcerative colitis and Chron's disease), glomerulonephritis (Heinzelmann M et al., Am J Kidney Dis. (1999), 34(2): 384-399), damage from ischemic reperfusion (Kaminski K A et al., Int J Cardiol. (2002), 86(1): 41-59), atherosclerosis (Henriksen P A and Sallenave J M. Int J Biochem Cell (2008), 40: 1095-1100), dermatoses such as psoriasis (Terui T et al., Exp Dermatol. (2000), 9(1): 1-10) and vasculitis, systemic lupus erythematodes (SLE), systemic inflammatory response syndrome (SIRS), sepsis, ischemia-reperfusion injury, rosacea, periodontitis, gingival hyperplasia and prostatitis syndrome.

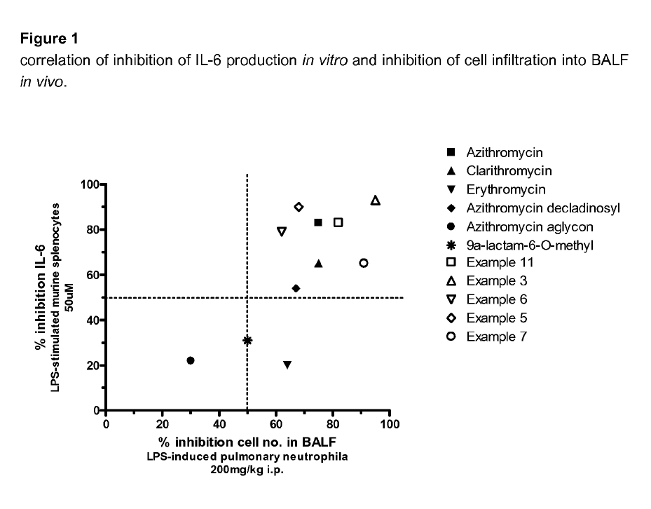

The induction of lung neutrophil infiltration in rodents by the local application of bacterial lipopolysaccharide (LPS) is widely used as a test model for neutrophilic infiltration of human lungs during pulmonary inflammatory disease. We have observed a correlation between the inhibitory activity of compounds on cell infiltration into broncho-alveolar lung fluid (BALF) of mice treated intranasally with LPS and their inhibition of interleukin-6 (IL-6) production by LPS-stimulated mouse splenocytes in vitro (FIG. 1). Therefore, inhibition of IL-6 production in LPS-stimulated murine spleen cells may be a suitable in-vitro model (biomarker) for the in-vivo activity of compounds in treating inflammatory diseases resulting from neutrophilic infiltration and/or diseases associated with altered cellular functionality of neutrophils.

A compound analyzed using the biological assays defined herein is considered to be active if it exhibits 40% or more inhibition of at least one variable, suitably 50% or more inhibition of at least one variable.

In a further embodiment the present invention also relates to 3′-N-thiocarbamoyl intermediates of formula (III) useful for the preparation of compounds of Formula (I)

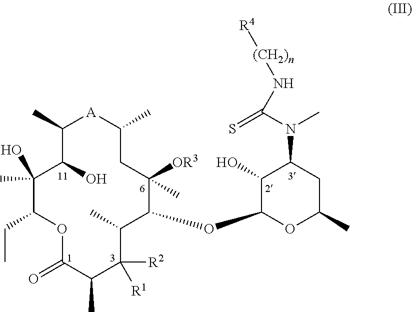

wherein,

A, R¹, R², R³, R⁴, R⁵, R⁶ and n have the meanings as defined for Formula (I) hereinabove.

In one aspect the present invention relates to a compound of Formula (I) or a salt thereof wherein the salt is a pharmaceutically acceptable salt. For a review on suitable salts see Berge at al., J. Pharm. Sci., (1977), 66: 1-19. Suitable pharmaceutically acceptable salts can include acid or base addition salts.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, salicylate, propionate, pyruvate, hexanoate, oxalate, oxaloacetate, trifluoroacetate, saccharate, glutamate, aspartate, benzoate, alkyl or aryl sulphonates (eg methanesulphonate, ethanesulphonate, benzenesulphonate or p-toluenesulphonate) and isethionate. For example, hydrochloride or acetate.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The salts of compounds of Formula (I) may form solvates (e.g. hydrates) and the invention also includes all such solvates.

In one aspect, compounds of the present invention may be in the form of pharmaceutically acceptable salts, solvates or solvates of salts. In a further aspect, a compound of Formula (I) of the present invention may be in the form of a pharmaceutically acceptable salt.

References hereinafter to “a compound according to the invention” or “compounds of the present invention” include both compound(s) of Formula (I) (whether in solvated or unsolvated form), and pharmaceutically acceptable salts (whether in solvated or unsolvated form) thereof.

With regard to stereoisomers, the compounds of Formula (I) have more than one asymmetric carbon atom. In the general Formula (I) as drawn, the solid wedge shaped bond indicates that the bond is above the plane of the paper. The broken bond indicates that the bond is below the plane of the paper.

It will be appreciated that the substituents on the macrolide may also have one or more asymmetric carbon atoms. Thus, the compounds of Formula (I) may occur as individual enantiomers or diastereomers, or mixtures thereof including racemic mixtures. All such isomeric forms are included within the present invention, including mixtures thereof.

Separation of diastereoisomers may be achieved by conventional techniques, e.g. by fractional crystallisation, chromatography or H.P.L.C. An individual stereoisomer may also be prepared from a corresponding optically pure intermediate or by resolution, such as H.P.L.C., of the corresponding mixture using a suitable chiral support or by fractional crystallisation of the diastereoisomeric salts formed by reaction of the corresponding mixture with a suitable optically active acid or base, as appropriate.

It will be appreciated that compounds of the invention may exist as geometric isomers (cis/trans or (E)/(Z)). The present invention includes the individual geometric isomers of the compounds of the invention and, where appropriate, mixtures thereof.

The compounds of Formula (I) may be in crystalline or amorphous form. Furthermore, some of the crystalline forms of the compounds of Formula (I) may exist as polymorphs, which are included in the present invention.

In one aspect of the invention A is a bivalent radical selected from —N(R⁵)CH₂—, —C(O)— and —NHC(O)—.

In one aspect of the invention A is a bivalent radical selected from —CH(OH)—, —C(O)NH— and —CH₂N(R⁵)—.

In one aspect of the invention A is a bivalent radical —N(R⁵)CH₂— wherein R⁵ is C₁₋₃alkyl, and R³ is hydrogen. In a further aspect of the invention A is a bivalent radical —N(R⁵)CH₂— wherein R⁵ is methyl, and R³ is hydrogen.

In one aspect of the invention A is a bivalent radical —N(R⁵)CH₂— wherein R⁵ is methyl, R³ is hydrogen and R⁴ is C₁₋₄alkyl. In a further aspect of the invention A is a bivalent radical —N(R⁵)CH₂— wherein R⁵ is methyl, R³ is hydrogen and R⁴ is methyl, ethyl, isopropyl or tert-butyl. In a yet further aspect of the invention A is a bivalent radical —N(R⁵)CH₂— wherein R⁵ is methyl, R³ is hydrogen and R⁴ is methyl, ethyl or isopropyl.

In one aspect of the invention A is a bivalent radical —N(R⁵)CH₂— wherein R⁵ is methyl, R³ is hydrogen and R⁴ is C₆₋₁₀aryl. In a further aspect of the invention A is a bivalent radical —N(R⁵)CH₂— wherein R⁵ is methyl, R³ is hydrogen and R⁴ is C₆₋₁₀aryl selected from phenyl and naphthyl. In a yet further aspect of the invention A is a bivalent radical —N(R⁵)CH₂— wherein R⁵ is methyl, R³ is hydrogen and R⁴ is C₆₋₁₀aryl substituted by one —OCH₃ group.

In one aspect of the invention A is a bivalent radical —N(R⁵)CH₂— wherein R⁵ is methyl, R³ is hydrogen and R⁴ is a 3-6 membered monocyclic heterocyclic ring or a fused 9-10 membered bicyclic heterocyclic ring which is saturated or partially unsaturated containing one or two heteroatoms selected from oxygen, nitrogen and sulphur. In further aspect of the invention A is a bivalent radical —N(R⁵)CH₂— wherein R⁵ is methyl, R³ is hydrogen and R⁴ is a 3-6 membered monocyclic heterocyclic ring which is saturated containing one or two heteroatoms selected from oxygen, nitrogen and sulphur. In a yet further aspect of the invention A is a bivalent radical —N(R⁵)CH₂— wherein R⁵ is methyl, R³ is hydrogen and R⁴ is 6 membered monocyclic heterocyclic ring which is saturated containing two heteroatoms selected from oxygen and nitrogen.

In one aspect of the invention A is a bivalent radical —N(R⁵)CH₂— wherein R⁵ is methyl, R³ is hydrogen and R⁴ is a 5-6 membered monocyclic heteroaromatic ring or a fused 9-10 membered bicyclic heteroaromatic ring containing 1 to 2 heteroatoms selected from oxygen, nitrogen and sulphur. In a further aspect of the invention A is a bivalent radical —N(R⁵)CH₂— wherein R⁵ is methyl, R³ is hydrogen and R⁴ is a fused 9-10 membered bicyclic heteroaromatic ring containing 1 to 2 heteroatoms selected from oxygen, nitrogen and sulphur. In a yet further aspect of the invention A is a bivalent radical —N(R⁵)CH₂— wherein R⁵ is methyl, R³ is hydrogen and R⁴ is quinolinyl.

In one aspect of the invention A is a bivalent radical —N(R⁵)CH₂— wherein R⁵ is hydrogen and R³ is hydrogen. In a further aspect of the invention A is a bivalent radical —N(R⁵)CH₂— wherein R⁵ is hydrogen, R³ is hydrogen and R⁴ is C₁₋₄alkyl. In a yet further aspect of the invention A is a bivalent radical —N(R⁵)CH₂— wherein R⁵ is hydrogen, R³ is hydrogen and R⁴ is isopropyl.

In one aspect of the invention A is a bivalent radical —CH₂N(R⁵)— wherein R⁵ is methyl, R³ is methyl and R⁴ is C₁₋₄alkyl. In a yet further aspect of the invention A is a bivalent radical —CH₂N(R⁵)— wherein R⁵ is methyl, R³ is methyl and R⁴ is isopropyl.

In one aspect of the invention A is a bivalent radical —C(O)— and R³ is hydrogen or C₁₋₃alkyl. In a further aspect of the invention A is a bivalent radical —C(O)— and R³ is hydrogen or methyl.

In one aspect of the invention A is a bivalent radical —C(O)—, R³ is methyl and R⁴ is C₁₋₄alkyl. In a further aspect of the invention A is a bivalent radical —C(O)—, R³ is methyl and R⁴ is methyl, ethyl or isopropyl. In a yet further aspect of the invention A is a bivalent radical —C(O)—, R³ is methyl and R⁴ is ethyl.

In one aspect of the invention A is a bivalent radical —C(O)—, R³ is methyl and R⁴ is C₆₋₁₀aryl. In a further aspect of the invention A is a bivalent radical —C(O)—, R³ is methyl and R⁴ is unsubstituted C₆₋₁₀aryl selected form phenyl and naphthyl.

In one aspect of the invention A is a bivalent radical —C(O)—, R³ is hydrogen and R⁴ is C₆₋₁₀aryl. In a further aspect of the invention A is a bivalent radical —C(O)—, R³ is hydrogen and R⁴ is unsubstituted C₆₋₁₀aryl. In a yet further aspect of the invention A is a bivalent radical —C(O)—, R³ is hydrogen and R⁴ is phenyl.

In one aspect of the invention A is a bivalent radical —CH(OH)— and R³ is hydrogen or C₁₋₃alkyl. In a further aspect of the invention A is a bivalent radical —CH(OH)— and R³ is hydrogen or methyl.

In one aspect of the invention A is a bivalent radical —CH(OH)— and R³ is hydrogen or methyl and R⁴ is C₁₋₄alkyl. In a further aspect of the invention A is a bivalent radical —CH(OH)— and R³ is hydrogen or methyl and R⁴ is ethyl or isopropyl.

In one aspect of the invention A is a bivalent radical —CH(OH)— and R³ is hydrogen or methyl and R⁴ is C₆₋₁₀aryl. In a further aspect of the invention A is a bivalent radical —CH(OH)— and R³ is hydrogen or methyl and R⁴ is unsubstituted C₆₋₁₀aryl. In a yet further aspect of the invention A is a bivalent radical —CH(OH)— and R³ is hydrogen or methyl and R⁴ is phenyl.

In one aspect of the invention A is a bivalent radical —NHC(O)— and R³ is hydrogen or C₁₋₃alkyl. In a further aspect of the invention A is a bivalent radical —NHC(O)— and R³ is hydrogen or methyl.

In one aspect of the invention A is a bivalent radical —NHC(O)—, R³ is methyl and R⁴ is C₁₋₄alkyl. In a further aspect of the invention A is a bivalent radical —NHC(O)—, R³ is methyl and R⁴ is methyl, ethyl, isopropyl or tert-butyl. In a yet further aspect of the invention A is a bivalent radical —NHC(O)—, R³ is methyl and R⁴ is methyl, ethyl, isopropyl. In a even further aspect of the invention A is a bivalent radical —NHC(O)—, R³ is methyl and R⁴ is isopropyl.

In one aspect of the invention A is a bivalent radical —NHC(O)—, R³ is methyl, R⁴ is N,N-di(C₁-C₃alkyl)amino and n is 3. In a further aspect of the invention A is a bivalent radical —NHC(O)—, R³ is methyl, R⁴ is N,N-diethylamino and n is 3.

In one aspect of the invention A is a bivalent radical —NHC(O)—, R³ is methyl, R⁴ is C₆₋₁₀aryl (suitably phenyl or naphthyl, specifically phenyl or 1-naphthyl). In a further aspect of the invention A is a bivalent radical —NHC(O)—, R³ is methyl, and R⁴ is unsubstituted C₆₋₁₀aryl. In a yet further aspect of the invention A is a bivalent radical —NHC(O)—, R³ is methyl and R⁴ is C₆₋₁₀aryl substituted by one or two substitutents selected from halogen (suitably fluoro) or —OCH₃ group.

In one aspect of the invention A is a bivalent radical —NHC(O)—, R³ is methyl, R⁴ is a 3-6 membered monocyclic heterocyclic ring or a fused 9-10 membered bicyclic heterocyclic ring which is saturated or partially unsaturated containing one or two heteroatoms selected from oxygen, nitrogen and sulphur. In further aspect of the invention A is a bivalent radical —NHC(O)—, R³ is methyl and R⁴ is a 3-6 membered monocyclic heterocyclic ring which is saturated containing one or two heteroatoms selected from oxygen, nitrogen and sulphur. In a yet further aspect of the invention A is a bivalent radical —NHC(O)—, R³ is methyl and R⁴ is 5-6 membered monocyclic heterocyclic ring which is saturated containing one or two heteroatoms selected from oxygen and nitrogen.

In one aspect of the invention A is a bivalent radical —NHC(O)—, R³ is methyl, R⁴ is a 5-6 membered monocyclic heteroaromatic ring or a fused 9-10 membered bicyclic heteroaromatic ring containing 1 to 2 heteroatoms. In a further aspect of the invention A is a bivalent radical —NHC(O)—, R³ is methyl, R⁴ is furyl or quinolinyl.

In one aspect of the invention A is a bivalent radical —C(O)NH—, R³ is methyl and R⁴ is C₁₋₄alkyl. In a further aspect of the invention A is a bivalent radical —C(O)NH—, R³ is methyl and R⁴ is isopropyl.

In one aspect of the invention A is a bivalent radical —C(O)NH—, R³ is methyl and R⁴ is C₆₋₁₀aryl. In a further aspect of the invention A is a bivalent radical —C(O)NH—, R³ is methyl, and R⁴ is unsubstituted C₆₋₁₀aryl. In a yet further aspect of the invention A is a bivalent radical —C(O)NH—, R³ is methyl and R⁴ is phenyl.

In one aspect of the invention R³ is hydrogen.

In one aspect of the invention R³ is methyl.

In one aspect of the invention R⁴ is C₁₋₄alkyl and n is zero. In a further aspect of the invention R⁴ is methyl, ethyl, isopropyl or tert-butyl and n is zero. In a yet further aspect of the invention R⁴ is isopropyl and n is zero.

In one aspect of the invention R⁴ is C₁₋₄alkyl substituted by methoxy and n is zero. In a further aspect of the invention R⁴ is propyl substituted by methoxy and n is zero.

In one aspect of the invention R⁴ is N,N-di(C₁-C₃alkyl)amino and n is 3. In a further aspect of the invention R⁴ is N,N-diethylamino and n is 3.

In one aspect of the invention R⁴ is C₆₋₁₀aryl. In a further aspect of the invention R⁴ is phenyl or naphthyl. In a yet further aspect of the invention R⁴ is 1-naphthyl.

In one aspect of the invention R⁴ is C₆₋₁₀aryl substituted by one or two halogens. In a further aspect of the invention R⁴ is phenyl substituted by two halogens. In a yet further aspect of the invention R⁴ is phenyl substituted by two fluorine atoms. In a even further aspect of the invention R⁴ is 2,6-difluorophenyl.

In one aspect of the invention R⁴ is fused 10 membered bicyclic heteroaromatic ring containing one nitrogen atom. In a further aspect of the invention R⁴ is quinolyl. In a yet further aspect of the invention R⁴ is 4-quinolyl.

In one aspect of the invention R⁴ is 5 membered monocyclic heteroaromatic ring containing one oxygen atom. In a further aspect of the invention R⁴ is furyl. In a yet further aspect of the invention R⁴ is 1-furyl.

In one aspect of the invention R⁵ is methyl.

In one aspect of the invention R⁵ is hydrogen.

In one aspect of the invention integer n is zero, 1 or 3.

In one aspect of the invention integer n is zero.

In one aspect of the invention integer n is 1.

In one aspect of the invention integer n is 2.

In one aspect of the invention integer n is 3.

It will be understood that the present invention covers all combinations of aspects, suitable, convenient and preferred groups described herein.

The term “C₁₋₄alkyl” as used herein, refers to saturated, straight or branched-chain hydrocarbon radicals containing between one and four carbon atoms. Examples of “C₁₋₄alkyl” radicals include: methyl, ethyl, propyl, isopropyl, butyl and tert-butyl.

The term “heterocyclic ring” refers to a 3-6 membered monocyclic ring or a fused 9-10 membered bicyclic ring which may be saturated or partially unsaturated containing 1 to 2 heteroatoms selected from oxygen, nitrogen or sulphur. Examples of such monocyclic rings include pyrrolidinyl, azetidinyl, pyrazolidinyl, oxazolidinyl, piperidinyl, piperazinyl, pyranyl, morpholinyl, thiomorpholinyl, thiazolidinyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl and the like. Examples of such bicyclic rings include indolinyl, isoindolinyl, benzodioxolyl, benzopyranyl, tetrahydroisoquinolinyl and the like.

The term “aryl” as used herein refers to a C₆₋₁₀ monocyclic or bicyclic hydrocarbon ring wherein at least one ring is aromatic. Examples of such groups include phenyl, naphthyl, tetrahydronaphthalenyl and the like.

The term “heteroaromatic ring” as used herein refers to a 5-6 membered monocyclic aromatic or a fused 9-10 membered bicyclic aromatic ring containing 1 to 2 heteroatoms selected from oxygen, nitrogen and sulphur. Examples of such monocyclic aromatic rings include thienyl, furyl, pyrrolyl, imidazolyl, oxazolyl, thiazolyl, isothiazolyl, isoxazolyl, pyranyl, pyrazolyl, pyrimidyl, pyridazinyl, pyrazinyl, pyridyl, and the like. Examples of such fused aromatic rings include quinolinyl, isoquinolinyl, quinazolinyl, quinoxalinyl, cinnolinyl, phthalazinyl, naphthyridinyl, indolyl, isoindolyl, azaindolyl, indolizinyl, indazolyl, pyrrolopyridinyl, furopyridinyl, benzofuranyl, isobenzofuranyl, benzothienyl, benzoimidazolyl, benzoxazolyl, benzoisoxazolyl, benzothiazolyl, benzoisothiazolyl and the like.

The term “halogen” refers to a fluorine, chlorine, bromine or iodine atom.

The term “inert solvent” or “solvent inert to the reaction”, as used herein, refers to a solvent that cannot react with the dissolved compounds including non-polar solvent such as hexane, toluene, diethyl ether, diisopropylether, chloroform, ethyl acetate, THF, dichloromethane; polar aprotic solvents such as acetonitrile, acetone, N,N-dimethylformamide, N,N-dimethylacetamide, dimethyl sulfoxide, pyridine, and polar protic solvents such as lower alcohol, acetic acid, formic acid and water.

The term “lower alcohol”, as used herein, refers to a C₁₋₄alcohol, such as for example, methanol, ethanol, propanol, isopropanol, butanol, t-butanol, and the like.

In one aspect, the present invention comprises a compound of Formula (I) selected from:

N′-benzyl-2′-O,3′-N-(carbonimidoyl)-3′-N-demethyl-9-deoxo-9a-methyl-9a-aza-9a-homoerythromycin A;

2′-O,3′-N-(carbonimidoyl)-3′-N-demethyl-9-deoxo-9a-methyl-N′-(1-naphthyl)-9a-aza-9a-homoerythromycin A;

2′-O,3′-N-(carbonimidoyl)-3′-N-demethyl-9-deoxo-N′-isopropyl-9a-methyl-9a-aza-9a-homoerythromycin A;

2′-O,3′-N-(carbonimidoyl)-3′-N-demethyl-9-deoxo-N′-[3-(diethylamino)propyl]-9a-methyl-9a-aza-9a-homoerythromycin A;

N′-(benzyl)-2′-O,3′-N-(carbonimidoyl)-3′-N-demethyl-6-O-methyl-9a-aza-9a-homoerythromycin A;

2′-O,3′-N-(carbonimidoyl)-3′-N-demethyl-6-O-methyl-N′-(1-naphthyl)-9a-aza-9a-homoerythromycin A;

2′-O,3′-N-(carbonimidoyl)-3′-N-demethyl-N′-isopropyl-6-O-methyl-9a-aza-9a-homoerythromycin A;

2′-O,3′-N-(carbonimidoyl)-3′-N-demethyl-N′-[3-(diethylamino)propyl]-6-O-methyl-9a-aza-9a-homoerythromycin A;

N′-benzyl-2′-O,3′-N-(carbonimidoyl)-3′-N-demethyl-6-O-methyl-erythromycin A;

2′-O,3′-N-(carbonimidoyl)-3′-N-demethyl-6-O-methyl-N′-(1-naphthyl)-erythromycin A;

2′-O,3′-N-(carbonimidoyl)-3′-N-demethyl-N′-isopropyl-6-O-methyl-erythromycin A;

2′-O,3′-N-(carbonimidoyl)-3′-N-demethyl-N′-[3-(diethylamino)propyl]-6-O-methyl-erythromycin A;

2′-O,3′-N-(carbonimidoyl)-3′-N-demethyl-N′-methyl-6-O-methyl-erythromycin A;

2′-O,3′-N-(carbonimidoyl)-3′-N-demethyl-N′-ethyl-6-O-methyl-erythromycin A;

2′-O,3′-N-(carbonimidoyl)-3′-N-demethyl-N′-ethyl-6-O-methyl-9a-aza-9a-homoerythromycin A;

2′-O,3′-N-(carbonimidoyl)-3′-N-demethyl-6-O-methyl-N′-(4-quinolyl)-9a-aza-9a-homoerythromycin A;

2′-O,3′-N-(carbonimidoyl)-3′-N-demethyl-N′-(2,6-difluorophenyl)-6-O-methyl-9a-aza-9a-homoerythromycin A;

2′-O,3′-N-(carbonimidoyl)-3′-N-demethyl-6-O-methyl-N′-(tert-butyl)-9a-aza-9a-homoerythromycin A;

or a salt thereof.

In further aspect, the present invention comprises a compound of Formula (I) selected from:

2′-O,3′-N-(carbonimidoyl)-3′-N-demethyl-N′-methyl-6-O-methyl-9a-aza-9a-homoerythromycin A;

2′-O,3′-N-(carbonimidoyl)-3′-N-demethyl-6-O-methyl-N′-[2-(4-morpholinyl)ethyl]-9a-aza-9a-homoerythromycin A;

2′-O,3′-N-(carbonimidoyl)-3′-N-demethyl-N′-[3-(methyloxy)propyl]-6-O-methyl-9a-aza-9a-homoerythromycin A;

2′-O,3′-N-(carbonimidoyl)-3′-N-demethyl-N′-[3-(methylthio)propyl]-6-O-methyl-9a-aza-9a-homoerythromycin A;

2′-O,3′-N-(carbonimidoyl)-3′-N-demethyl-6-O-methyl-N′-[4-(methyloxy)phenyl]-9a-aza-9a-homoerythromycin A;

2′-O,3′-N-(carbonimidoyl)-3′-N-demethyl-6-O-methyl-N′-(tetrahydro-2-furanylmethyl)-9a-aza-9a-homoerythromycin A;

2′-O,3′-N-(carbonimidoyl)-3′-N-demethyl-N′-(2-furanylmethyl)-6-O-methyl-9a-aza-9a-homoerythromycin A;

2′-O,3′-N-(carbonimidoyl)-3′-N-demethyl-N′-isopropyl-(9S)-9-dihydroerythromycin A;

N′-benzyl-2′-O,3′-N-(carbonimidoyl)-3′-N-demethyl-(9S)-9-dihydroerythromycin A;

2′-O,3′-N-(carbonimidoyl)-3′-N-demethyl-N′-ethyl-6-O-methyl-(9S)-9-dihydroerythromycin A;

2′-O,3′-N-(carbonimidoyl)-3′-N-demethyl-N′-isopropyl-6-O-methyl-(9S)-9-dihydroerythromycin A;

N′-benzyl-2′-O,3′-N-(carbonimidoyl)-3′-N-demethyl-6-O-methyl-(9S)-9-dihydroerythromycin A;

2′-O,3′-N-(carbonimidoyl)-3′-N-demethyl-N′-isopropyl-erythromycin A;

2′-O,3′-N-(carbonimidoyl)-3′-N-demethyl-N′-ethyl-erythromycin A;

N′-Benzyl-2′-O,3′-N-(carbonimidoyl)-3′-N-demethyl-erythromycin A;

2′-O,3′-N-(carbonimidoyl)-3′-N-demethyl-N′-isopropyl-6-O-methyl-8a-aza-8a-homoerythromycin A;

N′-benzyl-2′-O,3′-N-(carbonimidoyl)-3′-N-demethyl-6-O-methyl-8a-aza-8a-homoerythromycin A;

N′-benzyl-2′-O,3′-N-(carbonimidoyl)-3′-N-demethyl-9-deoxo-9a-aza-9a-homoerythromycin A;

2′-O,3′-N-(carbonimidoyl)-3′-N-demethyl-N′-isopropyl-9-deoxo-9a-aza-9a-homoerythromycin A;

N′-benzyl-2′-O,3′-N-(carbonimidoyl)-3′-N-demethyl-6-O-methyl-9-deoxo-8a-methyl-8a-aza-8a-homoerythromycin A;

2′-O,3′-N-(carbonimidoyl)-3′-N-demethyl-N′-isopropyl-6-O-methyl-9-deoxo-8a-methyl-8a-aza-8a-homoerythromycin A;

2′-O,3′-N-(carbonimidoyl)-3′-N-demethyl-N′-isopropyl-9-deoxo-9a-aza-9a-propyl-9a-homoerythromycin A;

2′-O,3′-N-(carbonimidoyl)-3′-N-demethyl-N′-methyl-9-deoxo-9a-methyl-9a-aza-9a-homoerythromycin A;

2′-O,3′-N-(carbonimidoyl)-3′-N-demethyl-N′-ethyl-9-deoxo-9a-methyl-9a-aza-9a-homoerythromycin A;

2′-O,3′-N-(carbonimidoyl)-3′-N-demethyl-N′-[3-(methylthio)propyl]-9a-methyl-9a-aza-9a-homoerythromycin A;

2′-O,3′-N-(carbonimidoyl)-3′-N-demethyl-N′-[4-(metoxy)phenyl]-9a-methyl-9a-aza-9a-homoerythromycin A;

2′-O,3′-N-(carbonimidoyl)-3′-N-demethyl-N′-(tert-butyl)-9a-methyl-9a-aza-9a-homoerythromycin A;

2′-O,3′-N-(carbonimidoyl)-3′-N-demethyl-9a-methyl-N′-(4-quinolinyl)-9a-aza-9a-homoerythromycin A;

2′-O,3′-N-(carbonimidoyl)-3′-N-demethyl-9a-methyl-N′-[2-(4-morpholinyl)ethyl]-9a-aza-9a-homoerythromycin A; and

N′-benzyl-2′-O,3′-N-(carbonimidoyl)-3′-N-demethyl-9-deoxo-9a-aza-9a-propyl-9a-homoerythromycin A;

or a salt thereof.

“Treating” or “treatment” of neutrophil dominated inflammatory diseases, especially those resulting from neutrophilic infiltration and/or diseases associated with altered cellular functionality of neutrophils means the alleviation of the symptoms and/or retardation of progression of the disease.

In one aspect, inflammatory diseases resulting from neutrophilic infiltration and/or diseases associated with altered cellular functionality of neutrophils include chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome.

In a further aspect, inflammatory diseases resulting from neutrophilic infiltration and/or diseases associated with altered cellular functionality of neutrophils include chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe asthma, emphysema, glomerulonephritis, rheumatoid arthritis, gouty arthritis, ulcerative colitis, damage from ischemic reperfusion, atherosclerosis, and dermatoses such as psoriasis and vasculitis.

In one aspect, the present invention provides a method of treating chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome in a subject in need of such treatment comprising administering to the subject a therapeutically effective amount of a compound of formula (I) or a pharmaceutically acceptable salt thereof.

In one aspect, the present invention provides a method of treating chronic obstructive pulmonary disease, cystic fibrosis, diffuse panbronchiolitis, bronchiolitis obliterans, bronchitis, bronchiectasis, adult respiratory distress syndrome, severe or steroid-resistant asthma, emphysema and chronic rhinosinusitis (with or without nasal polyposis) in a subject in need of such treatment comprising administering to the subject a therapeutically effective amount of a compound of formula (I) or a pharmaceutically acceptable salt thereof.

In one aspect, the present invention provides a method of treating chronic obstructive pulmonary disease, cystic fibrosis, diffuse panbronchiolitis, bronchiolitis obliterans, bronchitis, bronchiectasis, adult respiratory distress syndrome, severe asthma and emphysema.

In one aspect of the invention the present invention provides a method of treating chronic obstructive pulmonary disease.

In a further aspect, the present invention provides a method of treating bronchiolitis obliterans in a subject in need of such treatment comprising administering to the subject a therapeutically effective amount of a compound of formula (I) or a pharmaceutically acceptable salt thereof.

In a further aspect, the present invention provides a method of treating severe or steroid-resistant asthma in a subject in need of such treatment comprising administering to the subject a therapeutically effective amount of a compound of formula (I) or a pharmaceutically acceptable salt thereof.

In a further aspect, the present invention provides a method of treating cystic fibrosis in a subject in need of such treatment comprising administering to the subject a therapeutically effective amount of a compound of formula (I) or a pharmaceutically acceptable salt thereof.

In one aspect, the present invention provides a method of treating chronic rhinosinusitis (with or without nasal polyposis) in a subject in need of such treatment comprising administering to the subject a therapeutically effective amount of a compound of formula (I) or a pharmaceutically acceptable salt thereof.

In one aspect of the invention the present invention provides a method of treating psoriasis.

In a further aspect, the present invention provides a compound of Formula (I) or a pharmaceutically acceptable salt thereof for use in medical therapy.

In a further aspect, the present invention provides the use of a compound of Formula (I) or a pharmaceutically acceptable salt thereof in the manufacture of a medicament for the treatment of chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome.

In a further aspect, the present invention provides the use of a compound of Formula (I) or pharmaceutically acceptable salt thereof in the manufacture of a medicament for the treatment of chronic obstructive pulmonary disease, cystic fibrosis, diffuse panbronchiolitis, bronchiolitis obliterans, bronchitis, bronchiectasis, adult respiratory distress syndrome, severe or steroid-resistant asthma, emphysema and chronic rhinosinusitis (with or without nasal polyposis).

In a further aspect, the present invention provides the use of a compound of Formula (I) or pharmaceutically acceptable salt thereof in the manufacture of a medicament for the treatment of chronic obstructive pulmonary disease, cystic fibrosis, diffuse panbronchiolitis, bronchiolitis obliterans, bronchitis, bronchiectasis, adult respiratory distress syndrome, severe asthma and emphysema.

In a further aspect, the present invention provides the use of a compound of Formula (I) or pharmaceutically acceptable salt thereof in the manufacture of a medicament for the treatment of chronic obstructive pulmonary disease.

In a further aspect, the present invention provides the use of a compound of Formula (I) or pharmaceutically acceptable salt thereof in the manufacture of a medicament for the treatment of bronchiolitis obliterans.

In a further aspect, the present invention provides the use of a compound of Formula (I) or pharmaceutically acceptable salt thereof in the manufacture of a medicament for the treatment of severe or steroid-resistant asthma.

In a further aspect, the present invention provides the use of a compound of Formula (I) or pharmaceutically acceptable salt thereof in the manufacture of a medicament for the treatment of cystic fibrosis.

In a further aspect, the present invention provides the use of a compound of Formula (I) or pharmaceutically acceptable salt thereof in the manufacture of a medicament for the treatment of chronic rhinosinusitis (with or without nasal polyposis).

In a further aspect, the present invention provides the use of a compound of Formula (I) or pharmaceutically acceptable salt thereof in the manufacture of a medicament for the treatment of psoriasis.

In another aspect, the present invention provides a compound of Formula (I) or a pharmaceutically acceptable salt thereof, for use in treating of chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome.

In another aspect, the present invention provides a compound of Formula (I) or a pharmaceutically acceptable salt thereof, for use in the treatment of chronic obstructive pulmonary disease, cystic fibrosis, diffuse panbronchiolitis, bronchiolitis obliterans, bronchitis, bronchiectasis, adult respiratory distress syndrome, severe or steroid-resistant asthma, emphysema and chronic rhinosinusitis (with or without nasal polyposis).

In another aspect, the present invention provides a compound of Formula (I) or a pharmaceutically acceptable salt thereof, for use in the treatment of chronic obstructive pulmonary disease, cystic fibrosis, diffuse panbronchiolitis, bronchiolitis obliterans, bronchitis, bronchiectasis, adult respiratory distress syndrome, severe asthma and emphysema.

In another aspect, the present invention provides a compound of Formula (I) or a pharmaceutically acceptable salt thereof, for use in the treatment of chronic obstructive pulmonary disease.

In a further aspect, the present invention provides a compound of Formula (I) or a pharmaceutically acceptable salt thereof, for use in the treatment of bronchiolitis obliterans.

In a further aspect, the present invention provides a compound of Formula (I) or a pharmaceutically acceptable salt thereof, for use in the treatment of severe or steroid-resistant asthma.

In a further aspect, the present invention provides a compound of Formula (I) or a pharmaceutically acceptable salt thereof, for use in the treatment of cystic fibrosis.

In a further aspect, the present invention provides a compound of Formula (I) or a pharmaceutically acceptable salt thereof, for use in the treatment of chronic rhinosinusitis (with or without nasal polyposis).

In another aspect, the present invention provides a compound of Formula (I) or a pharmaceutically acceptable salt thereof for use in treating psoriasis.

The present invention is also directed to compositions comprising a compound of Formula (I) or a pharmaceutically acceptable salt thereof in an amount effective for therapeutic treatment of chronic obstructive pulmonary disease (COPD), cystic fibrosis (CF), diffuse panbronchiolitis (DPB), bronchiolitis obliterans (BOS), bronchitis, bronchiectasis, adult respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome in a subject in need of such treatment.

In another aspect, the present invention is also directed to compositions comprising a compound of Formula (I) or a pharmaceutically acceptable salt thereof in an amount effective for therapeutic treatment of chronic obstructive pulmonary disease, cystic fibrosis, diffuse panbronchiolitis, bronchiolitis obliterans, bronchitis, bronchiectasis, acute respiratory distress syndrome, severe or steroid-resistant asthma, emphysema and chronic rhinosinusitis (with or without nasal polyposis), in a subject in need of such treatment.

The present invention is further related to a pharmaceutical composition for the treatment of chronic obstructive pulmonary disease (COPD), cystic fibrosis (CF), diffuse panbronchiolitis (DPB), bronchiolitis obliterans (BOS), bronchitis, bronchiectasis, acute respiratory distress syndrome (ARDS), severe or steroid-resistant asthma, emphysema, chronic rhinosinusitis (with or without nasal polyposis), rheumatoid arthritis, gouty arthritis, inflammatory bowel disease (ulcerative colitis and Chron's disease), glomerulonephritis, damage from ischemic reperfusion, atherosclerosis, dermatoses such as psoriasis and vasculitis, systemic lupus erythematosus (SLE), systemic inflammatory response syndrome (SIRS), sepsis, ischemia-reperfusion injury, rosacea, periodontitis, gingival hyperplasia and prostatitis syndrome comprising a compound of Formula (I) or a pharmaceutically acceptable salt thereof.

The present invention is further related to a pharmaceutical composition for the treatment of chronic obstructive pulmonary disease, cystic fibrosis, diffuse panbronchiolitis, bronchiolitis obliterans, bronchitis, bronchiectasis, acute respiratory distress syndrome, severe or steroid-resistant asthma, emphysema and chronic rhinosinusitis (with or without nasal polyposis), comprising a compound of Formula (I) or a pharmaceutically acceptable salt thereof.

The benefit to a subject to be treated is either statistically significant or at least perceptible to the subject or to the physician.

“Subject” refers to an animal, in particular a mammal and more particularly to a human or a domestic animal or an animal serving as a model for a disease (e.g., mouse, monkey, etc.). In one aspect, the subject is a human.

A “therapeutically effective amount” means the amount of a compound that, when administered to a subject for treating a neutrophil dominated inflammatory disease resulting from neutrophilic infiltration and/or diseases associated with altered cellular functionality of neutrophils is sufficient to effect such treatment. The “therapeutically effective amount” will vary depending on the disease and its severity and the age, weight, physical condition and responsiveness of the subject to be treated and will be ultimately at the discretion of the attendant physician.

Pharmaceutical Compositions

While it is possible that, for use in the methods of the invention, a compound of Formula (I) or a pharmaceutically acceptable salt thereof may be administered as the bulk substance, it is preferable to present the active ingredient in a pharmaceutical formulation, for example, wherein the agent is in admixture with at least one pharmaceutically acceptable carrier selected with regard to the intended route of administration and standard pharmaceutical practice.

Accordingly, the present invention provides a pharmaceutical composition comprising a compound of Formula (I) or a pharmaceutically acceptable salt thereof in conjunction with at least one pharmaceutically acceptable carrier.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

The phrase “pharmaceutically acceptable”, as used herein, refers to salts, molecular entities and other ingredients of compositions that are generally physiologically tolerable and do not typically produce untoward reactions when administered to a mammal (e.g., human). Suitably, as used herein, the term “pharmaceutically acceptable” means approved by a regulatory agency of the Federal or a state government or listed in the U.S. Pharmacopoeia or other generally recognized pharmacopoeia for use in mammals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The present invention is further related to a pharmaceutical composition for the treatment of a neutrophil dominated inflammatory diseases resulting from neutrophilic infiltration and/or diseases associated with altered cellular functionality of neutrophils comprising a compound of Formula (I) or a pharmaceutically acceptable salt thereof.

The present invention is even further related to a pharmaceutical composition comprising a) 10 to 2000 mg of a compound of Formula (I) or a pharmaceutically acceptable salt thereof, and b) 0.1 to 2 g of one or more pharmaceutically acceptable excipients.

It will be appreciated that pharmaceutical compositions for use in accordance with the present invention may be in the form of oral, parenteral, transdermal, inhalation, sublingual, topical, implant, nasal, or enterally administered (or other mucosally administered) suspensions, capsules or tablets, which may be formulated in conventional manner using one or more pharmaceutically acceptable carriers or excipients. In one aspect, the pharmaceutical composition is formulated for oral administration.

The compounds of the invention can be administered for immediate-, delayed-, modified-, sustained-, pulsed- or controlled-release applications.

In one aspect, oral compositions are slow, delayed or positioned release (e.g., enteric especially colonic release) tablets or capsules. This release profile can be achieved for example, by use of a coating resistant to conditions within the stomach but releasing the contents in the colon or other portion of the GI tract wherein a lesion or inflammation site has been identified. Or a delayed release can be achieved by a coating that is simply slow to disintegrate. Or the two (delayed and positioned release) profiles can be combined in a single formulation by choice of one or more appropriate coatings and other excipients. Such formulations constitute a further feature of the present invention.

Suitable compositions for delayed or positioned release and/or enteric coated oral formulations include tablet formulations film coated with materials that are water resistant, pH sensitive, digested or emulsified by intestinal juices or sloughed off at a slow but regular rate when moistened. Suitable coating materials include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof. Plasticizers such as, but not limited to polyethylene glycol, dibutylphthalate, triacetin and castor oil may be used. A pigment may also be used to color the film. Suppositories are be prepared by using carriers like cocoa butter, suppository bases such as Suppocire C, and Suppocire NA50 (supplied by Gattefossé Deutschland GmbH, D-Weil am Rhein, Germany) and other Suppocire type excipients obtained by interesterification of hydrogenated palm oil and palm kernel oil (C₈-C₁₈ triglycerides), esterification of glycerol and specific fatty acids, or polyglycosylated glycerides, and whitepsol (hydrogenated plant oils derivatives with additives). Enemas are formulated by using the appropriate active compound according to the present invention and solvents or excipients for suspensions. Suspensions are produced by using micronized compounds, and appropriate vehicle containing suspension stabilizing agents, thickeners and emulsifiers like carboxymethylcellulose and salts thereof, polyacrylic acid and salts thereof, carboxyvinyl polymers and salts thereof, alginic acid and salts thereof, propylene glycol alginate, chitosan, hydroxypropylcellulose, hydroxypropylmethylcellulose, hydroxyethylcellulose, ethylcellulose, methylcellulose, polyvinyl alcohol, polyvinyl pyrrolidone, N-vinylacetamide polymer, polyvinyl methacrylate, polyethylene glycol, pluronic, gelatin, methyl vinyl ether-maleic anhydride copolymer, soluble starch, pullulan and a copolymer of methyl acrylate and 2-ethylhexyl acrylate lecithin, lecithin derivatives, propylene glycol fatty acid esters, glycerin fatty acid esters, sorbitan fatty acid esters, polyoxyethylene sorbitan fatty acid esters, polyethylene glycol fatty acid esters, polyoxyethylene hydrated caster oil, polyoxyethylene alkyl ethers, and pluronic and appropriate buffer system in pH range of 6.5 to 8. The use of preservatives, masking agents is suitable. The average diameter of micronized particles can be between 1 and 20 micrometers, or can be less than 1 micrometer. Compounds can also be incorporated in the formulation by using their water-soluble salt forms.

Alternatively, materials may be incorporated into the matrix of the tablet e.g. hydroxypropyl methylcellulose, ethyl cellulose or polymers of acrylic and metacrylic acid esters. These latter materials may also be applied to tablets by compression coating.

Pharmaceutical compositions can be prepared by mixing a therapeutically effective amount of the active substance with a pharmaceutically acceptable carrier that can have different forms, depending on the way of administration. Pharmaceutical compositions can be prepared by using conventional pharmaceutical excipients and methods of preparation. The forms for oral administration can be capsules, powders or tablets where usual solid vehicles including lactose, starch, glucose, methylcellulose, magnesium stearate, di-calcium phosphate, mannitol may be added, as well as usual liquid oral excipients including, but not limited to, ethanol, glycerol, and water. All excipients may be mixed with disintegrating agents, solvents, granulating agents, moisturizers and binders. When a solid carrier is used for preparation of oral compositions (e.g., starch, sugar, kaolin, binders disintegrating agents) preparation can be in the form of powder, capsules containing granules or coated particles, tablets, hard gelatin capsules, or granules without limitation, and the amount of the solid carrier can vary (between 1 mg to 1 g). Tablets and capsules are the preferred oral composition forms.

Pharmaceutical compositions containing compounds of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable flavourings for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetraacetic acid (EDTA), thiourea, tocopherol and butyl hydroxyanisole.

The compounds of the invention may also, for example, be formulated as suppositories e.g., containing conventional suppository bases for use in human or veterinary medicine or as pessaries e.g., containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons.

For application topically to the skin, the compound of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and flavourings.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., a hydrofluoroalkane such as 1,1,1,2-tetrafluoroethane (HFA 134AT) or 1,1,1,2,3,3,3-heptafluoropropane (HFA 227EA), or a mixture thereof. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The pharmaceutical compositions of the invention may contain from 0.01 to 99% weight per volume of the active material. For topical administration, for example, the composition will generally contain from 0.01-10%, more preferably 0.01-1% of the active compound.

A therapeutically effective amount of the compound of the present invention can be determined by methods known in the art. The therapeutically effective quantities will depend on the age and on the general physiological condition of the subject, the route of administration and the pharmaceutical formulation used. The therapeutic doses will generally be between about 10 and 2000 mg/day and suitably between about 30 and 1500 mg/day. Other ranges may be used, including, for example, 50-500 mg/day, 50-300 mg/day, 100-200 mg/day. The daily dose as employed for acute human treatment will range from 0.01 to 40 mg/kg body weight, suitably 2 to 20 mg/kg body weight, or suitably 5 to 10 mg/kg body weight, which may be administered in one to four daily doses, for example, depending on the route of administration and the condition of the subject. When the composition comprises dosage units, each unit will contain 10 mg to 2 g of active ingredient, suitably 200 mg to 1 g of active ingredient.

Administration may be once a day, twice a day, or more often, and may be decreased during a maintenance phase of treatment of the disease, e.g. once every second or third day instead of every day or twice a day. The dose and the administration frequency will depend on the clinical signs with the reduction or absence of at least one or more, preferably more than one, clinical signs of the acute phase known to the person skilled in the art. In one aspect of the present invention, administration is once daily oral dosing.

Method of Preparation:

Compounds of Formula (I) and salts thereof may be prepared by the general methods outlined hereinafter or any method known in the art, said methods constituting a further aspect of the invention. In the following description, the groups A, R¹, R², R³, R⁴, R⁵, R⁶ and n have the meaning defined for the compounds of Formula (I) unless otherwise stated.

It will be appreciated by those skilled in the art that it may be desirable to use protected derivatives of intermediates used in the preparation of the compounds of Formula (I). Protection and deprotection of functional groups may be performed by methods known in the art. Hydroxyl or amino groups may be protected with any hydroxyl or amino protecting group (for example, as described in Green and Wuts. Protective Groups in Organic Synthesis. John Wiley and Sons, New York, 1999). The protecting groups may be removed by conventional techniques. For example, acyl groups (such as alkanoyl, alkoxycarbonyl and aryloyl groups) may be removed by solvolysis (e.g., by hydrolysis under acidic or basic conditions). Arylmethoxycarbonyl groups (e.g., benzyloxycarbonyl) may be cleaved by hydrogenolysis in the presence of a catalyst such as palladium-on-carbon. 1,2 diol groups may be protected as acetal by reaction with dimethyl acetal of N,N-dimethylacetamide (DMADMA) or dimethyl acetal of N,N-dimethylformamide (DMFDMA) which may be removed by hydrogenolysis or methanolysis at reflux (Tetrahedron Lett. 12 (1971), 813-816, Collection Czech. Chem. Commun. 32 (1967), 3159).

The synthesis of the target compound is completed by removing any protecting groups, which are present in the penultimate intermediate using standard techniques, which are well-known to those skilled in the art. The final product is then purified, as necessary, using standard techniques such as silica gel chromatography, HPLC on silica gel, and the like or by recrystallization.

Compound of Formula (I), may be prepared by an intramolecular coupling reaction of 3′-N-thiourea derivative of Formula (III),

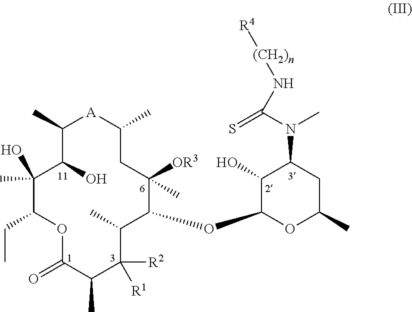

using an activating agent such as carbodiimide, such as 1-ethyl-3(3-dimethylaminopropyl)carbodiimide (EDC) or using 2-chloro-1-methylpyridinium iodide (Mukaiyama reagent, Shibanuma T et al Chem. Lett. (1977) 575-576).

The reaction may be carried out for example using 1 to 5 equivalents of 1-ethyl-3(3-dimethylaminopropyl)carbodiimide (EDC), 2-chloro-1-methylpyridinium iodide (Mukaiyama reagent), copper(II) chloride (CuCl₂) or p-toluenesulfonyl chloride (TsCl), optionally in the presence of an organic base such as triethylamine, in an inert organic solvent such as acetonitrile or halohydrocarbon (e.g. trichloromethane, dichloromethane) and at a temperature within the range from 0° C. to 80° C., suitably in the range from 40° C. to 60° C.

Compound of Formula (III), may be prepared from compound of Formula (IV)

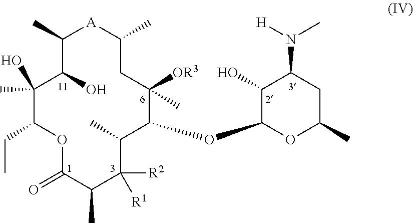

by reaction with isothiocyanate of Formula (V)

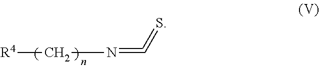

The reaction may be carried out using 2 to 4 equivalents (such as 2-3 equivalents) of isothiocyanate of Formula (V) in an inert organic solvent such as acetonitrile, dichloromethane or toluene, optionally in the presence of an organic base (such as triethylamine), at a temperature within the range from 0° C. to 80° C., suitably in the range from 40° C. to 60° C.

In yet another embodiment compound of Formula (I), may be prepared from compound of Formula (IV) using isothiocyanate of Formula (V) and 1-ethyl-3(3-dimethylaminopropyl)carbodiimide (EDC) or 2-chloro-1-methylpyridinium iodide (Mukaiyama reagent), without isolation of 3′-N-thiourea derivative of Formula (III) in one pot reaction.

Compounds of Formula (IV) are known compounds or they may be prepared by conventional techniques for mono-demethylation of the 3′-NMe₂ group, for example by reaction of compound of Formula (VI) with iodine under UV radiation (preferably with 500 W halogen lamp), in the presence of sodium acetate trihydrate (U.S. Pat. No. 3,725,385 and WO2004/013153), or by reaction of compound of Formula (VI) with iodine in the presence of an amine (suitably 2-amino-2(hydroxylmethyl)-1,3-propanediol, known as Trizma® base) (as described in WO2007/067281 and Tetrahedron Lett. (2008), 49: 598-600), or by reaction of compound of Formula (VI) with N-iodosuccinimide in acetonitrile at room temperature (J. Org. Chem. (2000), 65: 3875-3876) or with benzylchloroformate, followed by elimination of benzyloxycarbonyl groups at position 2′ and 3′ as described in U.S. Pat. No. 5,250,518.

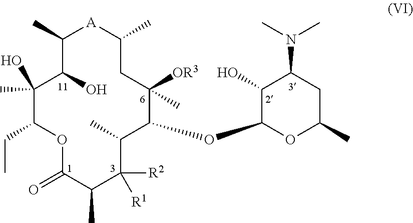

Compounds of Formula (VI) wherein A represents —C(O)— and R³ is methyl, may be prepared according to J. Antibiotics (1984), 37: 187-189.

Compounds of Formula (VI) wherein A represents —C(═NOH)— and R³ is hydrogen or methyl, may be prepared according to U.S. Pat. No. 3,478,014 or J. Antibiotics (1991), 44: 313-330.

Compounds of Formula (VI) wherein A represents —CH(OH)— may be prepared from compounds of formula (VI) where A is —C(O)— using reducing agents, for instance hydrides (sodium borohydride, lithium borohydride, sodium cyano borohydride or lithium aluminum hydride) according to J. Antibiotics (1990) 1334-1336.

Compounds of Formula (VI) wherein A represents —NHC(O)— or —C(O)NH— and R³ is methyl are known compounds or they may be prepared from corresponding 6-O-methyl compounds of Formula VI wherein A is —C(═NOH)— by Beckmann rearrangement according to the procedure described in WO99/51616.

Compounds of formula (VI) wherein A is —C(O)NH— and R³ is hydrogen are known compounds or they may be prepared from the corresponding erythromycin A (9Z)-oxime by Beckman rearrangement according to the procedure described in Bioorg. Med. Chem. Lett. (1993), 3: 1287-1292.

Compounds of Formula (VI) wherein A represents —N(R⁵)CH₂— or —NCH₂(R⁵)— and R⁵ is hydrogen may be obtained by reduction of the corresponding 9a- or 8a-imino ether and than followed by reductive N-alkylation give analogues with R⁵ is C₁₋₃alkyl according to the procedure described in J. Chem. Soc. Perkin Trans (1986) 1881-1890, J. Chem Res. S (1988) 152-153; (M) (1988) 1239-1261 and EP0508725.

Compounds of Formula V are commercially available or may be readily prepared by methods well known in the art.

Pharmaceutically acceptable acid addition salts, which also represent an object of the present invention, may be obtained by reaction of a compound of Formula (I) with an at least equimolar amount of the corresponding inorganic or organic acid such as hydrochloric acid, hydroiodic acid, sulfuric acid, phosphoric acid, acetic acid, trifluoroacetic acid, propionic acid, benzoic acid, benzenesulfonic acid, methane sulfonic acid, laurylsulfonic acid, stearic acid, palmitic acid, succinic acid, ethylsuccinic acid, lactobionic acid, oxalic acid, salicylic acid and similar acid, in a solvent inert to the reaction. Addition salts are isolated by evaporating the solvent or, alternatively, by filtration after a spontaneous precipitation or a precipitation by the addition of a non-polar cosolvent.

Compounds of Formula (I) exhibit 40% or more inhibition of interleukin-6 (IL-6) production in LPS-stimulated splenocytes treated by the compound at 50 μM or/and 25 μM concentration. Suitably compounds of Formula (I) exhibit 50% or more inhibition of interleukin-6 (IL-6) production in LPS-stimulated splenocytes treated by the compound at 50 μM or/and 25 μM concentration. Some compounds of Formula (I) exhibit more than 50% inhibition of interleukin-6 (IL-6) production in LPS-stimulated splenocytes treated by the compound at 50 μM concentration.

Biological Assays

The potential for a compound of the present invention to have an advantageous profile for providing therapeutic benefit in the treatment of neutrophil dominated inflammatory diseases resulting from neutrophilic infiltration and/or diseases associated with altered cellular functionality of neutrophils may be demonstrated, for example, using the following assays.

The following abbreviations are used in the text: DMSO for dimethyl sulfoxide, DMEM for Dulbecco's modified Eagle medium, LPS for bacterial lipopolysaccharide, PBS for phosphate buffered saline and BALF for bronchoalveolar lavage fluid.

### In Vitro Screening Protocol

Compound Preparation

Test and reference substances used in an in vitro assay are dissolved in dimethyl sulfoxide (DMSO) (Sigma Chemical Co., USA) at a concentration of 50 mM and are further diluted to final concentrations of 50 μM, 25 μM, 12.5 μM, 6.3 μM and 3.1 μM in Dulbecco's modified Eagle medium (DMEM) (Gibco, USA) supplemented with 1% heat inactivated fetal bovine serum (FBS) (BioWest, Ringmer, United Kingdom).

Inhibition of IL-6 Production in LPS-Stimulated Murine Splenocytes in Vitro

After cervical dislocation, mouse spleens were removed using sterile dissection tools. Spleens were transferred to a pre-wetted cell strainer in a 50 mL sterile conical tube and cell suspension was made by gentle puddle. Cells were centrifuged (20 min, 300×g) and resuspended in 2 mL of sterile phosphate buffered saline (PBS) (Sigma Chemical Co., USA). Red blood cells were lysed by addition of 3 mL of sterile water and occasionally gentle shaking for 1 minute. Afterwards, the tube was filled to 40 mL with DMEM medium and centrifuged (20 min, 300×g). Cells were resuspended in DMEM supplemented with 1% FBS and seeded in a 24-well plate, 1×10⁶ cells per mL medium. Cells were pre-incubated with the test compounds for 3 h at 37° C., in an atmosphere of 5% CO₂ and 90% humidity. Afterwards, cells were stimulated with 1 μg/mL lipopolysaccharide (LPS, E. coli 0111:B4, Sigma Chemical Co., USA) and incubated overnight. Concentration of IL-6 was determined in cell supernatants by sandwich ELISA using capture and detection antibodies (R&D Systems, USA) according to the manufacturer's recommendations.

Inhibition (as percentage) was calculated using the following formula:

% inhibition=[1−(concentration of IL-6 in sample−concentration of IL-6 in negative control)/(concentration of IL-6 in positive control−concentration of IL-6 in negative control)]×100.

The positive control refers to LPS-stimulated samples that were not preincubated with the compounds.

The negative control refers to unstimulated and untreated samples.

### In Vivo Screening Protocols

Lung Neutrophilia Induced by Bacterial Lipopolysaccharide (LPS) in Male BALB/cJ Mice

For intraperitoneal administration (i.p.) compounds are dissolved in a final concentration of 10 mg/mL. The required amount of compound is first dissolved in dimethylsulfoxide (DMSO, Sigma) and then diluted with 0.5% (w/v) methyl-cellulose so that the final DMSO concentration is 5% (v/v). The obtained solution is applied in a dose volume of 0.2 mL per 10 g of animal. Therefore, the compound dose is 200 mg/kg.

Male BALB/cJ mice (Charles River, France), with an average weight of ˜30 g are randomly grouped (n=8 in testing group, 10 in positive control and 8 in negative control). Mice receive intraperitoneally (i.p.) a single dose of 200 mg/kg of test compound. Two hours after administration, 2 μg of LPS (from Escherichia coli serotype 0111:B4, Sigma), dissolved in sterile PBS in a volume of 60 μL, is intranasally administered to all experimental groups except the negative control group, which receive the same volume of vehicle (PBS). Animals are sacrificed approximately 24 hours after application of LPS in order to obtain bronchoalveolar lavage fluid (BALF), which is used to determine absolute number of cells and the percentage of neutrophils. Results are expressed as percentage decrease in total cell number and number of neutrophils in BALF of treated animals compared to positive control (LPS challenged, but untreated animals).

Phorbol 12-Myristate 13-Acetate Induced Ear Edema in CD1 Mice

Male CD1 mice (Charles River, France) weighing 30-40 g are randomly grouped (n=8 in test group of which the untreated ear serves as negative control; 8 in positive control group which also serves as their own negative control group). Test compounds, as well as vehicle (Trans-phase Delivery system, containing benzyl alcohol 10%, acetone 40% and isopropanol 50%) (all from Kemika, Croatia), are administered topically to the internal surface of the left ear 30 minutes prior to administration of phorbol 12-myristate 13-acetate (PMA) (Sigma, USA). Test compounds are administered at a dose 500 μg in 15 μL per ear. 30 minutes later, 0.01% PMA solution in acetone is applied topically to the inner surface of the left ear of each animal in a volume of 12 μL per ear. During the treatment and challenge, animals are anesthetized with anesthesia by inhalation. 6 h following challenge, animals are euthanized by intraperitoneal thiopental injection (PLIVA, Croatia). For assessing the auricular edema, 8 mm discs are cut out of left and right auricular pinna and weighed. The degree of edema is calculated by subtracting the weight of 8 mm disc of the untreated ear from that of the treated contralateral ear. The inhibition of edema in the treated animals is expressed as percentage compared to control mice.

### Examples

The following abbreviations are used in the text: DMSO for dimethyl sulfoxide, EtOAc for ethyl acetate, MeOH for methanol, DCM for dichloromethane, TEA for triethylamine, DEA for diethylamine, EDC for 1-ethyl-3(3-dimethylaminopropyl)carbodiimide hydrochloride, DIPEA for N,N-diisopropylethylamine and RT for room temperature.

The compounds and processes of the present invention will be better understood in connection with the following examples, which are intended as an illustration only and not limiting the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where reactions are described as having been carried out in a similar manner to earlier, more completely described reactions, the general reaction conditions used were essentially the same. Work up conditions used were of the types standard in the art, but may have been adapted from one reaction to another. In the procedures that follow, reference to the product of a Description or Example by number is typically provided. This is provided merely for assistance to the skilled chemist to identify the starting material used. The starting material may not necessarily have been prepared from the batch referred to.

9-deoxo-9a-methyl-9a-aza-9a-homoerythromycin A, may be prepared by the procedure as described in J. Chem. Res. (S) 1988, page 152.

Intermediates:

Intermediate 1

### 3′-N-Demethyl-6-O-methyl-9a-aza-9a-homoerythromycin A

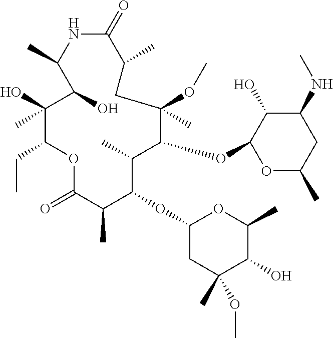

To a stirred solution of 6-O-methyl-9a-aza-9a-homoerythromycin A (10 g, 13.2 mmol), and sodium acetate trihydrate (3.7 g, 27.2 mmol) in MeOH (250 mL), solid iodine (3.9 g, 15.4 mmol) was added. The reaction mixture was irradiated with 500 W halogen lamp for 5 hours, then stirred at room temperature overnight. The solvent was evaporated, solid residue dissolved in EtOAc (250 mL), filtered, and the filtrate washed with saturated aqueous Na₂SO₃ (3×100 mL) and NaCl (1×100 mL), dried and evaporated to dryines to give crude product (1.4 g). To the combined Na₂SO₃ layer (300 mL) EtOAc (100 mL) was added (pH 5.3), pH adjusted to 9 extracted with EtOAc (2×100 mL), dried over Na₂SO₄ and evaporated to afford additional amount of the crude product (8.3 g). The combined crude products were crystalizated from EtOAc/n-hexane to afford title product (7.3 g), MS (ES+) m/z: 749.3 [M+H]⁺.

Intermediate 2

### 3′-N-Demethyl-6-O-methyl-erythromycin A

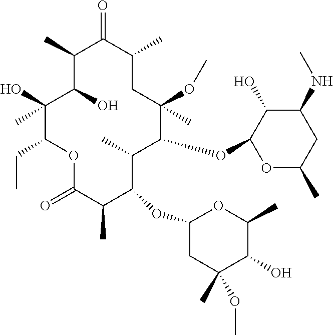

To a stirred solution of 6-O-methyl-erythromycin A (10 g, 13.4 mmol), and sodium acetate trihydrate (5.8 g, 42.6 mmol) in MeOH (250 mL), solid iodine (3.8 g, 15.0 mmol) was added. The reaction mixture was irradiated with 500 W halogen lamp for 4 hours, cooled to room temperature and the solvent evaporated. The residue was precipitated from EtOAc (500 mL) and water (400 mL), redissolved in CH₂Cl₂/MeOH/NH₄OH (90:9:1.5), impurities removed by filtration and filtrate evaporated giving crude product (12.4 g). Additional amount of crude product was obtained by evaporation of ethyl acetate layer (4.5 g). The combined crude products were purified using column chromatography (CH₂Cl₂/MeOH/NH₄OH (90:9:1.5)) to afford title product (9.35 g), MS (ES+) m/z: 734.3 [M+H]⁺.

Intermediate 3

### 3′-N-Demethyl-9-deoxo-9a-methyl-9a-aza-9a-homoerythromycin A

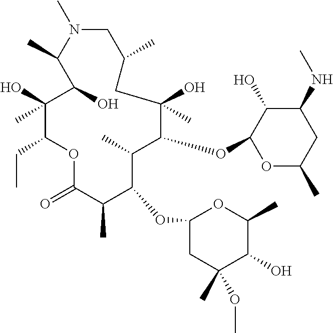

A solution of 9-deoxo-9a-methyl-9a-aza-9a-homoerythromycin A (10 g, 13.3 mmol) and N-iodosuccinimide (7.5 g, 33.3 mmol) in acetonitrile (250 mL) was stirred at room temperature for 1.5 hours. Acetonitrile was evaporated and solid residue dissolved in CH₂Cl₂ (300 mL), washed with saturated aqueous Na₂SO₃ (5×70 mL) and saturated aqueous NaHCO₃ (5×70 mL), dried over Na₂SO₄ and evaporated to afford 10.78 g of yellowish solid. The solid residue was dissolved in the mixture of water (50 mL) and CH₂Cl₂ (200 mL), pH was adjusted to 4.1, and layers separated. Extraction at pH 4.1 was repeated two times with water (2×50 mL). To the combined aqueous layers CH₂Cl₂ (200 mL) was added, pH adjusted to 9.3 and extracted with CH₂Cl₂ (100 mL). The combined organic extracts at pH 9.3 were dried over Na₂SO₄, and evaporated to afford title product (8.8 g) as a white solid, MS (ES+) m/z: 735.2 [M+H]⁺.

Intermediate 4

### 3′-N-(N′-Benzylthiocarbamoyl)-3′-N-demethyl-6-O-methyl-9a-aza-9a-homoerythromycin A

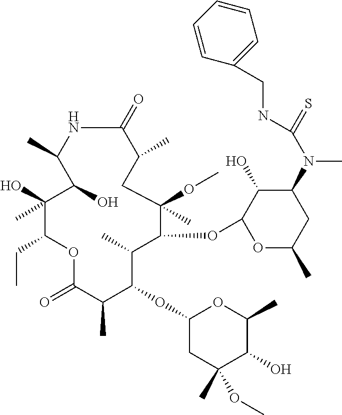

To a solution of Intermediate 1 (0.33 g, 0.44 mmol) in acetonitrile (10 mL), TEA (61 μL, 0.44 mmol) and benzyl isothiocyanate (116 μL, 0.88 mmol) were added. The reaction mixture was stirred at room temperature for 24 hours. After evaporation of the solvent crude product was purified on the Flashmaster II-solid phase extraction techniques (SPE 5 g) using gradient solvent system (98-95% CH₂Cl₂/(MeOH/NH₄OH=9/1.5) and 10 mL/min flow rate) to afford title product (260 mg), MS (ES+) m/z: 898.4 [M+H]⁺.

Intermediate 5

### 3′-N-[N′-(1-Naphthylthiocarbamoyl)]-3′-N-demethyl-9-deoxo-9a-methyl-9a-aza-9a-homoerythromycin A

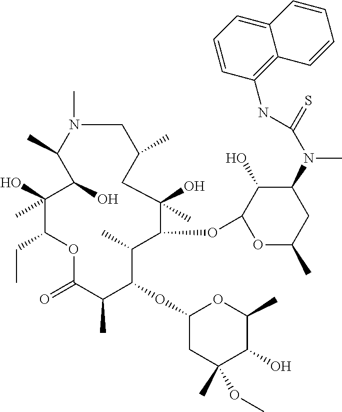

A mixture of (0.5 g, 0.68 mmol) Intermediate 3 and 1-naphthyl-isothiocyanate (0.126 g, 0.68 mmol) in dry CH₂Cl₂ (5 mL) was stirred for 30 minutes at room temperature to complete the reaction. Solvent was removed under reduced pressure. The isolation of the pure compound was performed by chromatography on a silica gel column in a solvent system CH₂Cl₂/MeOH/NH₄OH=90/9/1.5 to afford title product (0.4 g), MS (ES+) m/z: 920.7 [M+H]⁺.

Intermediate 6

### 3′-N-Demethyl-(9S)-9-dihydro-erythromycin A

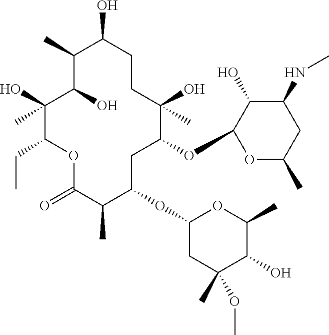

To a stirred solution of (9S)-9-dihydroerythromycin A (5 g, 6.9 mmol; may be obtained from erythromycin A according to procedure of J. Antibiotics (1990) 1334-1336) and sodium acetate trihydrate (1.85 g, 22.5 mmol) in MeOH (125 mL), solid iodine (1.95 g, 7.7 mmol) was added. The reaction mixture was irradiated with 500 W halogen lamp for 6 hours. The solvent was evaporated, solid residue dissolved in EtOAc (100 mL), and filtered. The filtrate was washed with saturated aqueous Na₂SO₃ (2×100 mL). To combined aqueous solutions fresh EtOAc (100 mL) was added, pH of the resulting mixture adjusted to 9, and layers separated. Combined organic layers were dried over anhydrous Na₂SO₄ and evaporated to afford crude product (4 g) which was purified by flash column chromatography (using MeOH/CH₂Cl₂/NH₄OH (9:90:0.5)) to afford title product (2.4 g), MS (ES+) m/z: 722.5 [M+H]⁺.

Intermediate 7

### 3′-N-Demethyl-6-O-methyl-(9S)-9-dihydroerythromycin A

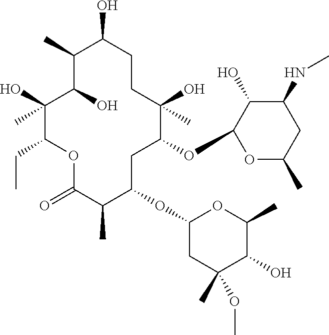

Into solution of Intermediate 2 (2.0 g, 2.73 mmol) in ethanol (50 mL), NaBH₄ (1.0 g, 27.3 mmol) was added and the reaction mixture stirred at RT for 24 hours. Solvent was evaporated, residue dissolved in CH₂Cl₂ (50 mL), water (50 mL) was added, and pH of the resulting mixture was adjusted to 2.5 (1N HCl) and then reised to 9.5 (aqueous NH₄OH). Layers were separated and aqueous one additionally extracted with CH₂Cl₂ (4×30 mL). Combined organic layers at pH 9.5 were evaporated and the residue purified by Biotage SP1 system (10 g cartridge, using CH₂Cl₂-MeOH/CH₂Cl₂/NH₄OH (7.5:90:0.75)) to afford title product (520 mg), MS (ES+) m/z: 736.14 [M+H]⁺.

¹³C NMR (125 MHz, CDCl₃) [δ/ppm] 174.9, 101.9, 96.3, 81.9, 80.1, 78.6, 78.3, 77.7, 77.1, 75.0, 74.7, 72.6, 70.8, 68.3, 65.8, 60.1, 50.7, 49.3, 45.2, 38.3, 37.2, 34.9, 34.5, 33.1, 32.4, 21.4, 21.2, 20.3, 18.6, 16.7, 16.4, 10.5, 9.7.

Intermediate 8

### 3′-N-Demethyl-erythromycin A

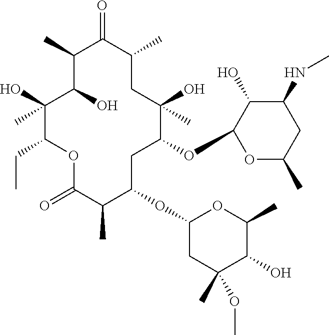

To a stirred solution of erythromycin A (10 g, 13.6 mmol) and sodium acetate trihydrate (5.9 g, 71.9 mmol) in MeOH (50 mL), solid iodine (3.8 g, 15.0 mmol) was added. The reaction mixture was irradiated with 500 W halogen lamp for 2 hours. The solvent was evaporated, solid residue dissolved in EtOAc (250 mL), and filtered. The filtrate was washed with saturated aqueous Na₂SO₃ (3×100 mL) and evaporated to afford crude product which was then purified by flash column chromatography using solvent system MeOH/CH₂Cl₂/NH₄OH (9:90:0.5) to afford title product (10.1 g), MS (ES+) m/z: 720.71 [M+H]⁺.

Intermediate 9

### 3′-N-Demethyl-6-O-methyl-8a-aza-8a-homoerythromycin A

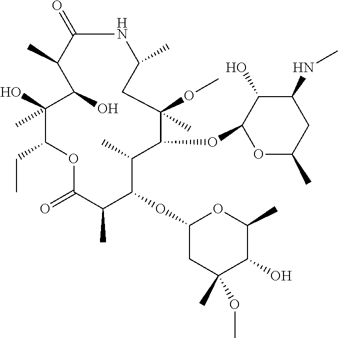

To a stirred solution of 6-O-methyl-8a-aza-8a-homoerythromycin A (0.9 g, 1.2 mmol; may be obtained according to WO99/51616, example 3), and sodium acetate trihydrate (0.33 g, 4.0 mmol) in MeOH (25 mL), solid iodine (0.35 g, 1.3 mmol) was added. The reaction mixture was irradiated with 500 W halogen lamp for 1.5 hour. The solvent was evaporated, solid residue dissolved in EtOAc (50 mL), and filtered. The filtrate was washed with saturated aqueous Na₂SO₃ (2×50 mL) and NaCl (50 mL), dried over K₂CO₃, and evaporated to afford crude product which was then precipitated from mixture EtOAc/n-hexane. The precipitate was further dissolved in CH₂Cl₂ (50 mL), water (50 mL) was added, and pH adjusted to 4.0 (1N HCl). Layers were separated and to aqueous layer fresh CH₂Cl₂ (50 mL) was added and pH adjusted to 7.3 (aq. NH₄OH). Layers were separated and aqueous layer further extracted with CH₂Cl₂ (2×50 mL). Combined organic extracts at pH 7.3 were dried over anhydrous Na₂SO₄ and evaporated. The obtained residue was precipitated from mixture EtOAc/n-hexane and then purified using column chromatography (CH₂Cl₂/MeOH/NH₄OH (90:9:1.5)) to afford title product (0.24 g), MS (ES+) m/z: 749.57 [M+H]⁺.

¹³C NMR (125 MHz, CDCl₃) [δ/ppm] 174.2, 177.3, 177.2, 102.3, 95.1, 80.0, 78.8, 77.9, 77.0, 74.7, 74.2, 72.8, 70.3, 68.5, 65.4, 60.3, 51.8, 49.3, 45.6, 42.8, 42.4, 42.2, 41.0, 37.2, 34.6, 33.1, 23.8, 21.5, 21.1, 20.9, 18.3, 16.1, 15.1, 11.0, 9.6.

Intermediate 10

### 3′-N-Demethyl-9-deoxo-9a-aza-9a-homoerythromycin A

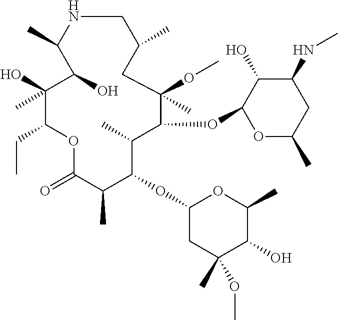

9-Deoxo-9a-aza-9a-homoerythromycin A (400 g, 0.54 mol) and Trizma® base (326 g, 2.69 mol; also known as 2-amino-2(hydroxylmethyl)-1,3-propanediol) were stirred in acetonitrile (6 L) under nitrogen. Then, iodine (612 g, 2.42 mol) was added in portions keeping the reaction mixture temperature not higher than 25° C. The reaction mixture was stirred at room temperature for 2 hours, N-iodosuccinimide (67.2 g, 0.30 mol) was added and stirring continued overnight. Then acetonitrile was removed in vacuo, brown solid residue was dissolved in ethyl acetate (3 L) and stirred with 1M aqueous potassium carbonate solution (3 L) and 12.5% sodium sulphite solution (3 L). The aqueous phase was separated and the pH adjusted from 9 to 10 by the addition of 1 M aqueous potassium carbonate (3 L). The obtained mixture was stirred for 1 hour resulting in formation of white precipitate, which was collected by filtration, washed with water (2 L) and dried overnight at 50° C. under vacuum. This material was slurried in dichloromethane (700 mL) for 1 hour, solid was collected by filtration, washed with dichloromethane (300 mL) and dryed at 50° C. under vacuo overnight to afford title product (150 g) as a white solid, MS (ES+) m/z: 721.5 [M+H]⁺.

Intermediate 11

### 3′-N-(N′-Benzylthiocarbamoyl)-3′-N-demethyl-9-deoxo-9a-aza-9a-homoerythromycin A

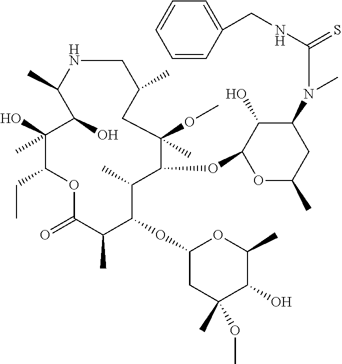

A solution of Intermediate 10 (3 g, 4.16 mmol) in acetonitrile (200 mL), was heated to 60° C., benzyl-isothiocyanate (552 μL, 4.16 mmol) was added and the reaction mixture stirred for 2 hours. Then solvent was evaporated and the residue purified by Biotage SP1 system (50 g cartridge, using CH₂Cl₂-MeOH/CH₂Cl₂/NH₄OH (7.5:90:0.75)) to afford as a second fraction crude product, which was further purified by precipitation from acetonitrile to afford title product (2.6 g).

HRMS (ES+) theoret. [M+H]⁺ C₄₄H₇₆N₃O₁₂S 870.5150, determined 870.5146 [M+H]⁺.

¹³C NMR (125 MHz, CDCl₃) [δ/ppm] 184.2, 178.4, 137.9, 128.7, 127.8, 127.5, 102.9, 95.0, 84.1, 78.0, 77.9, 77.9, 73.8, 73.5, 72.9, 72.8, 72.7, 68.0, 65.6, 60.4, 57.2, 56.6, 50.2, 49.4, 45.1, 42.0, 41.6, 36.3, 34.8, 30.9, 29.7, 27.2, 21.8, 21.6, 20.9, 20.9, 18.2, 16.0, 15.0, 14.1, 11.1, 9.6.

Intermediate 12

### 3′-N-(N′-Isopropylhiocarbamoyl)-3′-N-demethyl-9-deoxo-9a-aza-9a-homoerythromycin A

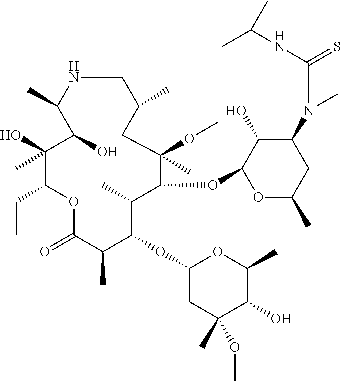

A solution of Intermediate 10 (3 g, 4.16 mmol) in acetonitrile (150 mL) was heated to 60° C., isopropyl isothiocyanate (666 μL, 4.16 mmol) was added and the reaction mixture stirred for 2 hours. Then solvent was evaporated and white foamy residue purified by Biotage SP1 system (50 g cartridge, using CH₂Cl₂-MeOH/CH₂Cl₂/NH₄OH (7.5:90:0.75)) and precipitated from acetonitrile to afford title product (2 g).

HRMS (ES+) theoret. [M+H]⁺ C₄₀H₇₆N₃O₁₂S 822.5150, determined 822.5181 [M+H]⁺.

¹³C NMR (125 MHz, CDCl₃) [δ/ppm] 182.8, 178.6, 103.0, 94.9, 83.9, 78.0, 77.8, 78.0, 73.8, 73.6, 72.9, 72.8, 72.8, 67.9, 65.6, 60.0, 57.2, 56.7, 49.5, 47.5, 45.2, 42.0, 41.8, 36.4, 34.7, 30.9, 29.7, 27.2, 22.7, 21.8, 21.6, 20.9, 20.9, 18.2, 16.0, 14.9, 14.0, 11.0, 9.5.

Intermediate 13

### 3′-N-Demethyl-6-O-methyl-9-deoxo-8a-methyl-8a-aza-8a-homoerythromycin A

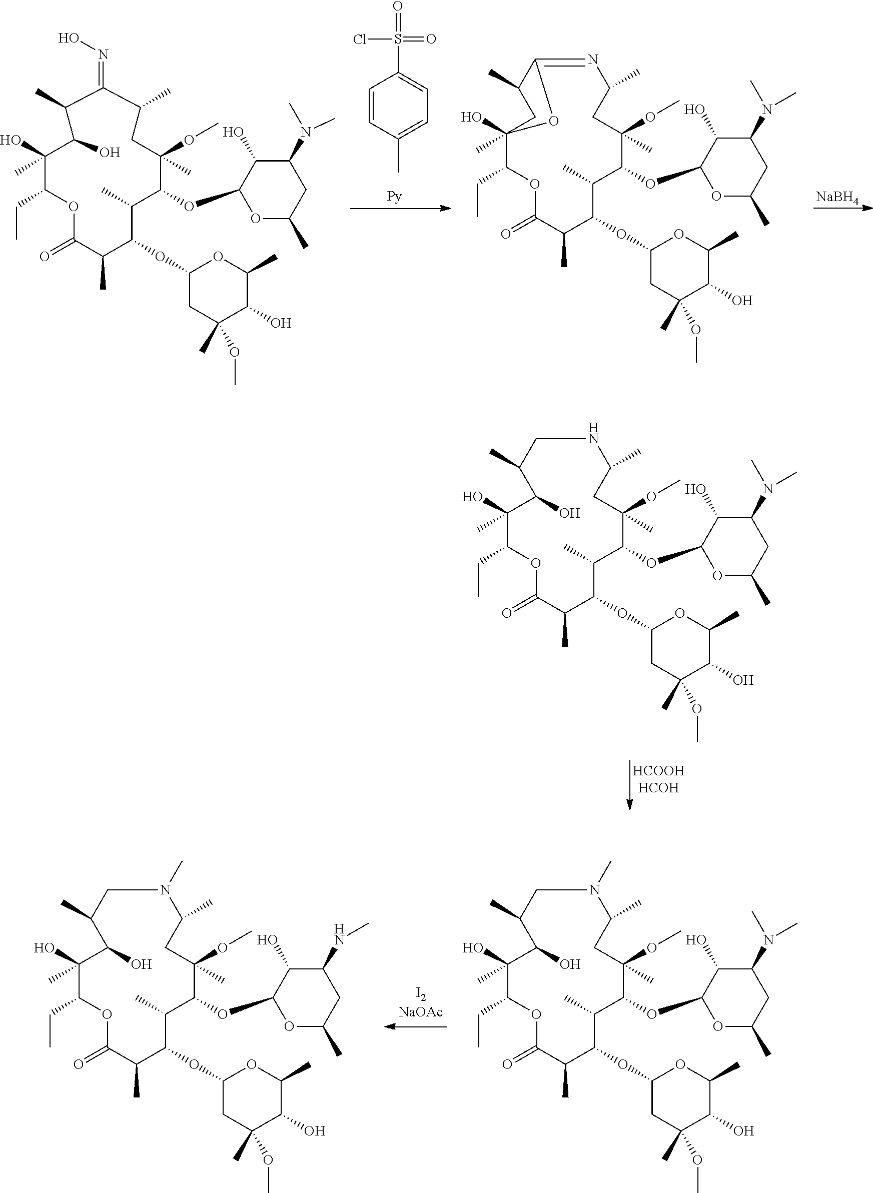

### Step a) 6-O-Methyl-erythromycin A 9,12-iminoether

A suspension of p-toluenesulfonyl chloride (14.99 g, 79 mmol) in diethyl ether (30 mL) was added rapidly into ice-cold solution of 6-O-methyl-erythromycin A 9(Z)-oxime (20 g, 26.2 mmol) in dry pyridine (150 mL). The resulting yellowish solution was stirred at 0° C. for 2 hours, then diluted with CH₂Cl₂ (50 mL) and water (100 mL), and pH adjusted to 9.5 (using 10 N NaOH). Layers were separated and aqueous layer was extracted with CH₂Cl₂ (2×40 mL). The combined organic layers were dried over anhydrous Na₂SO₄, filtered and evaporated to afford yellow solid which was dissolved in a mixture of EtOAc/n-hexane (1:2). Evaporation of the solvent afforded title product (18.5 g) as yellowish solid, that was used in next step without purification.

### Step b) 6-O-Methyl-9-deoxo-8a-aza-8a-homoerythromycin A

A solution of 6-O-methyl-erythromycin A 9,12-iminoether from Step a (18.5 g, 24.83 mmol) in ethylene glycol (150 mL) was cooled in ice bath and stirred under N₂ stream. Then, sodium borohydride (2.349 g, 62.1 mmol) was added in two portions within 1 hour. Following the borohydride addition, resulting suspension was stirred for 1.5 hour in an ice bath, and then overnight at room temperature. Reaction mixture was diluted with water (100 mL) and CH₂Cl₂ (100 mL), stirred vigorously, and layers were separated. Aqueous layer was further extracted with CH₂Cl₂ (2×40 mL). The combined organic layers were evaporated and dissolved in a mixture of 2-propanol (100 mL) and hexane (40 mL). Solution was evaporated to afford title product (15.2 g) as a white foam that was used in next step without purification; MS (ES+) m/z: 749.70 [M+H]⁺.

¹³C NMR (125 MHz, CDCl₃) [δ/ppm] 178.9, 103.1, 95.2, 79.8, 79.0, 78.2, 78.0, 75.8, 75.1, 72.6, 70.9, 68.7, 68.2, 65.4, 51.6, 50.8, 49.4, 44.8, 42.9, 42.2, 40.3, 34.6, 33.8, 28.9, 22.0, 21.5, 21.4, 21.1, 20.9, 18.4, 16.2, 15.0, 12.3, 11.2, 9.1.

### Step c) 6-O-Methyl-9-deoxo-8a-methyl-8a-aza-8a-homoerythromycin A

To a solution of 6-O-methyl-9-deoxo-8a-aza-8a-homoerythromycin A from Step b (10 g, 13.35 mmol) in chloroform (60 mL), formaldehyde (1.124 mL, 14.69 mmol) and formic acid (1.434 mL, 37.4 mmol) were added. The resulting solution was stirred at reflux temperature overnight, diluted with CH₂Cl₂ (150 mL) and water (120 mL), and stirred vigorously for a few minutes. The CH₂Cl₂ layer was discarded and fresh CH₂Cl₂ was added (100 mL). The pH of the resulting mixture was adjusted to 9.5 (aqueous NH₄OH). Layers were separated and aqueous layer was additionally extracted with CH₂Cl₂ (2×40 mL). The combined organic layers were dried over anhydrous Na₂SO₄, filtered, and evaporated to afford foam that was further dissolved in warm ethanol (24 mL) and water (12 mL), and stirred at RT for 5 minutes to afford white precipitate. The mixture was diluted with more water (12 mL), stirred, and cooled in ice bath, and then left in a refrigerator overnight. The mixture was filtered, collected solid was rinsed with a mixture of ethanol/water (12 mL; 3:1), and dried under reduced pressure to afford title product (4.8 g) as a white solid; MS (ES+) m/z: 763.80 [M+H]⁺.

¹³C NMR (125 MHz, CDCl₃) [δ/ppm] 102.6, 95.4, 79.5, 78.7, 77.9, 76.9, 76.2, 72.7, 70.9, 70.2, 68.8, 65.3, 62.8, 55.6, 50.7, 49.3, 45.6, 41.5, 40.5, 40.2, 34.8, 32.0, 28.8, 22.2, 21.4, 21.2, 18.3, 16.3, 15.0, 13.9, 13.5, 11.3, 9.7.

### Step d) 3′-N-Demethyl-6-O-methyl-9-deoxo-8a-methyl-8a-aza-8a-homoerythromycin A

To a stirred solution of 6-O-methyl-9-deoxo-8a-methyl-8a-aza-8a-homoerythromycin A from Step c (2 g, 2.62 mmol) and sodium acetate (1 g, 12.19 mmol) in MeOH (100 mL), solid iodine (0.8 g, 3.1 mmol) was added. The reaction mixture was irradiated with 500 W halogen lamp for 30 minutes. The solvent was evaporated and residue dissolved in CH₂Cl₂ (50 mL) and H₂O (50 mL). The pH of the mixture was adjusted to 9.8 (aqueous NH₄OH). Layers were separated and organic layer washed with saturated aqueous Na₂SO₃ solution (50 mL). The organic layer was evaporated and residue purified by Biotage SP1 system (25 g cartridge, using CH₂Cl₂-MeOH/CH₂Cl₂/NH₄OH (7.5:90:0.75)) to afford title product (120 mg); MS (ES+) m/z: 749.79 [M+H]⁺.

¹³C NMR (125 MHz, CDCl₃) [δ/ppm] 95.6, 79.5, 77.9, 77.1, 76.7, 74.9, 72.7, 68.7, 65.6, 60.2, 55.5, 50.4, 49.3, 45.5, 40.7, 40.6, 37.4, 34.8, 33.3, 32.0, 21.5, 21.1, 18.3, 16.2, 13.7, 11.3, 10.0.

### Examples 1 to 48

### Example 1

### N′-Benzyl-2′-O,3′-N-(carbonimidoyl)-3′-N-demethyl-9-deoxo-9a-methyl-9a-aza-9a-homoerythromycin A

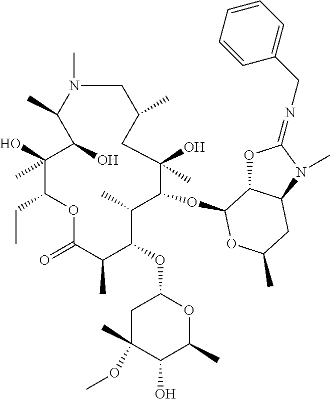

Method A:

A mixture of Intermediate 3 (0.47 g, 0.64 mmol), benzyl isothiocyanate (0.34 mL, 2.0 mmol), TEA (0.3 mL, 4.0 mmol) in acetonitrile (25 mL) was shaken in the vial at 60° C. for 3 hours. Then, EDC (0.5 g, 2.6 mmol) was added and shaking continued for 3 days at 60° C. Additional amount of EDC (0.05 g, 0.26 mmol) was added and shaken overnight at 60° C. Solvent was evaporated and the residue purified on the Flashmaster personal-solid phase extraction techniques (SPE 30 g, using MeOH/CH₂Cl₂/NH₄OH (4.5:90:0.25)) to afford title product (250 mg) as a single isomer.

HRMS (ES+) theoret. [M+H]⁺ C₄₅H₇₆N₃O₁₂ 850.5429, determined 850.5444 [M+H]⁺.

¹³C NMR (125 MHz, CDCl₃) [δ/ppm] 178.7, 157.0, 141.6, 128.1, 127.5, 126.4, 99.8, 95.2, 84.8, 80.6, 78.8, 74.1, 73.3, 78.0, 77.6, 73.1, 74.2, 70.1, 70.0, 65.8, 62.9, 62.4, 49.8, 49.4, 45.1, 42.0, 41.2, 36.4, 36.4, 34.8, 32.1, 27.3, 26.7, 21.9, 21.6, 21.1, 20.8, 18.1, 15.1, 16.2, 11.2, 8.7, 7.4.

Method B:

A mixture of Intermediate 3 (0.5 g, 0.68 mmol), benzyl isothiocyanate (0.27 mL, 1.6 mmol), TEA (0.3 mL, 4.0 mmol), and P-Mukaiyama reagent (0.85 g, 1.18 mmol/g, 1.0 mmol) in acetonitrile (25 mL) was shaken in the vial at 60° C. overnight. Then an additional amount of P-Mukaiyama reagent (0.16 g, 0.19 mmol) was added and shaken in the vial at 60° C. for 4 hours. Solvent was evaporated and the residue purified by the Flashmaster personal-solid phase extraction techniques (SPE 25 g using MeOH/CH₂Cl₂/NH₄OH (4.5:90:0.25)) to afford title product (380 mg) as a single isomer having identical analytical data as product from Method A.

### Example 2

### 2′-O,3′-N-(Carbonimidoyl)-3′-N-demethyl-9-deoxo-9a-methyl-N′-(1-naphthyl)-9a-aza-9a-homoerythromycin A

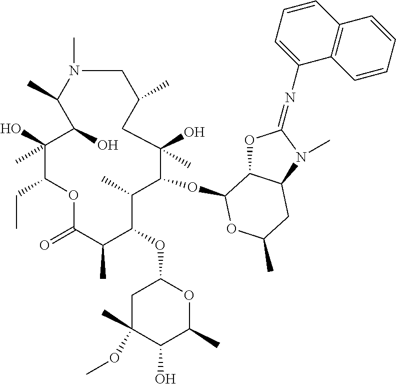

A mixture of Intermediate 3 (0.47 g, 0.64 mmol), 1-naphthyl isothiocyanate (0.37 g, 2.0 mmol), TEA (0.3 mL, 4.0 mmol) in acetonitrile (25 mL) was shaken in the vial at 60° C. for 1 hour. Then EDC (0.6 g, 3.1 mmol) was added and shaking continued overnight at 60° C. Solvent was evaporated and the residue purified on the Flashmaster personal-solid phase extraction techniques (SPE 30 g, using MeOH/CH₂Cl₂/NH₄OH (4.5:90:0.25)) to afford crude product (600 mg) which was further purified by Flashmaster personal-solid phase extraction techniques (SPE 30 g, using 1.5% MeOH/CH₂Cl₂-MeOH/CH₂Cl₂/NH₄OH (3:90:0.17)) to afford title product (245 mg) as a single isomer.

HRMS (ES+) theoret. [M+H]⁺ C₄₈H₇₆N₃O₁₂ 886.5429, determined 886.5451 [M+H]⁺.

¹³C NMR (125 MHz, CDCl₃) [δ/ppm] 178.6, 154.8, 143.0, 134.2, 129.6, 127.5, 125.6, 125.5, 124.7, 124.3, 122.3, 117.9, 99.9, 95.2, 84.8, 80.5, 78.5, 78.0, 78.0, 74.2, 73.2, 73.2, 74.2, 70.2, 70.2, 65.8, 62.6, 62.6, 49.6, 45.1, 41.9, 41.3, 36.5, 36.4, 34.8, 31.8, 27.3, 26.6, 22.4, 21.6, 21.1, 20.8, 18.1, 15.0, 16.2, 11.2, 8.3, 7.1.

It will be appreciated by the person skill in the art that the title compound may be also prepared from Intermediate 5 in a similar manner to that described in Example 5 hereinbelow.

### Example 3

### 2′-O,3′-N-(Carbonimidoyl)-3′-N-demethyl-9-deoxo-N′-isopropyl-9a-methyl-9a-aza-9a-homoerythromycin A

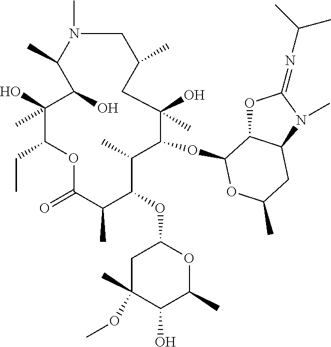

A mixture of Intermediate 3 (0.47 g, 0.64 mmol), isopropyl isothiocyanate (0.22 mL, 2.0 mmol), TEA (0.3 mL, 4.0 mmol) in acetonitrile (25 mL) was shaken in the vial at 60° C. for 1 hour. Then, EDC (0.6 g, 3.1 mmol) was added and shaking continued for 2 days at 60° C. Solvent was evaporated and the residue purified on the Flashmaster personal-solid phase extraction techniques (SPE 25 g, using MeOH/CH₂Cl₂/NH₄OH (4.5:90:0,25)) to afford title product (290 mg) as a single isomer.

HRMS (ES+) theoret. [M+H]⁺ C₄₁H₇₆N₃O₁₂ 802.5429, determined 802.5444 [M+H]⁺.

¹³C NMR (125 MHz, CDCl₃) [δ/ppm] 178.7, 155.9, 99.9, 95.3, 84.9, 80.4, 78.8, 77.6, 77.5, 74.3, 73.3, 73.2, 74.2, 70.2, 70.1, 65.8, 62.9, 62.4, 49.4, 45.1, 41.9, 41.0, 36.5, 36.4, 34.8, 32.3, 27.3, 26.7, 21.9, 21.6, 21.1, 20.8, 18.1, 15.2, 16.2, 11.2, 8.5, 7.3.

### Example 4

### 2′-O,3′-N-(Carbonimidoyl)-3′-N-demethyl-9-deoxo-N′-[3-(diethylamino)propyl]-9a-methyl-9a-aza-9a-homoerythromycin A

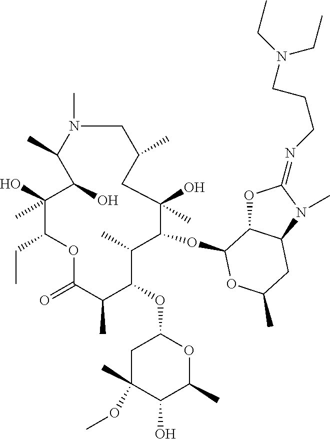

A mixture of Intermediate 3 (0.47 g, 0.64 mmol), 3-(diethylamino)propyl isothiocyanate (0.36 mL, 2.0 mmol), TEA (0.3 mL, 4.0 mmol) in acetonitrile (25 mL) was shaken in the vial at 60° C. for 2 hours. Then, EDC (0.6 g, 3.1 mmol) was added and shaking continued overnight at 60° C. Additional portion of EDC (0.1 g, 0.31 mmol) was added and shaking continued overnight at 60° C. Solvent was evaporated and the residue purified by the Flashmaster personal-solid phase extraction techniques (SPE 30 g, using 1.5% MeOH/CH₂Cl₂-MeOH/CH₂Cl₂/NH₄OH (4.5:90:0.25)) to afford title product (90 mg) as a single isomer.

HRMS (ES+) theoret. [M+H]⁺ C₄₅H₈₅N₄O₁₂ 873.6164, determined 873.6198 [M+H]⁺.

¹³C NMR (125 MHz, CDCl₃) [δ/ppm] 179.3, 157.0, 100.1, 95.4, 84.8, 80.9, 78.8, 78.3, 78.0, 74.2, 73.7, 73.6, 74.5, 70.5, 70.4, 66.2, 63.2, 62.9, 49.9, 49.9, 47.1, 45.6, 43.7, 41.9, 41.9, 36.8, 36.6, 35.1, 32.3, 27.7, 27.1, 25.6, 22.3, 21.9, 21.1, 21.1, 18.4, 16.6, 15.2, 11.5, 9.6, 8.7, 7.5.

### Example 5

### N′-Benzyl-2′-O,3′-N-(carbonimidoyl)-3′-N-demethyl-6-O-methyl-9a-aza-9a-homoerythromycin A

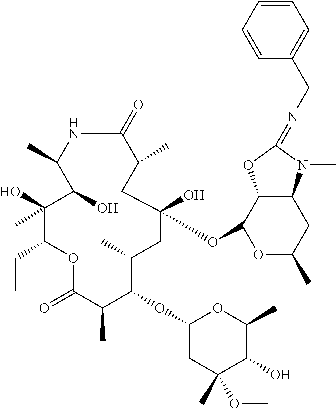

To a solution of Intermediate 4 (0.2 g, 0.22 mmol) in CHCl₃ (6 mL), EDC (85.4 mg, 0.44 mmol) was added. The reaction mixture was stirred at room temperature for 24 hours and then extracted with saturated aqueous NaHCO₃ (3×10 mL), brine (2×10 mL), and water (2×10 mL) and dried over K₂CO₃. After evaporation of the solvent crude product was purified by the Flashmaster II-solid phase extraction techniques (SPE 10 g, using gradient solvent system 97-96% DCM/(MeOH:NH₄OH=9:1.5) and 10 mL/min flow rate) to give title product (140 mg) as a single isomer.

HRMS (ES+) theoret. [M+H]⁺ C₄₅H₇₄N₃O₁₃ 864.5184, determined 864.5184 [M+H]⁺.

¹³C NMR (125 MHz, CDCl₃) [δ/ppm] 179.6, 177.4, 157.4, 138.1, 128.2, 127.7, 126.4, 99.5, 95.8, 81.0, 80.2, 79.1, 78.6, 77.8, 77.0, 74.3, 73.1, 73.0, 70.2, 66.0, 62.8, 51.6, 49.5, 49.5, 45.5, 44.6, 40.8, 40.0, 36.4, 35.6, 34.8, 32.1, 21.5, 21.0, 20.8, 20.6, 19.5, 18.3, 16.2, 15.4, 14.0, 11.2, 8.9.

### Example 6

### 2′-O,3′-N-(Carbonimidoyl)-3′-N-demethyl-6-O-methyl-N′-(1-naphthyl)-9a-aza-9a-homoerythromycin A

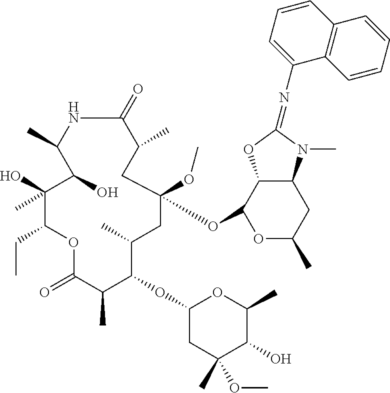

A mixture of Intermediate 1 (0.48 g, 0.64 mmol), 1-naphthyl isothiocyanate (0.37 g, 2.0 mmol), TEA (0.3 mL, 4.0 mmol) in acetonitrile (25 mL) was shaken in the vial at 60° C. for 2 hours. Then, EDC (0.6 g, 3.1 mmol) was added and shaking continued overnight at 60° C. Solvent was evaporated and the residue purified by the Flashmaster personal-solid phase extraction techniques (SPE 30 g, using 1.5% MeOH/CH₂Cl₂-MeOH/CH₂Cl₂/NH₄OH (3:90:0.17)) to afford title product (305 mg) as a single isomer.

HRMS (ES+) theoret. [M+H]⁺ C₄₈H₇₄N₃O₁₃ 900.5222, determined 900.5248 [M+H]⁺.

¹³C NMR (125 MHz, CDCl₃) [δ/ppm] 179.4, 176.9, 154.5, 141.8, 133.9, 129.4, 127.3, 125.4, 124.9, 124.3, 123.7, 122.3, 117.8, 99.4, 95.4, 80.2, 79.9, 78.7, 78.2, 77.5, 76.6, 73.8, 72.7, 72.5, 69.9, 65.5, 62.0, 51.1, 49.2, 44.9, 44.2, 40.4, 39.6, 36.1, 35.2, 34.4, 31.5, 21.2, 20.4, 20.5, 20.0, 19.0, 17.8, 15.8, 14.9, 13.6, 10.8, 8.1.

### Example 7

### 2′-O,3′-N-(Carbonimidoyl)-3′-N-demethyl-N′-isopropyl-6-O-methyl-9a-aza-9a-homoerythromycin A

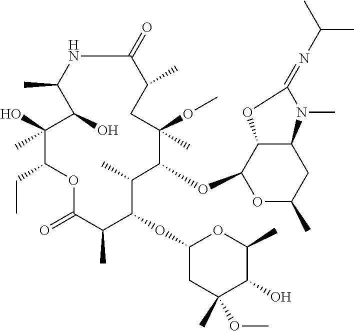

A mixture of Intermediate 1 (0.48 g, 0.64 mmol), isopropyl isothiocyanate (0.22 mL, 2.0 mmol), TEA (0.3 mL, 4.0 mmol) in acetonitrile (25 mL) was shaken in the vial at 60° C. for 2 hours. Then, EDC (0.6 g, 3.1 mmol) was added and shaking continued for 24 hours at 60° C. Solvent was evaporated and the residue purified by the Flashmaster personal-solid phase extraction techniques (SPE 25 g, using 1.5% MeOH/CH₂Cl₂-MeOH/CH₂Cl₂/NH₄OH (4.5:90:0.25)) to afford title product (290 mg) as a single isomer.

HRMS (ES+) theoret. [M+H]⁺ C₄₁H₇₄N₃O₁₃ 816.5222, determined 816.5231 [M+H]⁺.

¹³C NMR (125 MHz, CDCl₃) [δ/ppm] 179.3, 177.0, 154.6, 99.5, 95.4, 79.6, 79.8, 78.8, 78.2, 77.5, 76.7, 73.8, 72.6, 72.6, 69.8, 65.5, 62.4, 51.2, 49.0, 46.3, 45.0, 44.2, 40.3, 39.6, 36.0, 35.1, 34.4, 31.9, 24.1, 24.1, 21.1, 20.4, 20.5, 19.9, 19.0, 17.8, 15.7, 15.1, 13.7, 10.8, 8.2.

### Example 8

### 2′-O,3′-N-(Carbonimidoyl)-3′-N-demethyl-N′-[3-(diethylamino)propyl]-6-O-methyl-9a-aza-9a-homoerythromycin A

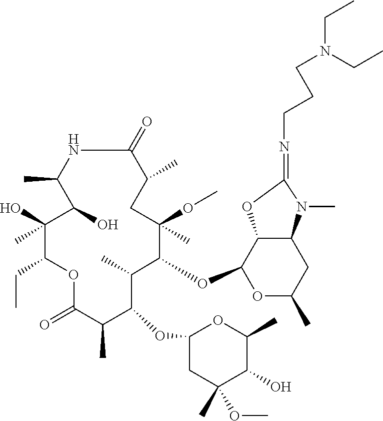

A mixture of Intermediate 1 (0.48 g, 0.64 mmol), 3-(diethylamino)propyl isothiocyanate (0.37 mL, 2.0 mmol), TEA (0.3 mL, 4.0 mmol) in acetonitrile (25 mL) was shaken in the vial at 60° C. for 2 hours. Then, EDC (0.6 g, 3.1 mmol) was added and shaking continued for 2 days at 60° C. Solvent was evaporated and the residue purified by the Flashmaster personal-solid phase extraction techniques (SPE 25 g, using 1.5% MeOH/CH₂Cl₂-MeOH/CH₂Cl₂/NH₄OH (9:90:0.5)) to afford crude product which was additionally purified on Biotage SP1 system (10 g cartridge, using CH₂Cl₂-MeOH/CH₂Cl₂/NH₄OH (7.5:90:0.75) to afford title product (105 mg) as a single isomer.

HRMS (ES+) theoret. [M+H]⁺ C₄₅H₈₃N₄O₁₃ 887.5957, determined 887.5925 [M+H]⁺.

¹³C NMR (125 MHz, CDCl₃) [δ/ppm] 179.6, 177.3, 155.7, 99.8, 95.6, 79.9, 79.8, 79.0, 78.4, 77.8, 76.7, 74.1, 72.9, 72.8, 70.0, 65.8, 62.6, 51.4, 50.5, 49.4, 46.8, 45.4, 44.8, 44.5, 40.7, 39.9, 36.4, 35.5, 34.7, 32.0, 28.3, 21.4, 20.8, 20.6, 20.3, 19.3, 18.1, 16.0, 15.2, 13.9, 11.6, 11.1, 8.5.

### Example 9

### N′-Benzyl-2′-O,3′-N-(carbonimidoyl)-3′-N-demethyl-6-O-methyl-erythromycin A

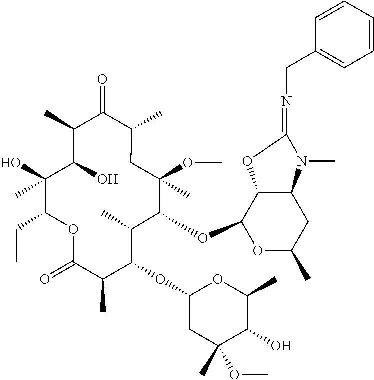

A mixture of Intermediate 2 (0.47 g, 0.64 mmol), benzyl isothiocyanate (0.34 g, 2.0 mmol), TEA (0.3 mL, 4.0 mmol) in acetonitrile (25 mL) was shaken in the vial at 60° C. for 2 hours. Then, EDC (0.6 g, 3.1 mmol) was added and shaking continued for 2 days at 60° C. Solvent was evaporated and the residue purified by the Flashmaster personal-solid phase extraction techniques (SPE 25 g, using 1.5% MeOH/CH₂Cl₂-MeOH/CH₂Cl₂/NH₄OH (4.5:90:0.25)) to afford title product (370 mg) as a single isomer.

HRMS (ES+) theoret. [M+H]⁺ C₄₅H₇₃N₂O₁₃ 849.5113, determined 849.5133 [M+H]⁺.

¹³C NMR (125 MHz, CDCl₃) [δ/ppm] 221.2, 176.0, 156.9, 142.2, 128.5, 127.8, 126.6, 100.0, 96.7, 82.1, 80.9, 79.2, 78.4, 78.2, 77.1, 74.6, 73.2, 70.6, 69.5, 66.3, 63.0, 51.1, 50.4, 49.8, 45.7, 45.3, 39.4, 38.8, 37.7, 36.8, 35.3, 32.5, 21.8, 21.4, 20.0, 21.3, 18.7, 18.4, 16.5, 16.3, 12.7, 10.9, 9.1.

### Example 10

### 2′-O,3′-N-(Carbonimidoyl)-3′-N-demethyl-6-O-methyl-N′-(1-naphthyl)-erythromycin A

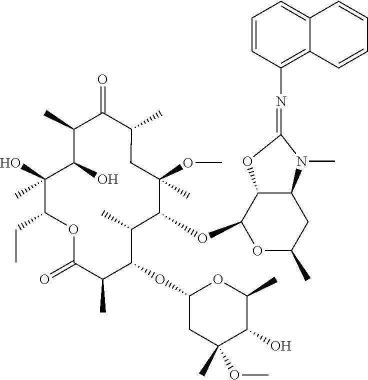

A mixture of Intermediate 2 (0.47 g, 0.64 mmol), 1-naphthyl isothiocyanate (0.37 g, 2.0 mmol), TEA (0.3 mL, 4.0 mmol) in acetonitrile (25 mL) was shaken in the vial at 60° C. for 2 hours. Then, EDC (0.6 g, 3.1 mmol) was added and shaking continued overnight at 60° C. Solvent was evaporated and the residue purified by the Flashmaster personal-solid phase extraction techniques (SPE 30 g, using 1.5% MeOH/CH₂Cl₂-500 ml MeOH/CH₂Cl₂/NH₄OH (2.25:90:0.125)) to afford title product (400 mg) as a single isomer.

HRMS (ES+) theoret. [M+H]⁺ C₄₈H₇₃N₂O₁₃ 885,5113, determined 885.5147 [M+H]⁺.

¹³C NMR (125 MHz, CDCl₃) [δ/ppm] 220.9, 175.7, 154.8, 142.4, 134.2, 129.7, 127.6, 125.6, 125.6, 124.8, 124.2, 122.6, 118.1, 99.7, 96.4, 81.9, 80.7, 78.9, 78.0, 77.8, 76.7, 74.2, 73.0, 70.3, 69.1, 65.9, 62.4, 50.6, 49.6, 45.3, 44.9, 39.1, 38.5, 36.4, 36.4, 34.9, 31.8, 21.5, 21.1, 20.9, 19.6, 18.3, 17.9, 16.0, 15.9, 12.3, 10.6, 8.5.

### Example 11

### 2′-O,3′-N-(Carbonimidoyl)-3′-N-demethyl-N′-isopropyl-6-O-methyl-erythromycin A

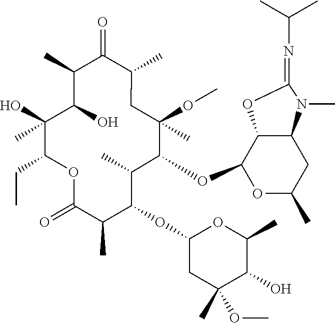

A mixture of Intermediate 2 (0.47 g, 0.64 mmol), isopropyl isothiocyanate (0.22 mL, 2.0 mmol), TEA (0.3 mL, 4.0 mmol) in acetonitrile (25 mL) was shaken in the vial at 60° C. for 2 hours. Then, EDC (0.6 g, 3.1 mmol) was added and shaking continued for 24 hours at 60° C. Solvent was evaporated and the residue purified by the Flashmaster personal-solid phase extraction techniques (SPE 25 g, using 1.5% MeOH/CH₂Cl₂-MeOH/CH₂Cl₂/NH₄OH (4.5:90:0.25)) to afford title product (325 mg) as a single isomer.

HRMS (ES+) theoret. [M+H]⁺ C₄₁H₇₃N₂O₁₃ 801.5113, determined 801.5138 [M+H]⁺.

¹³C NMR (125 MHz, CDCl₃) [δ/ppm] 220.4, 175.2, 155.7, 99.2, 96.0, 81.7, 80.6, 78.6, 77.6, 77.3, 76.4, 73.9, 72.6, 69.96, 68.7, 65.7, 62.3, 50.3, 49.1, 46.7, 44.9, 44.5, 38.6, 38.0, 36.9, 35.8, 34.6, 32.1, 23.7, 23.6, 21.1, 20.7, 20.5, 19.2, 17.9, 17.6, 15.7, 15.5, 12.7, 10.2, 8.2.

### Example 12

### 2′-O,3′-N-(Carbonimidoyl)-3′-N-demethyl-N′-[3-(diethylamino)propyl]-6-O-methyl-erythromycin A

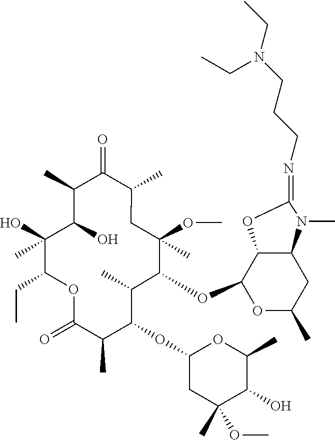

A mixture of Intermediate 2 (0.47 g, 0.64 mmol), 3-(diethylamino)propyl isothiocyanate (0.37 mL, 2.0 mmol), TEA (0.3 mL, 4.0 mmol) in acetonitrile (25 mL) was shaken in the vial at 60° C. for 2 hours. Then, EDC (0.6 g, 3.1 mmol) was added and shaking continued for 3 days at 60° C. Solvent was evaporated and the residue purified by the Flashmaster personal-solid phase extraction techniques (SPE 25 g, using 1.5% MeOH/CH₂Cl₂-MeOH/CH₂Cl₂/NH₄OH (9:90:0.5)) to afford crude product which was additionally purified on Biotage SP1 system (10 g cartridge, using CH₂Cl₂-MeOH/CH₂Cl₂/NH₄OH (7.5:90:0.75)) to afford title product (95 mg) as a single isomer.

HRMS (ES+) theoret. [M+H]⁺ C₄₅H₈₂N₃O₁₃ 872.5848, determined 872.5856 [M+H]⁺.

¹³C NMR (125 MHz, CDCl₃) [δ/ppm] 220.8, 175.5, 155.6, 99.7, 96.3, 81.6, 79.9, 78.8, 77.9, 77.7, 76.7, 74.1, 72.8, 70.1, 69.0, 65.8, 62.6, 50.6, 50.6, 49.4, 46.8, 45.5, 44.7, 44.7, 38.9, 38.4, 37.1, 36.3, 34.8, 32.0, 28.4, 21.4, 20.9, 20.8, 19.5, 18.3, 17.9, 15.9, 15.8, 12.2, 11.6, 10.5, 8.5.

### Example 13

### 2′-O,3′-N-(Carbonimidoyl)-3′-N-demethyl-N′-methyl-6-O-methyl-erythromycin A

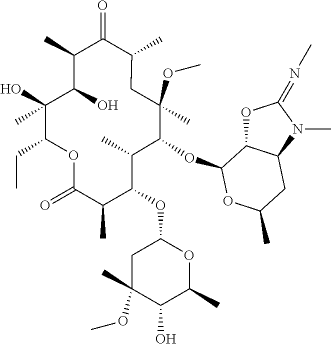

A mixture of Intermediate 2 (0.47 g, 0.64 mmol), methyl isothiocyanate (140 mg, 2.0 mmol), TEA (0.3 mL, 4.0 mmol) in acetonitrile (25 mL) was shaken in the vial at 60° C. for 2 hours. Then, EDC (0.6 g, 3.1 mmol) was added and shaking continued for 3 days at 60° C. Solvent was evaporated and the residue purified by the Biotage SP1 system (50 g cartridge, using CH₂Cl₂-MeOH/CH₂Cl₂/NH₄OH (7.5:90:0.75)) to afford crude product which was additionally purified using the same system and 50 g cartridge to afford title product (145 mg) as a single isomer.

HRMS (ES+) theoret. [M+H]⁺ C₃₉H₆₉N₂O₁₃ 773.4800, determined 773.4815 [M+H]⁺.

¹³C NMR (125 MHz, CDCl₃) [δ/ppm] 220.8, 175.5, 156.9, 99.7, 96.3, 81.7, 80.3, 78.9, 77.9, 77.7, 76.6, 74.2, 72.8, 70.1, 69.0, 65.9, 62.7, 50.6, 49.4, 45.2, 44.8, 38.9, 38.4, 37.2, 36.3, 34.8, 32.9, 31.9, 21.4, 20.9, 20.8, 19.5, 18.3, 17.9, 16.0, 15.8, 12.3, 10.5, 8.4.

### Example 14

### 2′-O,3′-N-(Carbonimidoyl)-3′-N-demethyl-N′-ethyl-6-O-methyl-erythromycin A

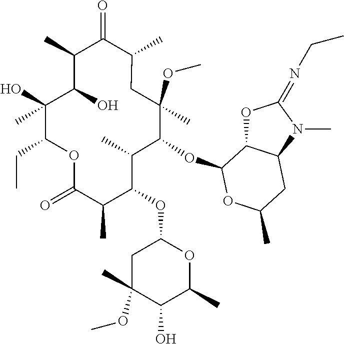

A mixture of Intermediate 2 (0.47 g, 0.64 mmol), ethyl isothiocyanate (0.168 mL, 2.0 mmol), TEA (0.355 mL, 4.6 mmol) in acetonitrile (25 mL) was shaken in the vial at 60° C. for 2 hours. Then, EDC (0.613 g, 3.2 mmol) was added and shaking continued 2 days at 60° C. The crude product (260 mg) which precipitated during the reaction was filtered off (260 mg) and purified by the Biotage SP1 system (10 g cartridge, using CH₂Cl₂-MeOH/CH₂Cl₂/NH₄OH (7.5:90:0.75)) to afford title product (250 mg) as a single isomer. The additional amount of the product was obtained by evaporation of the reaction mixture (780 mg) and crystallization from acetonitrile (55 mg).

HRMS (ES+) theoret. [M+H]⁺ C₄₀H₇₁N₂O₁₃ 787,4956, determined 787.4958 [M+H]⁺.

¹³C NMR (125 MHz, CDCl₃) [δ/ppm] 220.8, 175.5, 155.8, 99.8, 96.3, 81.8, 80.0, 78.8, 771, 77.7, 76.6, 74.2, 72.8, 70.1, 69.0, 65.8, 62.6, 50.6, 49.4, 45.2, 44.8, 38.9, 38.4, 37.2, 36.3, 34.8, 40.7, 32.0, 21.4, 20.9, 20.8, 19.5, 18.3, 17.9, 16.5, 16.0, 15.8, 12.3, 10.5, 8.5.

### Example 15

### 2′-O,3′-N-(Carbonimidoyl)-3′-N-demethyl-N′-ethyl-6-O-methyl-9a-aza-9a-homoerythromycin A

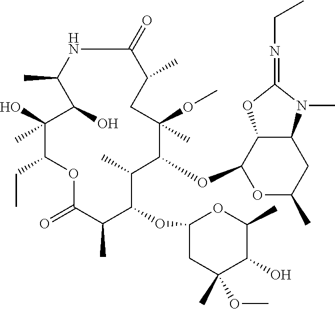

A mixture of Intermediate 1 (0.9 g, 1.2 mmol), ethyl isothiocyanate (0.305 mL, 3.6 mmol), TEA (0.665 mL, 8.9 mmol) in acetonitrile (50 mL) was shaken in the vial at 60° C. for 2 hours. Then, EDC (1.1 g, 5.7 mmol) was added and shaking continued overnight at 60° C. Solvent was evaporated and residue purified by the Biotage SP1 system (50 g cartridge, using CH₂Cl₂-MeOH/CH₂Cl₂/NH₄OH (7.5:90:0.75)) to afford crude product which was additionally purified using same system to afford title product (370 mg) as a single isomer.

HRMS (ES+) theoret. [M+H]⁺ C₄₀H₇₂N₃O₁₃ 802.5065, determined 802.5071 [M+H]⁺.

¹³C NMR (125 MHz, CDCl₃) [δ/ppm] 179.6, 177.3, 156.8, 99.5, 95.8, 80.7, 80.1, 79.0, 78.5, 77.7, 76.9, 74.1, 73.0, 72.9, 70.1, 65.9, 62.6, 51.5, 49.4, 40.4, 45.3, 44.5, 40.5, 39.9, 36.2, 35.4, 34.7, 32.0, 21.4, 20.6, 20.8, 20.2, 19.3, 18.1, 16.2, 16.0, 15.4, 14.0, 11.1, 8.5.

### Example 16

### 2′-O,3′-N-(Carbonimidoyl)-3′-N-demethyl-6-O-methyl-N′-(4-quinolyl)-9a-aza-9a-homoerythromycin A

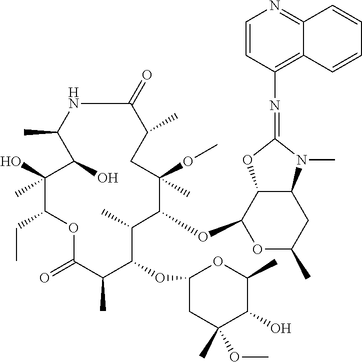

A mixture of Intermediate 1 (1 g, 1.33 mmol), 4-quinolyl isothiocyanate (0.283 g, 1.52 mmol) in DCM (50 mL) was stirred in the round-bottomed flask at room temperature for 24 hours. Then, EDC (0.768 g, 4.01 mmol) was added and stirring continued overnight at room temperature. The reaction mixture was extracted with saturated NaHCO₃, brine, and water and dried over Na₂SO₄. Solvent was evaporated to afford crude product (1.8 g) which was purified by the Flashmaster personal-solid phase extraction techniques (SPE 20 g, using solvent system gradient 100-95% CH₂Cl₂/(MeOH:NH₄OH=9:1.5)) to afford title product (410 mg). Precipitation of 400 mg of the obtained product from CH₂Cl₂ afforded title product (103 mg) as a single isomer.

MS (ES+) m/z: 901.66 [M+H]+.

¹³C NMR (125 MHz, CDCl₃) [δ/ppm] 179.6, 177.2, 156.0, 150.7, 150.5, 149.2, 128.9, 128.7, 125.8, 124.8, 123.9, 112.7, 99.4, 95.8, 80.1, 80.3, 79.0, 78.6, 77.7, 77.0, 74.1, 73.0, 72.9, 70.1, 65.9, 61.8, 51.5, 49.5, 45.3, 44.4, 40.4, 39.9, 36.2, 35.8, 34.7, 31.4, 21.4, 20.7, 20.6, 20.2, 19.2, 18.2, 16.0, 15.4, 13.9, 11.8, 8.6.

### Example 17

### 2′-O,3′-N-(Carbonimidoyl)-3′-N-demethyl-N′-(2,6-difluorophenyl)-6-O-methyl-9a-aza-9a-homoerythromycin A

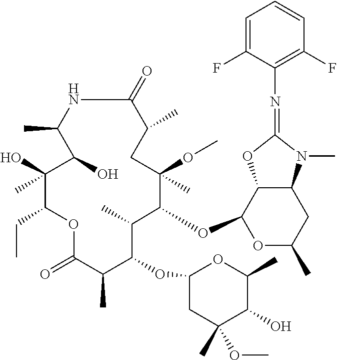

Method A:

A mixture of Intermediate 1 (0.6 g, 0.8 mmol), 2,6-difluorophenyl isothiocyanate (309 μL, 2.4 mmol) and TEA (444 μL, 3.2 mmol) in acetonitrile (30 mL) was shaken in the vial at 60° C. for 3 h. Then, EDC (764 mg, 4.0 mmol) was added and shaking continued for 2 days at 60° C. Then an additional amount of EDC (382 mg, 2.0 mmol) was added and shaking continued for 3 days at 60° C. Solvent was evaporated and the residue dissolved in CH₂Cl₂ (50 mL), water was added (50 mL) and pH of the resulting mixture adjusted from 6.0 to 6.5 using aqueous NH₄OH. To the organic layer water was added (50 mL) and pH adjusted to 5.0 using 1N HCl. Layers were separated and to the organic one water (50 mL) was added and pH of the mixture adjusted to 6.0, layers were separated and the organic one was dried over anhydrous Na₂SO₄, solvent was evaporated to afford crude product which was purified by the Biotage SP1 system (50 g cartridge, using CH₂Cl₂-MeOH/CH₂Cl₂/NH₄OH (15:90:1.5) to afford title product which was further purified by the Biotage SP1 system (10 g cartridge, using 2% DEA/EtOAc-hexane) to afford title product (50 mg) as a single isomer.

MS (ES+) m/z: 886.5 [M+H]⁺.

¹³C NMR (125 MHz, CDCl₃) [δ/ppm] 179.6, 177.2, 157.0, 156.8, 155.1, 124.8, 122.2, 110.7, 99.5, 95.7, 80.5, 80.2, 78.9, 78.4, 77.8, 77.2, 74.1, 73.9, 72.8, 70.1, 65.7, 62.6, 51.4, 49.4, 45.2, 44.4, 40.6, 39.8, 36.3, 35.5, 34.7, 31.5, 21.4, 20.8, 20.6, 20.2, 19.3, 18.1, 16.0, 15.4, 13.9, 11.1, 8.0.

Method B:

A mixture of Intermediate 1 (1.5 g, 2.0 mmol) and 2,6-difluorophenyl isothiocyanate (778 μL, 6.0 mmol) in acetonitrile (80 mL) was stirred at 60° C. for 2 hours. Then EDC (1.92 g, 10.0 mmol) was added and stirring continued for 2 days at 60° C. Solvent was evaporated, the residue dissolved in CH₂Cl₂ (80 mL), water (80 mL) was added, and pH adjusted to 5.2 using aqueous NH₄OH. Layers were separated and organic layer dried over anhydrous Na₂SO₄. Evaporation of the solvent afforded crude product which was purified by silica gel column chromatography, using CH₂Cl₂-MeOH/CH₂Cl₂/NH₄OH (7.5:90:0.75) to afford crude product which was further purified by silica gel column chromatography, using DEA/EtOAc/hexane (1:5:10) to afford title product (657 mg) as a single isomer having identical analytical data as product from Method A.

### Example 18

### 2′-O,3′-N-(Carbonimidoyl)-3′-N-demethyl-6-O-methyl-N′-(tert-butyl)-9a-aza-9a-homoerythromycin A

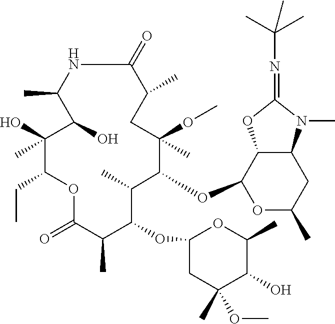

A mixture of Intermediate 1 (0.6 g, 0.8 mmol), tent-butyl isothiocyanate (1.5 ml, 12.2 mmol) and TEA (444 μL, 3.2 mmol) in acetonitrile (30 mL) was shaken in the vial at 60° C. for 2 h. Then an additional amount of tert-butyl isothiocyanate (1.2 ml, 9.7 mmol) was added and shaking continued overnight at 60° C. Then, EDC (768 mg, 4.0 mmol) was added and shaking continued overnight at 60° C. Solvent was evaporated and the residue dissolved in CH₂Cl₂ (50 mL), water (50 mL) was added and pH of the resulting mixture adjusted from 7.3 to 6.5 using 1N HCl. Layers were separated and organic one was evaporated. The residue was dissolved in EtOAc (50 mL), water was added (50 mL) and pH of the mixture adjusted to 4.0, layers were separated and to the aqueous one CH₂Cl₂ (50 mL) was added, pH of the resulting mixture adjusted to 7.6. Layers were separated and organic layer was dried over anhydrous Na₂SO₄ Solvent was evaporated to afford crude product that was purified by the Biotage SP1 system (10 g cartridge, using CH₂Cl₂-MeOH/CH₂Cl₂/NH₄OH (15:90:1.5) to afford title product that was further purified by the Biotage SP1 system (10 g cartridge, using 2% DEA/EtOAc-hexane) to afford title product (200 mg) which was dissolved in EtOAc (30 mL), water (30 m) was added and pH of the mixture adjusted to 4.0 using 1N HCl. Layers were separated and to the aqueous one CH₂Cl₂ (30 mL) was added, pH of the mixture adjusted to 8.4. Layers were separated and the organic was dried over anhydrous Na₂SO₄, solvent was evaporated to afford title product (100 mg) as a single isomer.

MS (ES+) m/z: 830.60 [M+H]⁺.

¹³C NMR (125 MHz, CDCl₃) [δ/ppm] 179.6, 177.4, 152.8, 99.7, 95.7, 79.9, 79.7, 79.1, 78.6, 77.8, 76.9, 74.1, 73.0, 72.9, 70.0, 65.8, 62.1, 51.9, 51.5, 49.3, 45.3, 44.5, 40.6, 39.8, 36.4, 35.3, 34.7, 32.6, 30.5, 21.4, 20.9, 20.6, 20.1, 19.2, 18.1, 16.0, 15.5, 14.0, 11.1, 9.2.

### Example 19

### 2′-O,3′-N-(Carbonimidoyl)-3′-N-demethyl-N′-methyl-6-O-methyl-9a-aza-9a-homoerythromycin A

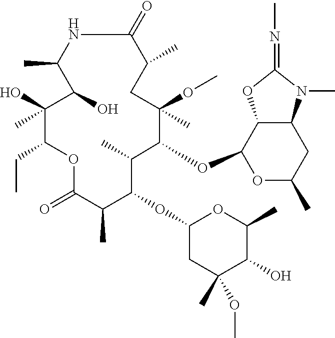

A mixture of Intermediate 1 (450 mg, 0.6 mmol), methyl isothiocyanate (132 mg, 1.8 mmol) and TEA (333 μL, 2.4 mmol) in acetonitrile (25 mL) was stirred at 60° C. for 2 h. Then EDC (574 mg, 3.0 mmol) was added and stirring continued at 60° C. overnight. Solvent was evaporated, the residue dissolved in CH₂Cl₂ (50 mL), water was added (50 mL), and pH adjusted to 4.1 (1N HCl). Layers were separated and aqueous layer additionally extracted with CH₂Cl₂ (3×30 mL). Combined organic layers were dried over anhydrous Na₂SO₄. Evaporation of the solvent afforded crude product which was purified by Waters Mass Directed Autopurification system using Waters XBridge C18, MS column (19×100 mm, 5 mm) with 17 mL/min flow rate (60% 10 mM NH₄HCO₃ pH=10.0 and 40% CH₃CN) to afford title product (55 mg) as a single isomer.

HRMS (ES+) theoret. [M+H]⁺ C₃₉H₇₀N₃O₁₃ 788.4909, determined: 788.4909 [M+H]⁺.

¹³C NMR (125 MHz, CDCl₃) [δ/ppm] 179.6, 177.3, 156.7, 99.7, 95.7, 79.9, 79.0, 78.5, 77.8, 76.9, 74.1, 72.9, 72.8, 70.1, 65.8, 62.7, 51.5, 49.4, 45.3, 44.5, 40.7, 39.9, 36.4, 35.5, 34.4, 33.1, 31.9, 21.4, 20.8, 20.6, 20.3, 19.3, 18.1, 16.0, 15.3, 13.9, 11.1, 8.4.

### Example 20

### 2′-O,3′-N-(Carbonimidoyl)-3′-N-demethyl-6-O-methyl-N′-[2-(4-morpholinyl)ethyl]-9a-aza-9a-homoerythromycin A

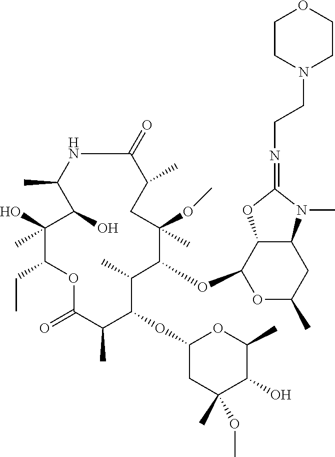

A mixture of Intermediate 1 (0.6 g, 0.8 mmol), 2-(4-morpholino)ethyl isothiocyanate (376 μL, 2.4 mmol) and TEA (725 μL, 3.2 mmol) in acetonitrile (30 mL) was stirred at 60° C. for 2 hours. Then EDC (768 mg, 4.01 mmol) was added and stirring continued at 60° C. for 2 days. Solvent was evaporated, residue dissolved in CH₂Cl₂ (50 mL), water (50 mL) was added, and pH adjusted to 6.5 (1N HCl). To the organic layer water (50 ml) was added and pH adjusted 4.0 (1N HCl). To the aqueous layer fresh CH₂Cl₂ (50 mL) was added and pH adjusted to 6.8 (eq. NH₄OH). Layers were separated and the organic one was dried over anhydrous Na₂SO₄, solvent was evaporated to afford crude product which was purified by Biotage SP1 system (10 g cartridge, using CH₂Cl₂-MeOH/CH₂Cl₂/NH₄OH (7.5:90:0.75) to afford title product (20 mg) as a single isomer.

HRMS (ES+) theoret. [M+H]⁺ C₄₄H₇₉N₄O₁₄: 887.5593, determined: 887.5606 [M+H]⁺.

¹³C NMR (125 MHz, CDCl₃) [δ/ppm] 179.6, 177.3, 156.2, 99.7, 95.6, 79.9, 79.9, 79.0, 78.5, 77.8, 76.9, 74.1, 72.9, 72.8, 70.0, 67.0, 65.8, 62.6, 60.0, 53.8, 51.5, 49.4, 45.3, 44.5, 43.5, 40.7, 39.9, 36.4, 35.6, 34.7, 31.9, 21.4, 20.8, 20.6, 20.3, 19.4, 18.2, 16.0, 15.3, 13.9, 11.1, 8.7.

### Example 21

### 2′-O,3′-N-(Carbonimidoyl)-3′-N-demethyl-N′-[3-(methyloxy)propyl]-6-O-methyl-9a-aza-9a-homoerythromycin A

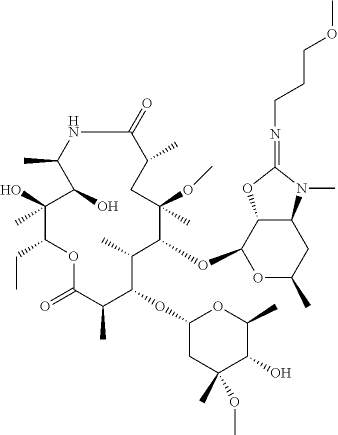

A mixture of Intermediate 1 (0.6 g, 0.8 mmol) and 3-metoxypropyl isothiocyanate (306 μL, 2.4 mmol) in acetonitrile (30 mL) was stirred at 60° C. for 2 hours. Then EDC (461 mg, 2.4 mmol) was added and stirring continued at 60° C. overnight. Solvent was evaporated and the residue dissolved in CH₂Cl₂ (40 mL), water (40 mL) was added, and pH of the resulting mixture adjusted to 6.5 (1N HCl). The organic layer was evaporated and the residue dissolved in EtOAc (50 mL), water (50 mL) was added, and pH of the resulting mixture adjusted to 4.0 (1N HCl). Layers were separated and aqueous one was additionally extracted with EtOAc (20 mL). To the aqueous layer CH₂Cl₂ (50 mL) was added and pH of the resulting mixture adjusted to 5.0 (aqueous NH₄OH). Than, aqueous layer was additionally extracted with CH₂Cl₂ (2×20 mL). Combined organic layers at pH 5.0 were dried over anhydrous Na₂SO₄. Evaporation of the solvent afforded crude product which was purified by Waters Mass Directed Autopurification system using Waters XBridge C18, MS column (19×100 mm, 5 mm) with 17 mL/min flow rate (linear gradients from 70% 10 mM NH₄HCO₃ pH=10.0 and 30% CH₃CN to 50% 10 mM NH₄HCO₃ pH=10.0 and 50% CH₃CN over 20 min) to afford title product as a single isomer (45 mg).

HRMS (ES+) theoret. [M+H]⁺ C₄₂H₇₆N₃O₁₄ 846.5327, determined 846.5327 [M+H]⁺.

¹³C NMR (125 MHz, CDCl₃) [δ/ppm] 179.6, 177.3, 155.9, 99.8, 95.7, 79.9, 79.8, 79.0, 78.5, 77.8, 76.9, 74.1, 72.9, 72.8, 71.1, 70.1, 65.8, 62.6, 58.4, 51.5, 49.4, 45.3, 44.5, 40.7, 39.9, 36.4, 35.5, 34.7, 32.0, 31.4, 31.1, 21.4, 20.8, 20.6, 20.3, 19.3, 18.1, 16.0, 15.3, 13.9, 11.1, 8.5.

### Example 22

### 2′-O,3′-N-(Carbonimidoyl)-3′-N-demethyl-N′-[3-(methylthio)propyl]-6-O-methyl-9a-aza-9a-homoerythromycin A

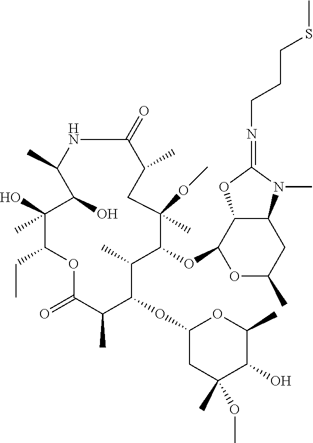

A mixture of Intermediate 1 (0.7 g, 0.94 mmol) and 3-(methylthio)propyl isothiocyanate (375 μL, 2.8 mmol) in acetonitrile (35 mL) was stirred at 60° C. for 2 hours. Then EDC (538 mg, 2.8 mmol) was added and stirring continued at 60° C. overnight. Solvent was evaporated, the residue dissolved in CH₂Cl₂ (40 mL), water (40 mL) was added, and pH of the resulting mixture adjusted to 6.5 (1N HCl). The organic layer was evaporated, residue dissolved in EtOAc (40 mL), water (40 mL) was added and pH of the resulting mixture adjusted to 4.0 (1N HCl). Layers were separated and to the aqueous one fresh CH₂Cl₂ (40 mL) was added, and pH adjusted to 5.0 (aq. NH₄OH). Layers were separated and organic layer dried over anhydrous Na₂SO₄. Evaporation of the solvent afforded crude product that was purified by Biotage SP1 system (10 g cartridge, using 2% DEA/EtOAc-hexane) to afford product that was precipitated from diethyl ether to afford title product as a single isomer (108 mg).

HRMS (ES+) theoret. [M+H]⁺ C₄₂H₇₆N₃O₁₃S 862.5099, determined 862.5081 [M+H⁺.

¹³C NMR (125 MHz, CDCl₃) [δ/ppm] 179.6, 177.3, 155.9, 99.8, 95.7, 79.8, 80.0, 79.0, 78.5, 77.8, 76.9, 74.1, 72.9, 72.8, 70.1, 65.8, 62.7, 51.5, 49.4, 45.5, 45.3, 44.5, 40.7, 39.9, 36.4, 35.5, 34.7, 32.0, 32.0, 31.1, 21.4, 20.8, 20.6, 20.3, 19.4, 18.1, 16.0, 15.4, 15.3, 13.9, 11.1, 8.6.

### Example 23

### 2′-O,3′-N-(Carbonimidoyl)-3′-N-demethyl-6-O-methyl-N′-[4-(methyloxy)phenyl]-9a-aza-9a-homoerythromycin A

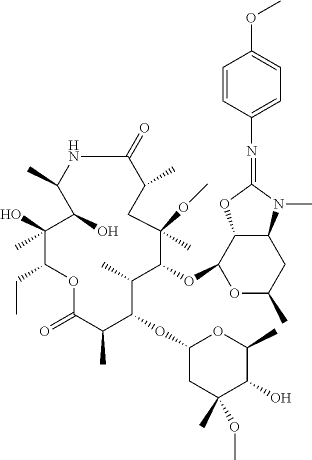

A mixture of Intermediate 1 (0.7 g, 0.94 mmol) and 4-methoxyphenyl isothiocyanate (392 μL, 2.8 mmol) in acetonitrile (35 mL) was stirred at 60° C. for 2 hours. Then EDC (538 mg, 2.8 mmol) was added and stirring continued at 60° C. overnight. Solvent was evaporated, the residue dissolved in CH₂Cl₂ (50 mL), and washed with water (50 mL). Organic layer was evaporated, the residue dissolved in EtOAc (50 mL), water (50 mL) was added, and pH of the resulting mixture adjusted to 4.0 (1N HCl). Layers were separated, and the organic layer was additionally washed with water (2×30 mL). After evaporation of the organic layer, the residue was dissolved in CH₂Cl₂ (50 mL), water (50 mL) was added, and pH of the resulting mixture adjusted to 6.6 (aqueous NH₄OH). Layers were separated and the organic layer evaporated to afford crude product which was first purified by Biotage SP1 system (10 g cartridge, using CH₂Cl₂-MeOH/CH₂Cl₂/NH₄OH (7.5:90:0.75)) and then precipitated from a mixture of acetone/water to afford title product as a single isomer (226 mg).

HRMS (ES+) theoret. [M+H]⁺ C₄₅H₇₄N₃O₁₄ 880.5171, determined 880.5200 [M+H]⁺.

¹³C NMR (125 MHz, CDCl₃) [δ/ppm] 179.7, 177.3, 155.1, 154.0, 139.3, 124.6, 113.6, 99.7, 95.7, 80.0, 80.3, 79.0, 78.5, 77.8, 76.9, 74.1, 72.9, 72.8, 70.1, 65.8, 62.1, 55.4, 51.5, 49.5, 45.3, 44.5, 40.7, 39.9, 36.4, 35.5, 34.7, 31.7, 21.4, 20.8, 20.6, 20.3, 19.3, 18.1, 16.0, 15.3, 13.9, 11.1, 8.7

### Example 24

### 2′-O,3′-N-(Carbonimidoyl)-3′-N-demethyl-6-O-methyl-N′-(tetrahydro-2-furanylmethyl)-9a-aza-9a-homoerythromycin A

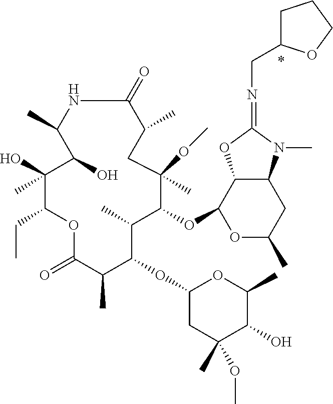

A mixture of Intermediate 1 (0.7 g, 0.94 mmol) and 2-tetrahydrofurfuryl isothiocyanate (359 μL, 2.8 mmol) in acetonitrile (35 mL) was stirred at 60° C. for 2 hours. Then EDC (538 mg, 2.8 mmol) was added and stirring continued at 60° C. overnight. Solvent was evaporated, the residue dissolved in CH₂Cl₂ (50 mL) and washed with water (50 mL). To the organic layer fresh water (50 mL) was added and pH of the resulting mixture adjusted to 4.0 (1N HCl). Layers were separated, and the organic one was dried over anhydrous Na₂SO₄. Evaporation of the solvent afforded crude product which was purified by Biotage SP1 system (10 g cartridge, using CH₂Cl₂-MeOH/CH₂Cl₂/NH₄OH (7.5:90:0.75)) to afford title product (260 mg) as a mixture of diastereomers in approximately 1:1 ratio.

HRMS (ES+) theoret. [M+H]⁺ C₄₃H₇₆N₃O₁₄ 858.5319, determined 858.5327 [M+H]⁺.

¹³C NMR (125 MHz, CDCl₃) [δ/ppm] 179.6, 177.3, 156.1, 99.7, 95.7, 79.9, 79.8, 79.6, 79.1, 78.5, 77.8, 76.8, 74.1, 72.6, 72.8, 70.1, 68.0, 65.8, 62.6, 51.5, 51.0, 49.4, 45.3, 44.5, 40.8, 39.9, 36.3, 35.4, 34.7, 31.9, 29.2, 21.4, 20.8, 20.6, 20.3, 19.3, 18.2, 16.0, 15.4, 13.9, 11.1, 8.5.

### Example 25

### 2′-O,3′-N-(Carbonimidoyl)-3′-N-demethyl-N′-(2-furanylmethyl)-6-O-methyl-9a-aza-9a-homoerythromycin A

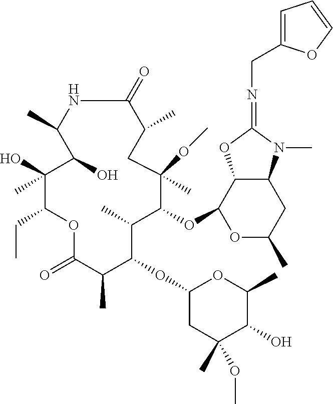

A mixture of Intermediate 1 (0.7 g, 0.94 mmol) and 2-furfuryl isothiocyanate (281 μL, 2.8 mmol) in acetonitrile (35 mL) was stirred at 60° C. for 2 hours. Then EDC (538 mg, 2.8 mmol) was added and stirring continued at 60° C. overnight. Solvent was evaporated, the residue dissolved in CH₂Cl₂ (40 mL), and washed with water (40 mL). Organic layer was evaporated, the residue dissolved in EtOAc (40 mL), water (50 mL) was added and pH of the resulting mixture adjusted to 4.0 (1N HCl). Layers were separated and the aqueous one additionally extracted with EtOAc (2×30 mL). To the aqueous layer CH₂Cl₂ (60 mL) was added, and pH of the resulting mixture adjusted to 9.3 (aqueous NH₄OH). Layers were separated and the organic one was dried over anhydrous Na₂SO₄. Evaporation of the solvent afforded crude product which was purified by Biotage SP1 system (10 g cartridge, using CH₂Cl₂-MeOH/CH₂Cl₂/NH₄OH (7.5:90:0.75)) to afford title product (160 mg) as a single isomer.

HRMS (ES+) theoret. [M+H]⁺ C₄₃H₇₂N₃O₁₄ 854.5014, determined 854.4996 [M+H]⁺.

¹³C NMR (125 MHz, CDCl₃) [δ/ppm] 179.6, 177.3, 156.7, 155.1, 141.1, 109.9, 105.5, 99.7, 95.7, 80.0, 80.1, 79.1, 78.5, 77.8, 77.1, 74.1, 72.9, 72.9, 70.1, 65.8, 62.6, 51.5, 49.4, 45.3, 44.5, 43.5, 40.6, 39.9, 36.3, 35.5, 34.7, 31.8, 21.4, 20.8, 20.6, 20.3, 19.3, 18.1, 16.0, 15.4, 13.9, 11.1, 8.6.

### Example 26

### 2′-O,3′-N-(Carbonimidoyl)-3′-N-demethyl-N′-isopropyl-(9S)-9-dihydroerythromycin A

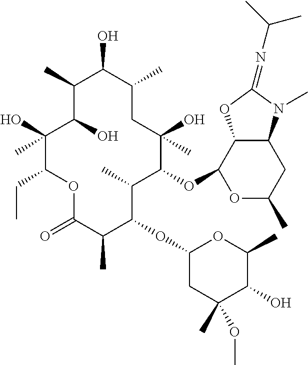

A mixture of Intermediate 6 (0.6 g, 0.83 mmol) and isopropyl isothiocyanate (266 μL, 2.49 mmol) in acetonitrile (30 mL) was stirred at 60° C. for 1 hour. Then EDC (478 mg, 2.49 mmol) was added and stirring continued at 60° C. during 30 h. Solvent was evaporated, the residue dissolved in CH₂Cl₂ (40 mL), water (40 mL) was added, and pH of the resulting mixture adjusted to 6.5 (1N HCl). Organic layer was separated and evaporated. The obtained residue was dissolved in EtOAc (40 mL), water (40 mL) was added, and pH of the resulting mixture adjusted to 4.0 (1N HCl). To the aqueous layer CH₂Cl₂ (40 mL) was added, and pH adjusted to 8.5 (aq. NH₄OH). Layers were separated and the organic one dried over anhydrous Na₂SO₄ Evaporation of the organic solvent afforded crude product which was purified by Biotage SP1 system (10 g cartridge, using CH₂Cl₂-MeOH/CH₂Cl₂/NH₄OH (7.5:90:0.75)) to afford title product (350 mg) as a single isomer.

HRMS (ES+) theoret. [M+H]⁺ C₄₀H₇₃N₂O₁₃ 789.5113, determined 789.5103 [M+H]⁺.

¹³C NMR (125 MHz, CDCl₃) [δ/ppm] 176.8, 154.5, 99.9, 96.3, 83.9, 82.9, 79.6, 79.9, 77.7, 77.5, 75.2, 74.5, 72.8, 70.5, 65.9, 62.6, 49.2, 46.5, 44.7, 39.9, 36.3, 35.5, 34.9, 34.3, 32.2, 31.7, 25.9, 24.6, 24.4, 21.8, 21.4, 20.8, 20.2, 17.9, 16.4, 15.6, 14.9, 10.9, 8.6.

### Example 27

### N′-Benzyl-2′-O,3′-N-(carbonimidoyl)-3′-N-demethyl-(9S)-9-dihydroerythromycin A

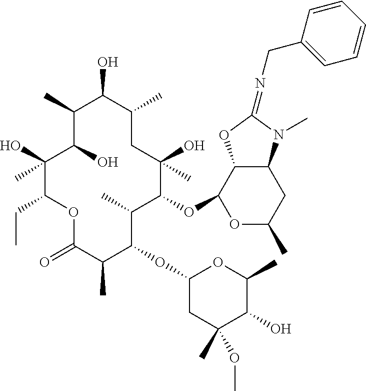

A mixture of Intermediate 6 (0.6 g, 0.83 mmol) and benzyl isothiocyanate (331 μL, 2.49 mmol) in acetonitrile (30 mL) was stirred at 60° C. for 3 hours. Then EDC (478 mg, 2.49 mmol) was added and stirring continued at 60° C. overnight. Solvent was evaporated, the residue dissolved in CH₂Cl₂ (40 mL), and washed with water (40 mL). To the organic layer water was added (40 mL), and pH adjusted to 6.5 (1N HCl). Layers were separated and the organic layer evaporated. Residue was dissolved in EtOAc (40 mL), water (40 mL) was added, and pH adjusted to 4.0 (1N HCl). Layers were separated and aqueous layer additionally extracted with EtOAc (2×30 mL). Combined organic layers were evaporated and purified by Biotage SP1 system (10 g cartridge, using CH₂Cl₂-MeOH/CH₂Cl₂/NH₄OH (7.5:90:0.75)) to afford title product (100 mg) as a single isomer.

HRMS (ES+) theoret. [M+H]⁺ C₄₄H₇₃N₂O₁₃ 837.5113, determined 837.5146 [M+H]⁺.

¹³C NMR (125 MHz, CDCl₃) [δ/ppm] 176.9, 156.3, 141.9, 127.9, 127.4, 126.0, 99.7, 96.2, 83.7, 83.1, 80.1, 79.7, 77.7, 77.6, 75.2, 74.4, 72.7, 70.4, 70.4, 65.9, 62.5, 50.1, 49.4, 44.7, 40.2, 36.3, 35.7, 34.9, 34.8, 32.0, 31.7, 26.0, 21.4, 21.4, 20.8, 20.1, 18.0, 16.4, 15.4, 14.8, 10.9, 8.8.

### Example 28

### 2′-O,3′-N-(Carbonimidoyl)-3′-N-demethyl-N′-ethyl-6-O-methyl-(9S)-9-dihydroerythromycin A

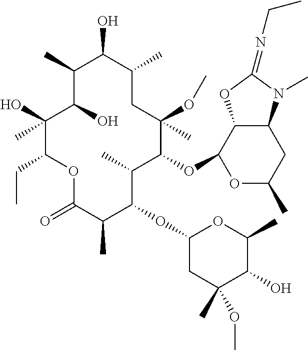

A mixture of Intermediate 7 (0.2 g, 0.27 mmol) and ethyl isothiocyanate (71 μL, 0.82 mmol) in acetonitrile (10 ml) was stirred at 60° C. for 1 hour. Then EDC (156 mg, 0.82 mmol) was added and stirring continued at 60° C. overnight. Solvent was evaporated and residue purified by Biotage SP1 system (10 g cartridge, using CH₂Cl₂-MeOH/CH₂Cl₂/NH₄OH (7.5:90:0.75)). The crude product was dissolved in CH₂Cl₂ (10 mL), water (10 mL) was added, and pH adjusted to 6.5 (1N HCl). Layers were separated and organic layer evaporated to afford title product (120 mg) as a single isomer.

HRMS (ES+) theoret. [M+H]⁺ C₄₀H₇₃N₂O₁₃ 789.5113, determined 789.5131 [M+H]⁺.

¹³C NMR (125 MHz, CDCl₃) [δ/ppm] 175.0, 157.7, 99.6, 96.7, 81.9, 79.8, 79.8, 79.4, 79.4, 77.6, 77.5, 74.8, 72.8, 70.9, 70.1, 65.9, 62.6, 50.7, 49.3, 45.0, 40.8, 37.9, 36.3, 35.0, 34.7, 34.4, 32.5, 32.0, 21.3, 21.3, 16.6, 16.6, 10.5, 8.7.

### Example 29

### 2′-O,3′-N-(Carbonimidoyl)-3′-N-demethyl-N′-isopropyl-6-O-methyl-(9S)-9-dihydroerythromycin A

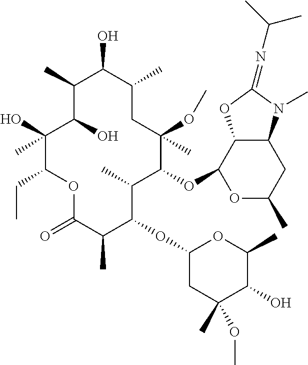

A mixture of Intermediate 7 (110 mg, 0.15 mmol) and isopropyl isothiocyanate (48 μl, 0.45 mmol) in acetonitrile (8 mL) was stirred at 60° C. for 1 hour. Then EDC (86 mg, 0.45 mmol) was added and stirring continued at 60° C. overnight. Solvent was evaporated and residue purified by the Biotage SP1 system (10 g cartridge, using CH₂Cl₂-MeOH/CH₂Cl₂/NH₄OH (7.5:90:0.75)) to afford title product (70 mg) as a single isomer.

HRMS (ES+) theoret. [M+H]⁺ C₄₁H₇₅N₂O₁₃ 803.5269, determined 803.5295 [M+H]⁺.

¹³C NMR (125 MHz, CDCl₃) [δ/ppm] 175.0, 154.5, 99.7, 96.7, 81.9, 79.8, 79.6, 79.5, 79.3, 77.7, 77.5, 74.8, 72.8, 70.9, 70.1, 65.8, 62.6, 50.7, 49.3, 46.5, 45.0, 37.9, 36.3, 35.0, 34.7, 34.4, 32.5, 32.3, 24.7, 24.4, 21.3, 21.3, 21.3, 20.9, 20.2, 18.2, 16.5, 16.8, 16.1, 10.5, 8.7.

### Example 30

### N′-Benzyl-2′-O,3′-N-(carbonimidoyl)-3′-N-demethyl-6-O-methyl-(9S)-9-dihydroerythromycin A

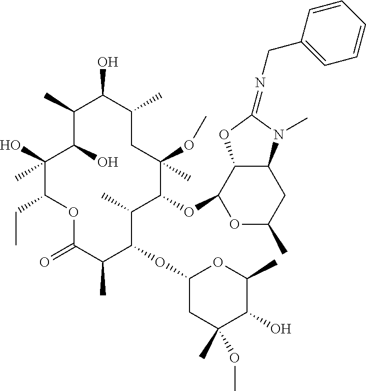

A mixture of Intermediate 7 (150 mg, 0.20 mmol) and benzyl isothiocyanate (81 μL, 0.61 mmol) in acetonitrile (8 mL) was stirred at 60° C. for 3 hours. Then EDC (117 mg, 0.61 mmol) was added and stirring continued at 60° C. overnight. Solvent was evaporated and residue purified by the Biotage SP1 system (10 g cartridge, using CH₂Cl₂-MeOH/CH₂Cl₂/NH₄OH (7.5:90:0.75)) to afford title product (115 mg) as a single isomer.

HRMS (ES+) theoret. [M+H]⁺ C₄₅H₇₅N₂O₁₃ 851.5269, determined 851.5289 [M+H]⁺.

¹³C NMR (125 MHz, CDCl₃) [δ/ppm] 175.0, 156.2, 141.9, 127.4, 127.9, 126.0, 99.5, 96.7, 81.9, 80.1, 79.8, 79.3, 79.4, 77.6, 77.5, 74.8, 72.7, 70.9, 70.1, 65.9, 62.5, 50.8, 50.0, 49.3, 45.0, 37.9, 36.3, 34., 34.7, 34.4, 32.4, 32.0, 21.3, 21.3, 21.3, 20.9, 20.2, 18.3, 16.5, 16.7, 16.1, 10.5, 8.8.

### Example 31

### 2′-O,3′-N-(Carbonimidoyl)-3′-N-demethyl-N′-isopropyl-erythromycin A

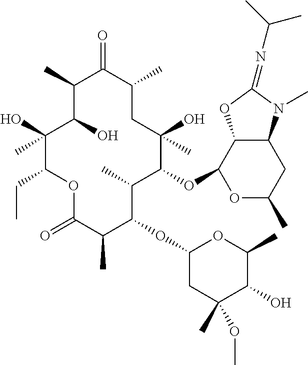

A mixture of Intermediate 8 (200 mg, 0.28 mmol) and isopropyl isothiocyanate (89 μL, 0.83 mmol) in acetonitrile (10 mL) was stirred at 60° C. for 1 hour. Then EDC (160 mg, 0.83 mmol) was added and stirring continued at 60° C. overnight. Solvent was evaporated and residue purified by Biotage SP1 system (10 g cartridge, using CH₂Cl₂-MeOH/CH₂Cl₂/NH₄OH (7.5:90:0.75)) to afford title product (65 mg) as a single isomer.

HRMS (ES+) theoret. [M+H]⁺ C₄₀H₇₁N₂O₁₃ 787.4931, determined 787.4956 [M+H]⁺.

¹³C NMR (125 MHz, CDCl₃) [δ/ppm] 221.9, 175.4, 154.5, 100.1, 96.4, 84.4, 80.3, 79.6, 77.7, 76.9, 74.5, 74.5, 72.7, 70.2, 68.8, 65.7, 62.8, 51.1, 49.3, 46.5, 44.6, 45.2, 38.4, 38.0, 37.6, 36.3, 34.9, 32.2, 26.8, 24.7, 24.4, 21.4, 20.9, 20.8, 18.2, 18.1, 16.2, 15.9, 12.0, 10.5, 8.5.

### Example 32

### 2′-O,3′-N-(Carbonimidoyl)-3′-N-demethyl-N′-ethyl-erythromycin A

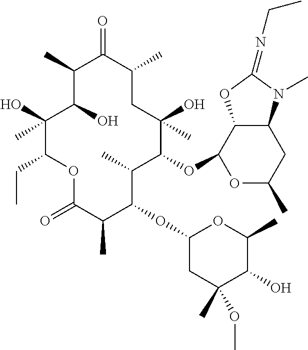

A mixture of Intermediate 8 (400 mg, 0.56 mmol), TEA (774 μL, 5.56 mmol), and ethyl isothiocyanate (146 μL, 1.67 mmol) in acetonitrile (50 mL) was stirred at 60° C. for 1 hour. Then EDC (320 mg, 1.67 mmol) was added and stirring continued at 60° C. overnight. Then, additional amount of EDC (745 mg, 3.89 mmol) was added and stirring continued at 60° C. for 20 hours. Solvent was evaporated, residue dissolved in CH₂Cl₂ (30 mL), water (30 mL) was added, and pH adjusted to 6.5 (1N HCl). Layers were separated and organic layer evaporated. Residue was dissolved in EtOAc (30 mL), water (30 mL) was added, and pH of the resulting mixture adjusted to 4.0 (1N HCl). Layers were separated, and to the aqueous one fresh CH₂Cl₂ (30 mL) was added, and pH adjusted to 9.5 (aqueous NH₄OH). Layers were separated, to the organic layer water (10 mL) was added, and pH adjusted to 5.1 (1N HCl). Layers were separated and organic layer evaporated. Residue was purified by Biotage SP1 system (10 g cartridge, using 2% DEA/EtOAc-hexane) to afford title product (80 mg) as a single isomer.

HRMS (ES+) theoret. [M+H]⁺ C₃₉H₆₉N₂O₁₂ 773.4800, determined 773.4825 [M+H]⁺.

¹³C NMR (125 MHz, CDCl₃) [δ/ppm] 221.9, 175.4, 155.8, 99.9, 96.4, 84.4, 80.3, 79.9, 77.7, 76.9, 74.6, 74.4, 72.7, 70.2, 68.8, 65.7, 62.7, 51.1, 49.4, 44.7, 44.6, 45.1, 38.3, 38.0, 37.7, 36.3, 34.9, 32.0, 26.8, 21.4, 21.0, 20.8, 18.2, 18.2, 16.6, 16.2, 15.9, 12.0, 10.5, 8.5.

### Example 33

### N′-Benzyl-2′-O,3′-N-(carbonimidoyl)-3′-N-demethyl-erythromycin A

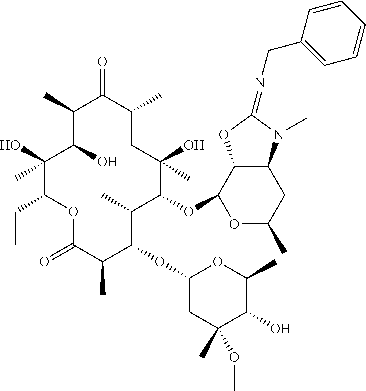

A mixture of Intermediate 8 (300 mg, 0.42 mmol), and benzyl isothiocyanate (166 μL, 1.25 mmol) in acetonitrile (15 mL) was stirred at 60° C. for 2 hours. Then EDC (240 mg, 1.25 mmol) was added and stirring continued at 60° C. overnight. Solvent was evaporated and the residue purified by Biotage SP1 system (10 g cartridge, using CH₂Cl₂-MeOH/CH₂Cl₂/NH₄OH (7.5:90:0.75)) to obtain crude product which was precipitated from EtOAc/hexane mixture to afford title product (25 mg) as a single isomer.

HRMS (ES+) theoret. [M+H]⁺ C₄₄H₇₁N₂O₁₃ 835.4956, determined 835.4968 [M+H]⁺.

¹³C NMR (125 MHz, CDCl₃) [δ/ppm] 221.7, 175.5, 156.3, 141.9, 127.9, 127.4, 126.0, 99.8, 96.4, 84.2, 80.3, 80.2, 77.6, 76.9, 74.6, 74.4, 72.7, 70.2, 68.8, 65.6, 62.6, 51.1, 50.0, 49.4, 44.6, 45.1, 38.4, 38,0, 37.7, 36.3, 34.9, 31.9, 26.7, 21.4, 21.0, 20.8, 18.2, 16.3, 15.9, 12.0, 10.5, 8.6.

### Example 34

### 2′-O,3′-N-(Carbonimidoyl)-3′-N-demethyl-N′-isopropyl-6-O-methyl-8a-aza-8a-homoerythromycin A

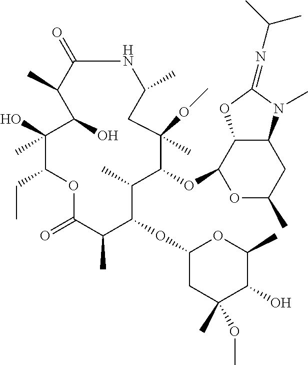

A mixture of Intermediate 9 (584 mg, 0.78 mmol) and isopropyl isothiocyanate (499 μL, 4.68 mmol) in acetonitrile (30 mL) was stirred at 60° C. for 3 hours. EDC (448 mg, 2.33 mmol) was added and stirring continued at 60° C. overnight. Solvent was evaporated, residue dissolved in CH₂Cl₂ (40 mL), water (40 mL) was added, and pH adjusted to 4.0 (1N HCl). Layers were separated, to the aqueous one CH₂Cl₂ (10 mL) was added, and pH of the resulting mixture adjusted to 6.5 (aqueous NH₄OH). Layers were separated, organic layer evaporated and the residue purified by Biotage SP1 system (10 g cartridge, using CH₂Cl₂-MeOH/CH₂Cl₂/NH₄OH (7.5:90:0.75)) to afford title product (66 mg) as a single isomer.

HRMS (ES+) theoret. [M+H]⁺ C₄₁H₇₄N₃O₁₃ 816.5222, determined 816.5250 [M+H]⁺.

¹³C NMR (125 MHz, CDCl₃) [δ/ppm] 176.8, 174.9, 154.5, 100.3, 95.6, 81.5, 79.6, 78.5, 77.9, 77.9, 77.0, 74.2, 72.9, 70.3, 70.3, 65.5, 62.7, 51.8, 49.3, 46.5, 45.4, 42.8, 42.4, 41.7, 41.1, 36.4, 34.8, 32.3, 24.6, 24.4 23.7, 21.5, 21.5, 21.2, 20.8, 17.9, 16.0, 15.3, 11.0, 9.6, 8.7.

### Example 35

### N′-Benzyl-2′-O,3′-N-(carbonimidoyl)-3′-N-demethyl-6-O-methyl-8a-aza-8a-homoerythromycin A

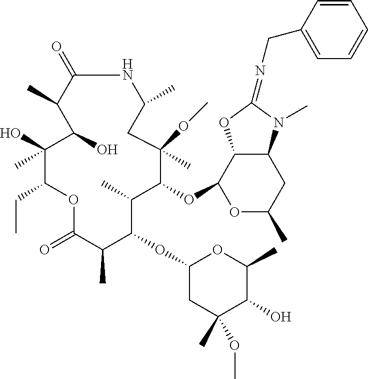

A mixture of Intermediate 9 (120 mg, 0.16 mmol) and benzyl isothiocyanate (64 μL, 0.48 mmol) in acetonitrile (8 mL) was stirred at 60° C. for 3 hours. Then, EDC (92 mg, 0.48 mmol) was added and stirring continued at 60° C. overnight. Solvent was evaporated, residue dissolved in CH₂Cl₂ (10 mL), water (10 mL) was added, and pH adjusted to 6.5 (aqueous NH₄OH). Layers were separated, organic layer evaporated and the residue purified by Biotage SP1 system (first: 10 g cartridge, using CH₂Cl₂-MeOH/CH₂Cl₂/NH₄OH (7.5:90:0.75), and then 10 g cartridge, using 2% DEA/EtOAc-hexane) and then precipitated from EtOAc/hexane to afford title product (30 mg) as a single isomer.

HRMS (ES+) theoret. [M+H]⁺ C₄₅H₇₄N₃O₁₃ 864.5222, determined 864.5223 [M+H]⁺.

¹³C NMR (125 MHz, CDCl₃) [δ/ppm] 177.3, 174.1, 156.4, 141.9, 127.9, 127.3, 126.0, 100,0, 95.3, 81.0, 80.0, 78.5, 77.9, 77.0, 77.0, 74.2, 72.9, 70.3, 70.2, 65.5, 62.7, 51.9, 50.0, 49.3, 45.4, 42.9, 42.4, 41.9, 40.9, 36.4, 34.6, 32.0, 23.8, 21.5, 21.4, 20.7, 20.6, 18.0, 16.1, 15.1, 11.1, 9.5, 8.7.

### Example 36

### N′-Benzyl-2′-O,3′-N-(carbonimidoyl)-3′-N-demethyl-9-deoxo-9a-aza-9a-homoerythromycin A

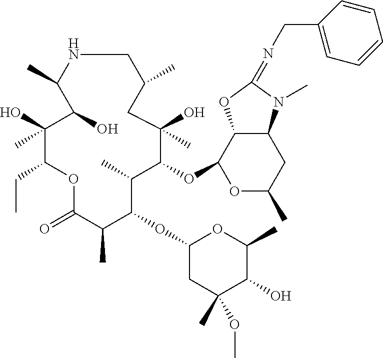

A mixture of Intermediate 10 (750 mg, 1.04 mmol) and benzyl isothiocyanate (110 μL, 0.83 mmol) in acetonitrile (20 mL) was stirred at 60° C. for 2 hours. Then EDC (199 mg, 1.04 mmol) was added and stirring continued at 60° C. overnight. Solvent was evaporated, residue dissolved in CH₂Cl₂ (40 mL), and washed with water (40 mL). To the organic layer water (40 mL) was added and pH of the resulting mixture adjusted to 4.0 (1N HCl). Layers were separated, to the aqueous layer CH₂Cl₂ (40 mL) was added, and pH of the resulting mixture adjusted to 6.5 (aqueous NH₄OH). Layers were separated, organic layer was evaporated, and the residue was purified by Biotage SP1 system (10 g cartridge, first using CH₂Cl₂-MeOH/CH₂Cl₂/NH₄OH (7.5:90:0.75), and then using 2% DEA/EtOAc-hexane) and then precipitated from EtOAc/hexane to afford title product (330 mg) as a single isomer.

HRMS (ES+) theoret. [M+H]⁺ C₄₄H₇₄N₃O₁₂ 836.5273, determined 836.5265 [M+H]⁺.

¹³C NMR (125 MHz, CDCl₃) [δ/ppm] 178.5, 156.5, 142.1, 127.9, 127.4, 125.9, 99.9, 95.4, 84.6, 80.2, 78.8, 77.9, 77.9, 73.7, 73.3, 73.3, 73.0, 70.0, 65.6, 62.9, 57.0, 56.7, 50.1, 49.3, 45.0, 41.7, 41.1, 36.4, 34.8, 32.0, 29.6, 27.1, 21.8, 21.5, 20.8, 20.8, 18.0, 15.9, 15.3, 13.9, 11.0, 8.7.

It will be appreciated by the skill in the art that the title compound may be also prepared from Intermediate 11 in a similar manner to that described in Example 5 hereinabove.

### Example 37

### 2′-O,3′-N-(Carbonimidoyl)-3′-N-demethyl-N′-isopropyl-9-deoxo-9a-aza-9a-homoerythromycin A

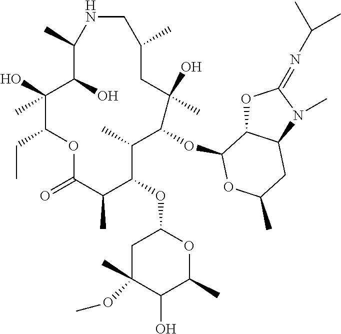

A mixture of Intermediate 10 (500 mg, 0.69 mmol) and isopropyl isothiocyanate (0.222 mL, 2.08 mmol) in acetonitrile (30 mL) was stirred at 40° C. for 24 hours. Then, EDC (399 mg, 2.08 mmol) was added and reaction mixture stirred at 60° C. overnight. Solvent was evaporated, residue dissolved in CH₂Cl₂ (50 mL), water (50 mL) was added, and pH or the resulting mixture adjusted to 6.5 (1N HCl). The aqueous layer was separated and further extracted with CH₂Cl₂ (3×50 mL). Combined organic layers were evaporated under reduced pressure to give crude product which was purified using Flashmaster II-solid phase extraction techniques (SPE 5 g) using gradient solvent system (100-90% hexane-EtOAc/DEA (9:1)) and 8 ml/min flow rate to afford product, which was further purified by mass directed autopreparative HPLC using acetonitrile-water (0.5% formic acid). Collected fractions were passed through a SAX column to remove formic acid and then freeze dried to afford title product (20 mg) as a single isomer; MS (ES+) m/z: 788.4 [M+H]⁺.

¹³C NMR (125 MHz, CDCl3) [d/ppm] 178.6, 154.8, 100.1, 95.3, 84.6, 79.7, 78.8, 77.9, 77.9, 73.2, 73.4, 73.0, 73.6, 70.1, 57.0, 65.7, 62.9, 56.8, 49.3, 46.5, 45.1, 41.6, 41.2, 36.4, 34.8, 32.3, 27.0, 29.6, 24.6, 24.4, 21.8, 21.5, 20.7, 20.8, 17.9, 15.3, 15.9, 13.7, 11.0, 8.5.

It will be appreciated by the skill in the art that the title compound may be also prepared from Intermediate 12 in a similar manner to that described in Example 5 hereinabove.

### Example 38

### N′-Benzyl-2′-O,3′-N-(carbonimidoyl)-3′-N-demethyl-6-O-methyl-9-deoxo-8a-methyl-8a-aza-8a-homoerythromycin A

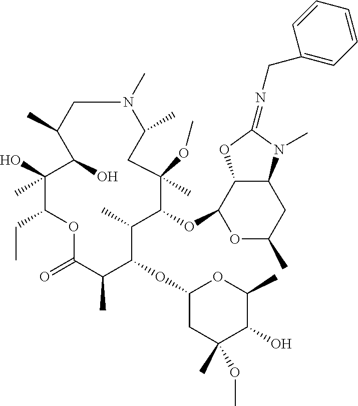

A mixture of Intermediate 13 (120 mg, 0.16 mmol) and benzyl isothiocyanate (21 μL, 0.16 mmol) in acetonitrile (10 mL) was stirred at 60° C. for 1 hour. Then EDC (31 mg, 0.16 mmol) was added and stirring continued at 60° C. overnight. Additional amount of benzyl isothiocyanate (42 μL, 0.32 mmol) was added and reaction mixture further stirred at 60° C. for 5 hours. Then, additional amount of EDC (62 mg, 0.32 mmol) was added and stirring continued overnight at 60° C. Then, solvent was evaporated, residue dissolved in CH₂Cl₂ (40 mL), water (40 mL) was added, and pH adjusted to 4.0 (1N HCl). Layers were separated, to aqueous one CH₂Cl₂ (40 mL) was added and pH adjusted to 5.2 (aqueous NH₄OH). Layers were separated and organic layer was again extracted with water (20 mL). To combined aqueous layers CH₂Cl₂ (40 mL) was added and pH adjusted to 7.0 (aqueous NH₄OH). Layers were separated and organic layer was evaporated. The obtained residue was purified by Biotage SP1 system (10 g cartridge, using CH₂Cl₂-MeOH/CH₂Cl₂/NH₄OH (7.5:90:0.75)) to afford title product (40 mg) as a single isomer.

HRMS (ES+) theoret. [M+H]⁺ C₄₆H₇₈N₃O₁₂ 864.5586, determined 864.5587 [M+H]⁺.

¹³C NMR (125 MHz, CDCl₃) [δ/ppm] 177.0, 156.4, 142.0, 127.9, 127.4, 126.0, 99.7, 95.7, 80.2, 80.2, 79.0, 78.1, 77.8, 77.2, 76.2, 72.9, 70.1, 65.7, 62.7, 55.6, 50.9, 50.1, 49.3, 45.4, 40.5, 40.5, 36.4, 34.8, 32.3, 32.1, 22.3, 20.8, 21.5, 20.8, 18.1, 16.3, 15.7, 14.3, 14.1, 11.4, 9.0.

### Example 39

### 2′-O,3′-N-(Carbonimidoyl)-3′-N-demethyl-N′-isopropyl-6-O-methyl-9-deoxo-8a-methyl-8a-aza-8a-homoerythromycin A

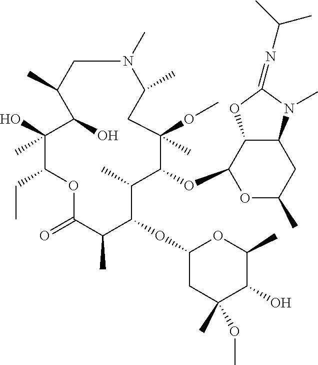

A mixture of Intermediate 13 (130 mg, 0.17 mmol) and isopropyl isothiocyanate (19 μL, 0.17 mmol) in acetonitrile (8 mL) was stirred at 60° C. for 1 hour. Then, EDC (67 mg, 0.35 mmol) was added and stirring continued at 60° C. overnight. Solvent was evaporated, residue dissolved in CH₂Cl₂ (40 mL), and water (40 mL) was added. Layers were separated, to the organic layer water (50 mL) was added and pH adjusted to 4.2 (1N HCl). Layers were separated and aqueous layer additionally extracted with CH₂Cl₂ (20 mL). Then to the aqueous layer CH₂Cl₂ (40 mL) was added and pH adjusted to 7.0 (aqueous NH₄OH). Layers were separated and organic layer evaporated. The residue was purified by Biotage SP1 system (10 g cartridge, using CH₂Cl₂-MeOH/CH₂Cl₂/NH₄OH (7.5:90:0.75)) to afford title product (30 mg) as a single isomer.

HRMS (ES+) theoret. [M+H]⁺ C₄₂H₇₈N₃O₁₂ 816.5586, determined 816.5607 [M+H]⁺.

¹³C NMR (125 MHz, CDCl₃) [δ/ppm] 177.0, 156.4, 99.9, 95.7, 80.3, 79.7, 79.0, 78.1, 77.8, 77.3, 76.1, 72.9, 70.1, 65.6, 62.8, 50.9, 49.3, 46.5, 45.4, 40.5, 40.2, 36.4, 34.8, 32.3, 24.7, 24.4, 22.3, 21.5, 20.8, 18.0, 16.2, 14.1, 11.4, 8.8.

### Example 40

### 2′-O,3′-N-(Carbonimidoyl)-3′-N-demethyl-N′-isopropyl-9-deoxo-9a-aza-9a-propyl-9a-homoerythromycin A

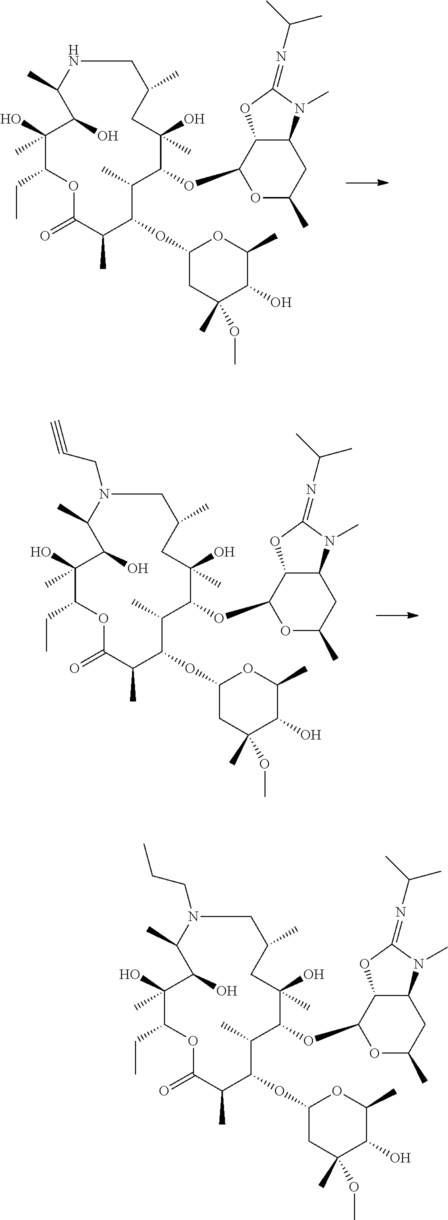

### Step a) 2′-O,3′-N-(Carbonimidoyl)-3′-N-demethyl-N′-isopropyl-9-deoxo-9a-aza-9a-(2-propyn-1-yl)-9a-homoerythromycin A

A solution of 2′-O,3′-N-(carbonimidoyl)-3′-N-demethyl-N′-isopropyl-9-deoxo-9a-aza-9a-homoerythromycin A, Example 37 (240 mg, 0.31 mmol), propargyl bromide (34 μL, 0.31 mmol), and DIPEA (160 μL, 0.91 mmol) in DMF (4 mL) was heated at 80° C. under microwave irradiation for 20 minutes. Additional amount of propargyl bromide (170 μL, 1.55 mmol) was added and the reaction mixture heated at 80° C. under microwave irradiation for 10 minutes. Then, additional amount of propargyl bromide (136 μL, 1.24 mmol) and DIPEA (107 μL, 1.55 mmol) were added and reaction mixture heated at 80° C. under microwave irradiation for 15 minutes. Then EtOAc (8 mL) was added, washed with water (2×10 mL), and dried over anhydrous Na₂SO₄. Solvent was evaporated and residue purified by Biotage SP1 system (10 g cartridge, using CH₂Cl₂-MeOH/CH₂Cl₂/NH₄OH (7.5:90:0.75)) to afford title product (170 mg) as a single isomer.

HRMS (ES+) theoret. [M+H]⁺ C₄₃H₇₆N₃O,₂ 826.5429, determined 826.5452 [M+H]⁺.

¹³C NMR (125 MHz, CDCl₃) [δ/ppm] 178.4, 154.7, 100.0, 95.1, 84.6, 80.0, 79.7, 78.5, 77.9, 77.6, 74.2, 74.2, 73.7, 73.0, 70.2, 65.7, 63.9, 62.9, 62.0, 49.3, 46.5, 44.9, 42.3, 41.2, 37.2, 36.3, 34.7, 32.3, 26.9, 26.2, 24.6, 22.0, 21.5, 21.2, 20.8, 17.9 16.2, 15.0, 11.1, 10.0, 8.5.

### Step b) 2′-O,3′-N-(Carbonimidoyl)-3′-N-demethyl-N′-isopropyl-9-deoxo-9a-aza-9a-propyl-9a-homoerythromycin A

A suspension of 2′-O,3′-N-(carbonimidoyl)-3′-N-demethyl-N′-isopropyl-9-deoxo-9a-aza-9a-(2-propyn-1-yl)-9a-homoerythromycin A from Step a (140 mg, 0.17 mmol) and 5% Pd/C (20 mg, 9.4 μmol) in ethanol (30 mL) was hydrogenated at RT for 40 minutes. Then, catalyst was removed by filtration, solvent evaporated, and residue purified by Biotage SP1 system (10 g cartridge, using CH₂Cl₂-MeOH/CH₂Cl₂/NH₄OH (7.5:90:0.75)) to afford title product (30 mg) as a single isomer.

HRMS (ES+) theoret. [M+H]⁺ C₄₃H₈₀N₃O₁₂ 830.5742, determined 830.5759 [M+H]⁺.

¹³C NMR (125 MHz, CDCl₃) [δ/ppm] 178.1, 154.7, 100.0, 95.7, 84.5, 79.8, 79.5, 77.8, 77.8, 74.6, 74.1, 73.8, 72.9, 70.0, 65.7, 64.9, 62.9, 61.6, 52.5, 49.3, 46.5 , 44.8, 40.7, 40.0, 36.4, 34.9, 32.3, 28.6, 27.1, 24.6, 22.5, 21.5, 21.2, 20.6, 20.6, 18.0, 16.2, 15.3, 12.1, 11.1, 8.7.

### Example 41

### 2′-O,3′-N-(Carbonimidoyl)-3′-N-demethyl-N′-methyl-9-deoxo-9a-methyl-9a-aza-9a-homoerythromycin A

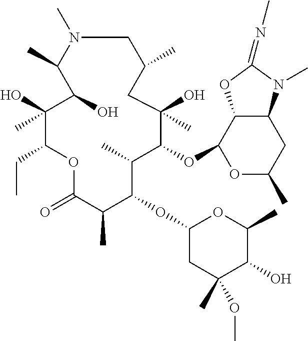

A mixture of Intermediate 3 (0.4 g, 0.54 mmol) and methyl isothiocyanate (60 mg, 0.82 mmol) in acetonitrile (20 mL) was stirred at 60° C. for 2 hours. Then, TEA (76 μl, 0.54 mmol) and CuCl₂ (73 mg, 0.54 mmol) were added and reaction mixture was additionally stirred at 60° C. for 2 hours. The solvent was evaporated, residue dissolved in CH₂Cl₂ (40 mL), water (40 mL) was added, and pH adjusted to 8.0 (aqueous NH₄OH). Layers were separated, organic layer was evaporated and residue purified by Biotage SP1 system (10 g cartridge, first using CH₂Cl₂-MeOH/CH₂Cl₂/NH₄OH (7.5:90:0.75)) and then 10 g cartridge, using 2% DEA/EtOAc-hexane) to afford title product (100 mg) as a single isomer.

HRMS (ES+) theoret. [M+H]⁺ C₃₉H₇₂N₃O₁₂ 774.5116, determined 774.5126 [M+H]⁺.

¹³C NMR (125 MHz, CDCl₃) [δ/ppm] 178.6, 156.8, 99.9, 95.2, 84.6, 80.0, 78.8, 77.9, 77.5, 74.6, 74.2, 73.2, 73.0, 70.0, 70.0 65.7, 62.9, 62.2, 49.3, 45.0, 41.9, 40.9, 36.3, 36.3, 34.8, 33.1, 31.9, 28.6, 27.2, 22.5, 21.5, 21.0, 20.7, 18.0, 16.2, 15.3, 11.1, 7.3, 8.4.

### Example 42

### 2′-O,3′-N-(Carbonimidoyl)-3′-N-demethyl-N′-ethyl-9-deoxo-9a-methyl-9a-aza-9a-homoerythromycin A

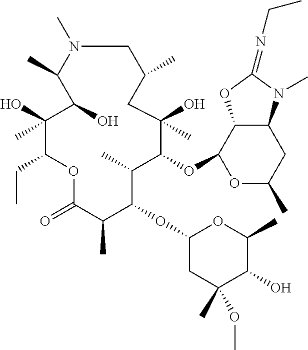

A mixture of Intermediate 3 (0.9 g, 1.23 mmol) and ethyl isothiocyanate (322 μL, 3.68 mmol) in acetonitrile (35 mL) was stirred at 60° C. for 2 hours. Then EDC (1.6 g, 8.57 mmol) was added and stirring at 60° C. continued overnight. Solvent was evaporated, residue dissolved in CH₂Cl₂ (50 mL), water (50 mL) was added, and pH adjusted to 5.2 (1N HCl). Layers were separated and aqueous layer was additionally extracted with CH₂Cl₂ (2×30 mL). Then to the aqueous layer CH₂Cl₂ (50 mL) was added and pH adjusted to 6.1 (aqueous NH₄OH). Layers were separated and aqueous layer was additionally extracted with CH₂Cl₂ (2×20 mL). The combined organic layers were dried over anhydrous Na₂SO₄. Evaporation of the solvent afforded crude product which was purified by Biotage SP1 system (10 g cartridge, using CH₂Cl₂-MeOH/CH₂Cl₂/NH₄OH (7.5:90:0.75)) and then by precipitation from mixture of acetone/water to afford title product (311 mg) as a single isomer.

HRMS (ES+) theoret. [M+H]⁺ C₄₀H₇₄N₃O₁₂ 788.5277, determined 788.5301 [M+H]⁺.

¹³C NMR (125 MHz, CDCl₃) [δ/ppm] 178.5, 155.9, 99.9, 95.6, 84.8, 80.0, 79.0, 77.9, 77.6, 74.2, 74.5, 73.3, 73.2, 70.1, 70.1 65.7, 62.8, 62.1, 49.3, 44.9, 41.9, 40.7, 40.6, 36.4, 36.4, 35.3, 32.1, 28.6, 26.6, 21.9, 21.5, 21.0, 20.8, 18.0, 16.6, 16.1, 15.3, 11.1, 7.3, 8.5.

### Example 43

### 2′-O,3′-N-(Carbonimidoyl)-3′-N-demethyl-N′-[3-(methylthio)propyl]-9a-methyl-9a-aza-9a-homoerythromycin A

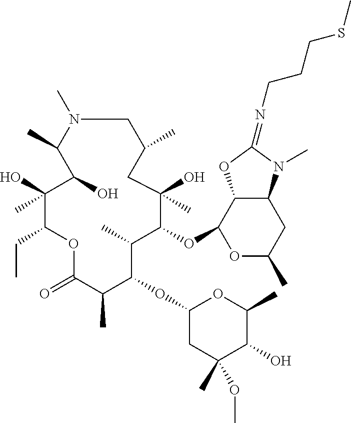

A mixture of Intermediate 3 (1 g, 1.361 mmol) and 3-(methylthio)propyl isothiocyanate (0.545 mL, 4.08 mmol) in acetonitrile (50 mL) was stirred at 60° C. for 1 hour. Then EDC (0.783 g, 4.08 mmol) was added and stirring continued at 60° C. overnight. Additional amount of EDC (0.783 g, 4.08 mmol) was added and reaction mixture stirred at 60° C. overnight. Precipitate was filtered off, and filtrate evaporated. The residue was dissolved in CH₂Cl₂ (50 mL), water was added (50 mL), and pH adjusted to 6.5 (1N HCl). Layers were separated, to the organic layer water (50 mL) was added and pH adjusted to 4.1. Layers were separated and aqueous layer additionally extracted with CH₂Cl₂ (50 mL). To the aqueous layer CH₂Cl₂ (50 mL) was added, and pH adjusted to 9.2 (aqueous NH₄OH). Layers were separated and aqueous layer additionally extracted with CH₂Cl₂ (3×50 mL). Combined organic layers at pH 9.2 were dried over anhydrous K₂CO₃, solvent was evaporated to afford crude product which was precipitated from diethylether and purified by silica gel column chromatography (using CH₂Cl₂/MeOH/NH₄OH (90:5:0.5)) to afford title product (214 mg) as a single isomer.

HRMS (ES+) theoret. [M+H]⁺ C₄₂H₇₈N₃O₁₂S 848.5306, determined 848.5292 [M+H]⁺.

¹³C NMR (125 MHz, CDCl₃) [δ/ppm] 178.7, 159.1, 99.9, 94.9, 84.6, 79.9, 78.5, 77.9, 77.5, 74.1, 73.9, 73.3, 73.0, 70.1, 70.0, 65.7, 62.9, 62.3, 49.4, 45.4, 45.1, 41.9, 41.3, 36.3, 36.2, 34.7, 32.1, 32.1, 31.1, 27.3, 26.7, 21.9, 21.5, 21.1, 20.7, 17.9, 16.1, 15.4, 14.9, 11.1, 8.4, 7.1.

### Example 44

### 2′-O,3′-N-(Carbonimidoyl)-3′-N-demethyl-N′-[4-(metoxy)phenyl]-9a-methyl-9a-aza-9a-homoerythromycin A

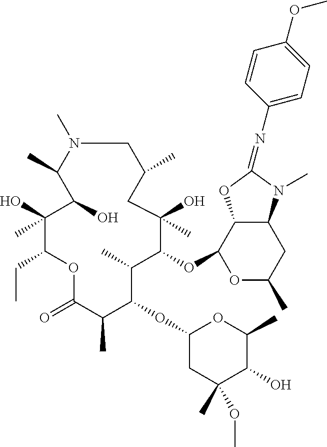

A mixture of Intermediate 3 (1 g, 1.361 mmol) and 4-metoxyphenyl isothiocyanate (0.570 mL, 4.08 mmol) in acetonitrile (50 mL) was stirred at 60° C. for 1 hour. Then, EDC (0.783 g, 4.08 mmol) was added and stirring at 60° C. continued overnight. Additional amount of EDC (0.783 g, 4.08 mmol) was added and the reaction mixture stirred at 60° C. overnight. Precipitate was filtered off, and filtrate evaporated. The residue was dissolved in CH₂Cl₂ (50 mL), water was added (50 mL), and pH adjusted to 6.5 (1N HCl). Layers were separated, to the organic layer water (50 mL) was added and pH adjusted to 4.1. Layers were separated and organic layer evaporated. The residue was dissolved in EtOAc (50 mL) and extracted with water (50 mL). To the aqueous layer CH₂Cl₂ (50 mL) was added, and pH adjusted to 9.2 (aqueous NH₄OH), Layers were separated and aqueous layer additionally extracted with CH₂Cl₂ (3×50 mL). Combined organic layers at pH 9.2 were dried over K₂CO₃, solvent was evaporated to afford crude product which was purified by silica gel column chromatography (using CH₂Cl₂/MeOH/NH₄OH (90:5:0.5)) to afford title product (363 mg) as a single isomer.

HRMS (ES+) theoret. [M+H]⁺ C₄₅H₇₆N₃O₁₃ 866.5378, determined 866.5402 [M+H]⁺.

¹³C NMR (125 MHz, CDCl₃) [δ/ppm] 178.7, 155.0, 154.3, 139.6, 124.6, 113.5, 99.7, 95.0, 84.6, 80.4, 78.5, 77.9, 77.5, 74.1, 73.9, 73.2, 73.1, 70.0, 70.0, 65.7, 62.4, 62.3, 55.3, 49.5, 45.0, 41.9, 41.2, 36.2, 36.4, 34.7, 31.7, 27.2, 26.6, 21.8, 21.5, 21.1, 20.7, 18.0, 16.1, 15.0, 11.1, 8.5, 7.2.

### Example 45

### 2′-O,3′-N-(Carbonimidoyl)-3′-N-demethyl-N′-(tert-butyl)-9a-methyl-9a-aza-9a-homoerythromycin A

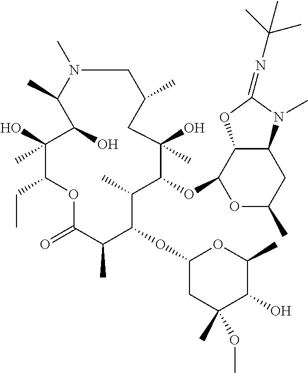

A mixture of Intermediate 3 (1 g, 1.361 mmol) and tert-butyl isothiocyanate (1.036 mL, 8.16 mmol) in acetonitrile (50 mL) was stirred at 60° C. overnight. Additional amount of tert-butyl isothiocyanate (0.173 mL, 1.361 mmol) was added stirreding at 60° C. continued for 8 hours. Then EDC (0.783 g, 4.08 mmol) was added and reaction mixture stirred at 60° C. overnight. Solvent was evaporated, the residue dissolved in CH₂Cl₂ (50 mL), water was added (50 mL), and pH adjusted to 6.5 (1N HCl). Layers were separated and organic layer evaporated. The residue was dissolved in EtOAc (50 mL), water was added, and pH adjusted to 4.1 (1N HCl). Layers were separated, to the aqueous layer CH₂Cl₂ (50 mL) was added, and pH adjusted to 9.2 (aq. NH₄OH). Layers were separated and aqueous layer additionally extracted with CH₂Cl₂ (2×50 mL). Combined organic layers at pH 9.2 were dried over K₂CO₃, solvent was evaporated to afford crude product which was purified by solid-phase extraction technique (SPE 20 g, using CH₂Cl₂/MeOH/NH₄OH (90:5:0.5)) to afford title product (143 mg) as a single isomer.

HRMS (ES+) theoret. [M+H]⁺ C₄₂H₇₈N₃O₁₂ 816.5586, determined 816.5581 [M+H]⁺.

¹³C NMR (125 MHz, CDCl₃) [δ/ppm] 178.7, 152.8, 100.0, 95.0, 84.6, 79.8, 78.6, 77.9, 77.5, 74.1, 73.9, 73.3, 73.0, 70.1, 70.0, 65.6, 62.3, 62.3, 51.7, 49.3, 45.1, 41.8, 41.3, 36.2, 36.4, 34.7, 32.7, 30.5, 27.2, 26.6, 21.8, 21.5, 21.0, 20.8, 17.9, 16.1, 15.1, 11.1, 8.9, 7.2.

### Example 46

### 2′-O,3′-N-(Carbonimidoyl)-3′-N-demethyl-9a-methyl-N′-(4-quinolinyl)-9a-aza-9a-homoerythromycin A

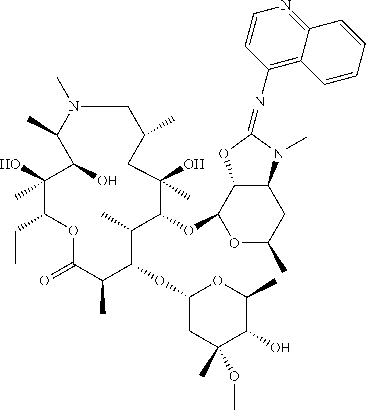

A mixture of Intermediate 3 (1 g, 1.361 mmol) and 4-quinolinyl isothiocyanate (0.760 g, 4.08 mmol) in acetonitrile (50 mL) was stirred at 60° C. for 2 hours. Then EDC (0.783 g, 4.08 mmol) was added and stirring at 60° C. continued overnight. Solvent was evaporated, residue dissolved in CH₂Cl₂ (50 mL), water was added (50 mL), and pH adjusted to 6.5 (aqueous NH₄OH). Layers were separated and organic layer evaporated. The residue was purified by Biotage SP1 system (10 g cartridge, using CH₂Cl₂-MeOH/CH₂Cl₂/NH₄OH (7.5:90:0.75)) to afford title product (116 mg) as a single isomer.

HRMS (ES+) theoret. [M+H]⁺ C₄₇H₇₅N₄O₁₂ 887.5381, determined 887.5362 [M+H]⁺.

¹³C NMR (125 MHz, CDCl₃) [δ/ppm] 178.6, 156.1, 151.2, 150.4, 149.2, 128.9, 128.8, 125.7, 124.9, 124.6, 124.1, 112.9, 99.4, 95.2, 84.9, 81.1, 78.8, 77.8, 77.5, 74.2, 74.1, 73.1, 73.2, 70.1, 70.0, 65.7, 62.2, 62.1, 49.5, 44.9, 41.8, 40.8, 36.3, 36.3, 34.8, 31.3, 27.1, 26.6, 21.8, 21.5, 20.9, 20.7 18.0, 16.0, 15.2, 11.1, 8.4, 7.2.

### Example 47

### 2′-O,3′-N-(Carbonimidoyl)-3′-N-demethyl-9a-methyl-N′-[2-(4-morpholinyl)ethyl]-9a-aza-9a-homoerythromycin A

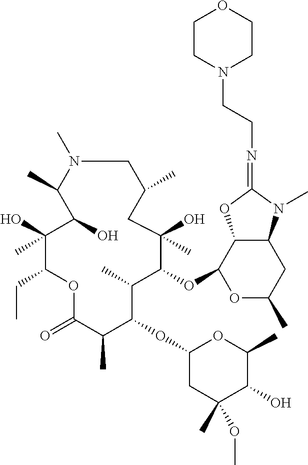

A mixture of Intermediate 3 (1 g, 1.361 mmol) and 2-(4-morpholino)ethyl isothiocyanate (0.703 g, 4.08 mmol) in acetonitrile (50 mL) was stirred at 60° C. for 1 hour. Then EDC (0.783 g, 4.08 mmol) was added and stirring at 60° C. continued overnight. Solvent was evaporated, residue dissolved in CH₂Cl₂ (50 mL), water was added (50 mL), and pH adjusted to 6.5 (1N HCl). Layers were separated, to the organic layer water (50 mL) was added, and pH adjusted to 9.2 (aqueous NH₄OH). Layers were separated and aqueous layer additionally extracted with CH₂Cl₂ (3×50 mL). Combined organic layers were dried over K₂CO₃, solvent was evaporated to afford crude product which was purified by silica gel column chromatography (CH₂Cl₂/MeOH/NH₄OH (90:5:0.5)) to afford title product (63 mg) as a single isomer; MS (ES+) m/z: 873.88 [M+H]⁺.

¹³C NMR (125 MHz, CDCl₃) [δ/ppm] 178.6, 156.4, 99.8, 94.9, 84.4, 80.1, 78.4, 77.9, 77.5, 74.1, 73.9, 73.2, 73.1, 70.0, 70.0, 66.9, 65.7, 62.8, 62.3, 60.1, 53.9, 49.4, 45.0, 43.5, 41.9, 41.3, 36.4, 36.2, 34.7, 31.9, 27.3, 26.7, 21.9, 21,5, 21.1, 20.7, 17.9, 16.1, 14.9, 11.1, 8.5, 7.2.

### Example 48

### N′-Benzyl-2′-O,3′-N-(carbonimidoyl)-3′-N-demethyl-9-deoxo-9a-aza-9a-propyl-9a-homoerythromycin A

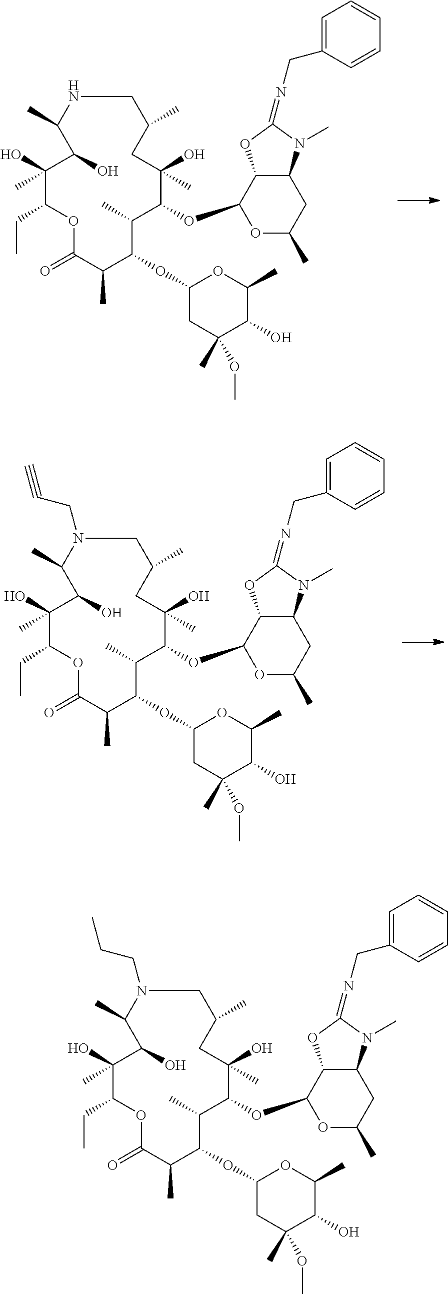

### Step a) N′-Benzyl-2′-O,3′-N-(carbonimidoyl)-3′-N-demethyl-9-deoxo-9a-aza-9a-(2-propyn-1-yl)-9a-homoerythromycin A

A solution of N′-benzyl-2′-O,3′-N-(carbonimidoyl)-3′-N-demethyl-9-deoxo-9a-aza-9a-homoerythromycin A, Example 36 (260 mg, 0.31 mmol), propargyl bromide (346 μL, 3.11 mmol), and DIPEA (272 μL, 1.56 mmol) in DMF (4 mL) was heated at 80° C. under microwave irradiation for 20 minutes. Then EtOAc (8 mL) was added, washed with water (10 mL), and dried over anhydrous Na₂SO₄. Solvent was evaporated and residue purified by Biotage SP1 system (10 g cartridge, using CH₂Cl₂-MeOH/CH₂Cl₂/NH₄OH (7.5:90:0.75)) to afford title product (64 mg) as a single isomer.

HRMS (ES+) theoret. [M+H]⁺ C₄₇H₇₆N₃O₁₂ 874.5429, determined 874.5446 [M+H]⁺.

¹³C NMR (125 MHz, CDCl₃) [δ/ppm] 178.4, 156.5, 142.0, 127.9, 127.4, 126.0, 99.8, 95.2, 84.5, 80.1, 80.1, 78.5, 77.9, 77.6, 74.4, 74.3, 74.3, 73.7, 73.0, 70.1, 65.7, 63.9, 62.8, 62.0, 50.1, 49.3, 44.9, 42.3, 41.2, 37.2, 36.4, 34.7, 32.0, 26.8, 26.2, 22.0, 21.5, 21.3, 20.7, 18.0, 16.2, 14.9, 11.2, 10.1, 8.7.

### Step b) N′-Benzyl-2′-O,3′-N-(carbonimidoyl)-3′-N-demethyl-9-deoxo-9a-aza-9a-propyl-9a-homoerythromycin A

A suspension of N′-benzyl-2′-O,3′-N-(carbonimidoyl)-3′-N-demethyl-9-deoxo-9a-aza-9a-(2-propyn-1-yl)-9a-homoerythromycin A from Step a (260 mg, 0.30 mmol) and 5% Pd/C (26 mg, 12.0 μmol) in ethanol (30 mL) was hydrogenated at RT for 2 hours. Then, catalyst was removed by filtration, solvent evaporated, and residue purified by Biotage SP1 system (10 g cartridge, using CH₂Cl₂-MeOH/CH₂Cl₂/NH₄OH (7.5:90:0.75)) to afford title product (25 mg) as a single isomer.

MS (ES+) m/z: 878.75 [M+H]⁺.

¹³C NMR (125 MHz, CDCl₃) [δ/ppm] 178.0, 156.5, 142.0, 127.9, 127.4, 126.0, 99.8, 95.7, 84.6, 80.2, 79.5, 77.8, 74.6, 74.1, 73.9, 72.9, 70.0, 65.7, 65.0, 62.8, 61.6, 52.4, 50.0, 49.3, 44.8, 40.9, 40.0, 36.4, 34.9, 31.8, 28.5, 27.1, 22.5, 21.5, 21.2, 20.8, 20.6, 18.0, 16.2, 15.2, 12.0, 11.1, 9.1, 8.8.

In Vitro Assay

The in vitro potency of the compounds has been measured using the methodology described in the in vitro protocol for Inhibition of IL-6 production in LPS-stimulated murine splenocytes in vitro. Compounds of examples 4, 8, 15 and 24 exhibited more than 40% of inhibition of interleukin-6 (IL-6) production, compounds of examples 7, 14, 22, 23, 34 and 47 exhibited more than 60% of inhibition of interleukin-6 (IL-6) production and compounds of examples 1 to 3, 5, 6, 9 to 13, 16, 17, 18, 20, 26 to 33, 35 to 46 and 48 exhibited more than 80% of inhibition of interleukin-6 (IL-6) production in LPS-stimulated splenocytes at 50 μM or/and 25 μM concentration of the compound.

In Vivo Assay

The in vivo potency of the compounds has been measured using the methodology described in the in vivo protocol for Lung neutrophilia induced by bacterial lipopolysaccharide in male BALB/cJ mice and/or using the methodology described in the in vivo protocol Phorbol 12-myristate 13-acetate induced ear edema in CD1 mice. Compounds of examples 3, 7, 11, 14 and 24 showed more than 70% inhibition and compounds of examples 5, 6 and 9 showed more than 50% inhibition of total cell number and number of neutrophils in BALF of treated animals which received intraperitoneally (i.p.) a single dose of 200 mg/kg of test compound. Compounds of examples 1 to 3, 5 to 8, and 11 to 15 showed more than 50% inhibition of edema applied topically once in a dose 500 μg/ear.

What is claimed is:

1. A compound of Formula (I): 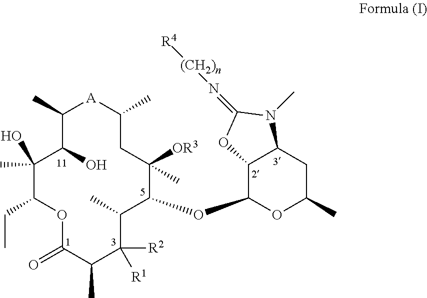
A is a bivalent radical selected from —C(O)—, —N(R⁵)CH₂—, —CH₂N(R⁵)—, —NHC(O)—, —C(O)NH—, —CH(OH)— and —C(═NOR⁶)—;
R¹ is the α-L-cladinosyl group of formula (II); 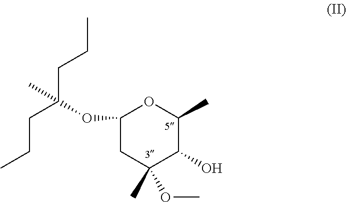
R² is hydrogen;
R³ is hydrogen or C₁₋₃alkyl;
R⁴ is
C₁₋₄ alkyl optionally substituted by hydroxyl, methoxy or thiomethyl;
(ii) N,N-di(C₁-C₃-alkyl)amino;
(iii) C₆₋₁₀aryl optionally substituted by one or two groups selected from C₁₋₃alkyl, halogen, hydroxyl, C₁₋₃alkyloxy and CF₃;
(iv) a 3-6 membered monocyclic heterocyclic ring or a fused 9-10 membered bicyclic heterocyclic ring which is saturated or partially unsaturated containing one or two heteroatoms selected from oxygen, nitrogen and sulphur;
(v) a 5-6 membered monocyclic heteroaromatic ring or a fused 9-10 membered bicyclic heteroaromatic ring containing 1 to 2 heteroatoms selected from oxygen, nitrogen and sulphur;
R⁵ is C₁₋₃alkyl or hydrogen;
R⁶ is hydrogen;
n is an integer from zero to 3 provided that ‘n’ cannot be zero when R⁴ is N,N-di(C₁-C₃-alkyl)amino, or a heterocyclic ring or a heteroaromatic ring attached via an heteroatom;
or a salt thereof.

2. A compound as claimed in claim 1, wherein A is a bivalent radical selected from —N(R⁵)CH₂—, —C(O)— and —NHC(O)—.

3. A compound as claimed in claim 1, wherein A is a bivalent radical —N(R⁵)CH₂—, R⁵ is C₁₋₃alkyl and R³ is hydrogen.

4. A compound as claimed in claim 1, wherein A is a bivalent radical —C(O)— and R³ is hydrogen or C₁₋₃alkyl.

5. A compound as claimed in claim 1, wherein A is a bivalent radical —NHC(O)— and R³ is hydrogen or C₁₋₃alkyl.

6. A compound as claimed in claim 1, wherein R³ is hydrogen.

7. A compound as claimed in claim 1 wherein R³ is methyl.

8. A compound as claimed in claim 1 wherein R⁴ is C₁₋₄alkyl and n is zero.

9. A compound as claimed in claim 1 wherein R⁴ is C₆₋₁₀aryl.

10. A compound of Formula (I) as claimed in claim 1, selected from:
N′-benzyl-2′-O,3′-N-(carbonimidoyl)-3′-N-demethyl-9-deoxo-9a-methyl-9a-aza-9a-homoerythromycin A;
2′-O,3′-N-(carbonimidoyl)-3′-N-demethyl-9-deoxo-9a-methyl-N′-(1-naphthyl)-9a-aza-9a-homoerythromycin A;
2′-O,3′-N-(carbonimidoyl)-3′-N-demethyl-9-deoxo-N′-isopropyl-9a-methyl-9a-aza-9a-homoerythromycin A;
2′-O,3′-N-(carbonimidoyl)-3′-N-demethyl-9-deoxo-N′-[3-(diethylamino)propyl]-9a-methyl-9a-aza-9a-homoerythromycin A;
N′-(benzyl)-2′-O,3′-N-(carbonimidoyl)-3′-N-demethyl-6-O-methyl-9a-aza-9a-homoerythromycin A;
2′-O,3′-N-(carbonimidoyl)-3′-N-demethyl-6-O-methyl-N′-(1-naphthyl)-9a-aza-9a-homoerythromycin A;
2′-O,3′-N-(carbonimidoyl)-3′-N-demethyl-N′-isopropyl-6-O-methyl-9a-aza-9a-homoerythromycin A;
2′-O,3′-N-(carbonimidoyl)-3′-N-demethyl-N′-[3-(diethylamino)propyl]-6-O-methyl-9a-aza-9a-homoerythromycin A;
N′-benzyl-2′-O,3′-N-(carbonimidoyl)-3′-N-demethyl-6-O-methyl-erythromycin A;
2′-O,3′-N-(carbonimidoyl)-3′-N-demethyl-6-O-methyl-N′-(1-naphthyl)-erythromycin A;
2′-O,3′-N-(carbonimidoyl)-3′-N-demethyl-N′-isopropyl-6-O-methyl-erythromycin A;
2′-O,3′-N-(carbonimidoyl)-3′-N-demethyl-N′-[3-(diethylamino)propyl]-6-O-methyl-erythromycin A;
2′-O,3′-N-(carbonimidoyl)-3′-N-demethyl-N′-methyl-6-O-methyl-erythromycin A;
2′-O,3′-N-(carbonimidoyl)-3′-N-demethyl-N′-ethyl-6-O-methyl-erythromycin A;
2′-O,3′-N-(carbonimidoyl)-3′-N-demethyl-N′-ethyl-6-O-methyl-9a-aza-9a-homoerythromycin A;
2′-O,3′-N-(carbonimidoyl)-3′-N-demethyl-6-O-methyl-N′-(4-quinolyl)-9a-aza-9a-homoerythromycin A;
2′-O,3′-N-(carbonimidoyl)-3′-N-demethyl-N′-(2,6-difluorophenyl)-6-O-methyl-9a-aza-9a-homoerythromycin A;
2′-O,3′-N-(carbonimidoyl)-3′-N-demethyl-6-O-methyl-N′-(tert-butyl)-9a-aza-9a-homoerythromycin A;
2′-O,3′-N-(carbonimidoyl)-3′-N-demethyl-N′-methyl-6-O-methyl-9a-aza-9a-homoerythromycin A;
2′-O,3′-N-(carbonimidoyl)-3′-N-demethyl-6-O-methyl-N′-[2-(4-morpholinyl)ethyl]-9a-aza-9a-homoerythromycin A;
2′-O,3′-N-(carbonimidoyl)-3′-N-demethyl-N′-[3-(methyloxy)propyl]-6-O-methyl-9a-aza-9a-homoerythromycin A;
2′-O,3′-N-(carbonimidoyl)-3′-N-demethyl-N′-[3-(methylthio)propyl]-6-O-methyl-9a-aza-9a-homoerythromycin A;
2′-O,3′-N-(carbonimidoyl)-3′-N-demethyl-6-O-methyl-N′-[4-(methyloxy)phenyl]-9a-aza-9a-homoerythromycin A;
2′-O,3′-N-(carbonimidoyl)-3′-N-demethyl-6-O-methyl-N′-(tetrahydro-2-furanylmethyl)-9a-aza-9a-homoerythromycin A;
2′-O,3′-N-(carbonimidoyl)-3′-N-demethyl-N′-(2-furanylmethyl)-6-O-methyl-9a-aza-9a-homoerythromycin A;
2′-O,3′-N-(carbonimidoyl)-3′-N-demethyl-N′-isopropyl-(9S)-9-dihydroerythromycin A;
N′-benzyl-2′-O,3′-N-(carbonimidoyl)-3′-N-demethyl-(9S)-9-dihydroerythromycin A;
2′-O,3′-N-(carbonimidoyl)-3′-N-demethyl-N′-ethyl-6-O-methyl-(9S)-9-dihydroerythromycin A;
2′-O,3′-N-(carbonimidoyl)-3′-N-demethyl-N′-isopropyl-6-O-methyl-(9S)-9-dihydroerythromycin A;
N′-benzyl-2′-O,3′-N-(carbonimidoyl)-3′-N-demethyl-6-O-methyl-(9S)-9-dihydroerythromycin A;
2′-O,3′-N-(carbonimidoyl)-3′-N-demethyl-N′-isopropyl-erythromycin A;
2′-O,3′-N-(carbonimidoyl)-3′-N-demethyl-N′-ethyl-erythromycin A;
N′-Benzyl-2′-O,3′-N-(carbonimidoyl)-3′-N-demethyl-erythromycin A;
2′-O,3′-N-(carbonimidoyl)-3′-N-demethyl-N′-isopropyl-6-O-methyl-8a-aza-8a-homoerythromycin A;
N′-benzyl-2′-O,3′-N-(carbonimidoyl)-3′-N-demethyl-6-O-methyl-8a-aza-8a-homoerythromycin A;
N′-benzyl-2′-O,3′-N-(carbonimidoyl)-3′-N-demethyl-9-deoxo-9a-aza-9a-homoerythromycin A;
2′-O,3′-N-(carbonimidoyl)-3′-N-demethyl-N′-isopropyl-9-deoxo-9a-aza-9a-homoerythromycin A;
N′-benzyl-2′-O,3′-N-(carbonimidoyl)-3′-N-demethyl-6-O-methyl-9-deoxo-8a-methyl-8a-aza-8a-homoerythromycin A;
2′-O,3′-N-(carbonimidoyl)-3′-N-demethyl-N′-isopropyl-6-O-methyl-9-deoxo-8a-methyl-8a-aza-8a-homoerythromycin A;
2′-O,3′-N-(carbonimidoyl)-3′-N-demethyl-N′-isopropyl-9-deoxo-9a-aza-9a-propyl-9a-homoerythromycin A;
2′-O,3′-N-(carbonimidoyl)-3′-N-demethyl-N′-methyl-9-deoxo-9a-methyl-9a-aza-9a-homoerythromycin A;
2′-O,3′-N-(carbonimidoyl)-3′-N-demethyl-N′-ethyl-9-deoxo-9a-methyl-9a-aza-9a-homoerythromycin A;
2′-O,3′-N-(carbonimidoyl)-3′-N-demethyl-N′-[3-(methylthio)propyl]-9a-methyl-9a-aza-9a-homoerythromycin A;
2′-O,3′-N-(carbonimidoyl)-3′-N-demethyl-N′-[4-(metoxy)phenyl]-9a-methyl-9a-aza-9a-homoerythromycin A;
2′-O,3′-N-(carbonimidoyl)-3′-N-demethyl-N′-(tert-butyl)-9a-methyl-9a-aza-9a-homoerythromycin A;
2′-O,3′-N-(carbonimidoyl)-3′-N-demethyl-9a-methyl-N′-(4-quinolinyl)-9a-aza-9a-homoerythromycin A;
2′-O,3′-N-(carbonimidoyl)-3′-N-demethyl-9a-methyl-N′-[2-(4-morpholinyl)ethyl]-9a-aza-9a-homoerythromycin A; and
N′-benzyl-2′-O,3′-N-(carbonimidoyl)-3′-N-demethyl-9-deoxo-9a-aza-9a-propyl-9a-homoerythromycin A;
or a salt thereof.

11. A compound of Formula (I) or a salt thereof as claimed in claim 1 wherein the salt is a pharmaceutically acceptable salt.

12. A compound of Formula (III) 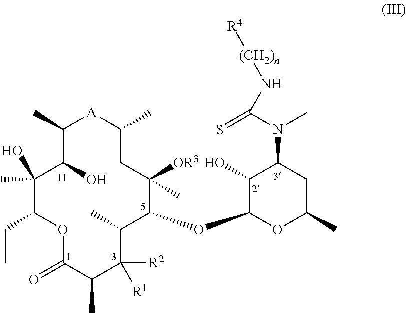
wherein,
A is a bivalent radical selected from —C(O)—, —N(R⁵)CH₂—, —CH₂N(R⁵)—, —NHC(O)—, —C(O)NH—, —CH(OH)— and —C(═NOR⁶)—;
R¹ is the α-L-cladinosyl group of formula (II); 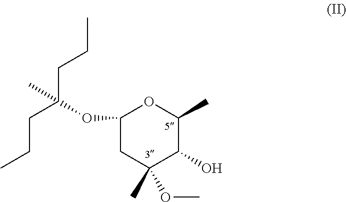
R² is hydrogen;
R³ is hydrogen or C₁₋₃alkyl;
R⁴ is
(i) C₁₋₄ alkyl optionally substituted by hydroxyl, methoxy or thiomethyl;
(ii) N,N-di(C₁-C₃-alkyl)amino;
(iii) C₆₋₁₀aryl optionally substituted by one or two groups selected from C₁₋₃alkyl, halogen, hydroxyl, C₁₋₃alkyloxy and CF₃;
(iv) a 3-6 membered monocyclic heterocyclic ring or a fused 9-10 membered bicyclic heterocyclic ring which is saturated or partially unsaturated containing one or two heteroatoms selected from oxygen, nitrogen and sulphur;
(v) a 5-6 membered monocyclic heteroaromatic ring or a fused 9-10 membered bicyclic heteroaromatic ring containing 1 to 2 heteroatoms selected from oxygen, nitrogen and sulphur;
R⁵ is C₁₋₃alkyl or hydrogen;
R⁶ is hydrogen;
n is an integer from zero to 3 provided that ‘n’ cannot be zero when R⁴ is N,N-di(C₁-C₃-alkyl)amino, or a heterocyclic ring or a heteroaromatic ring attached via an heteroatom;
or a salt thereof.

13. Process for the preparation of compounds of Formula (I) according to claim 1 comprising reacting a compound of Formula (III) 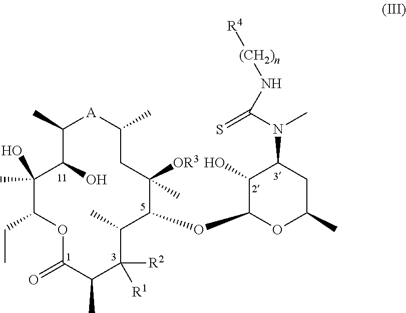
with an activating agent selected from carbodiimide and 2-chloro-1-methylpyridinium iodide.

14. A method for the treatment of neutrophil dominated inflammatory diseases resulting from neutrophilic infiltration and/or diseases associated with altered cellular functionality of neutrophils selected from chronic obstructive pulmonary disease, cystic fibrosis, diffuse panbronchiolitis, bronchiolitis obliterans, bronchitis, bronchiectasis, adult respiratory distress syndrome, severe or steroid-resistant asthma, emphysema, chronic rhinosinusitis, rheumatoid arthritis, gouty arthritis, inflammatory bowel disease, glomerulonephritis, damage from ischemic reperfusion, atherosclerosis, psoriasis, vasculitis, systemic lupus erythematosus, systemic inflammatory response syndrome, sepsis, ischemia-reperfusion injury, rosacea, periodontitis, gingival hyperplasia and prostatitis syndrome in a subject in need of such treatment comprising administering to the subject a therapeutically effective amount of compound of Formula (I) according to claim 1 or a pharmaceutically acceptable salt thereof.

15. The method of claim 14, wherein disease is selected from chronic obstructive pulmonary disease, cystic fibrosis, diffuse panbronchiolitis, bronchiolitis obliterans, bronchitis, bronchiectasis, adult respiratory distress syndrome, severe or steroid-resistant asthma, emphysema and chronic rhinosinusitis.

16. A pharmaceutical composition comprising a compound as claimed in claim 1 or a pharmaceutically acceptable salt thereof, in association with at least one pharmaceutically acceptable excipient, diluent and/or carrier.

17. (canceled)

18. (canceled)

19. (canceled)
